(19) United States
(12) Patent Application Publication
(10) Pub. No.: US 2026/0345604 A1
(43) Pub. Date: Nov. 5, 2026

(54) METHODS OF REDUCING NEURODEGENERATION ASSOCIATED WITH NEURODEGENERATIVE DISEASES

(71) Applicants: EISAI R&D MANAGEMENT CO., LTD., Tokyo (JP); WASHINGTON UNIVERSITY, St. Louis, MO (US)

(72) Inventors: Margaret L. MOLINE, Nutley, NJ (US); Jocelyn Y. CHENG, Nutley, NJ (US); Garth E. RINGHEIM, Nutley, NJ (US); Brendan P. LUCEY, St. Louis, MO (US); Erik S. MUSIEK, St. Louis, MO (US); David M. HOLTZMAN, St. Louis, MO (US); Carsten T. BEUCKMANN, Ibaraki (JP); Samira PARHIZKAR, St. Louis, MO (US); Ashish SHARMA, St. Louis, MO (US)

(21) Appl. No.: 19/113,535

(22) PCT Filed: Sep. 22, 2023
(86) PCT No.: PCT/US2023/074913

§ 371 Date: Mar. 20, 2025

Related U.S. Application Data
(60) Provisional application No. 63/376,949, filed on Sep. 23, 2022.
(60) Provisional application No. 63/382,278, filed on Nov. 3, 2022.

(51) Int. Cl. A61K 31/506; A61P 25/28; G01N 33/68

(52) U.S. Cl. CPC A61K 31/506; A61P 25/28; G01N 33/6896; G01N 2800/2821; G01N 2800/52

(57) ABSTRACT

The disclosure relates to lemborexant, a dual orexin receptor antagonist, and compositions and methods for use in treatment of Alzheimer's disease (AD), e.g., in a subject who has AD or who is at risk for developing AD.

### CROSS REFERENCE TO RELATED APPLICATIONS

This application claims priority to U.S. Provisional Application No. 63/376,949, filed Sep. 23, 2023, and U.S. Provisional Application No. 63/382,278, filed Nov. 3, 2023, the entire contents of which incorporated herein by reference in their entirety.

### FIELD OF DISCLOSURE

Described herein are compositions and methods relating to lemborexant, a dual orexin receptor antagonist, for use in treatment of neurological disorders such as Alzheimer's disease (AD).

### BACKGROUND

Alzheimer's disease (AD) is a progressive, neurodegenerative disorder of unknown etiology and the most common form of dementia among older people. In 2006, there were 26.6 million cases of AD in the world (range: 11.4-59.4 million) (Brookmeyer, R., et al., Forecasting the global burden of Alzheimer's Disease. Alzheimer Dement. 2007; 3:186-91), while there were more than 5 million people in the United States reportedly living with AD (Alzheimer's Association, Alzheimer's Association report, 2010 Alzheimer's disease facts and figures. Alzheimer Dement. 2010; 6:158-94). By the year 2050, the worldwide prevalence of AD is predicted to grow to 106.8 million (range: 47.2 221.2 million), while in the United States alone the prevalence is estimated to be 11 to 16 million. (Brookmeyer, supra, and 2010 Alzheimer's disease facts and figures, supra).

The disease generally involves a global decline of cognitive function that progresses slowly and leaves end-stage subjects bedridden. AD subjects typically survive for only 3 to 10 years after symptom onset, although extremes of 2 and 20 years are known. (Hebert, L. E., et al., Alzheimer disease in the U.S. population: prevalence estimates using the 2000 census. Arch Neurol. 2003; 60:1119-1122.) AD is the seventh leading cause of all deaths in the United States and the fifth leading cause of death in Americans older than the age of 65 years, despite the fact that mortality due to AD is greatly underestimated because death certificates rarely attribute the cause of death to AD.

AD represents a significant economic burden across industrialized countries with a substantial impact on healthcare systems and the public purse as well as on subjects and their families. In the United States alone, total payments for 2010 were estimated at $172 billion, including $123 billion for Medicare and Medicaid.

Histologically, the disease is characterized by neuritic plaques, found primarily in the association cortex, limbic system and basal ganglia. The major constituent of these plaques is amyloid beta peptide (Aβ). Aβ exists in various conformational states—monomers, oligomers, protofibrils, and insoluble fibrils. Details of the mechanistic relationship between onset of Alzheimer's disease and Aβ production is unknown. However, some anti-Aβ antibodies are undergoing clinical study now as potential therapeutic agents for Alzheimer's disease.

In addition to neuritic plaques, the disease is also characterized by tau aggregation and hyperphosphorylation, increased immune response, neuronal degeneration, synaptic loss, and eventual cognitive dysfunction, dementia, and death.

Insomnia has been implicated as a risk factor for AD. Historically, insomnia has been treated with various medicaments, including doxepin, a tricyclic antidepressant (TCA), and dual orexin receptor antagonists (also called DORAs) such as suvorexant or lemborexant. While the literature has suggested a link between sleep dysregulation and risk of AD, a direct link has not been established. Illustrating the need to further elucidate the link between insomnia and AD, data disclosed herein suggests that at least two such sleep medications, doxepin and lemborexant, have differential effects on AD pathology, although both mediate sleep.

Lemborexant has been approved for the treatment of adult patients with insomnia, characterized by difficulties with sleep onset and/or sleep maintenance. Lemborexant and methods of using it are disclosed in, e.g., U.S. Pat. Nos. 11,026,944 and 11,096,941, the contents of which are herein incorporated by reference.

Lemborexant has the following structure:

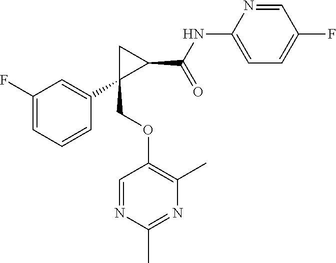 [SMILES: Cc1ncc(OC[C@@]2(c3cccc(F)c3)C[C@H]2C(=O)Nc2ccc(F)cn2)c(C)n1]

and is also known as (1R,2S)-2-(((2,4-dimethylpyrimidin-5-yl)oxy)methyl)-2-(3-fluorophenyl)-N-(5-fluoropyridin-2-yl)cyclopropanecarboxamide or (1R,2S)-2-(((2,4-dimethylpyrimidin-5-yl)oxy)methyl)-2-(3-fluorophenyl)-N-(5-fluoropyridin-2-yl)cyclopropane-1-carboxamide.

Accordingly, a need remains for improved treatment of AD, including early intervention before the development of irreversible symptoms of the disease. The disclosure herein surprisingly demonstrates that lemborexant may be used in such treatment.

### SUMMARY

One aspect of the present disclosure relates to a method for treating Alzheimer's disease (AD) in a subject who has AD or is at risk of developing AD, comprising administering to the subject a therapeutically effective amount of lemborexant, a pharmaceutically acceptable salt thereof, or a solvate thereof, thereby treating AD.

In some embodiments, treating AD comprises reducing and/or slowing cognitive decline. In some embodiments, treating AD comprises affecting a change (e.g., slowing, delaying, or reducing) in at least one marker of AD pathology.

In some embodiments, the marker is a level of phosphorylation of tau, neurodegeneration, a change in microglial response, and/or presence of Aβ plaques. In some embodiments, the marker is present in a brain region in the subject. In some embodiments, the brain region is the hippocampus, somatomotor cortex, somatosensory cortex, piriform cortex, and/or entorhinal cortex. In some embodiments, the marker is detected in a body fluid of the subject. In some embodiments, the body fluid is blood or cerebrospinal fluid (CSF).

In some embodiments, the subject does not show signs of dementia and/or cognitive impairment. In some embodiments, the subject has mild cognitive impairment or mild dementia.

In some embodiments, the subject is amyloid positive. In some embodiments, the subject is at risk for further Aβ accumulation. In some embodiments, the subject is an ApoE4 carrier. In some embodiments, the subject has intermediate levels of amyloid PET (e.g., 20-40 centiloids). In some embodiments, the subject has elevated levels of amyloid PET (e.g., >40 centiloids).

In some embodiments, the subject has been diagnosed with AD, based on brain imaging, cognitive function, and/or biomarker criteria. In some embodiments, the subject has early AD. In some embodiments, the subject has pre-AD.

An aspect of the present disclosure relates to a method of reducing or maintaining tau (e.g., reducing or maintaining a level of tau relative to the level before the start of treatment, or delaying tau accumulation, tau phosphorylation, and/or tau spreading, or slowing a rate of any of these) in a subject having AD or at risk of developing AD, comprising administering to the subject a therapeutically effective amount of lemborexant, wherein the therapeutically effective amount is sufficient to reduce or maintain tau in the subject.

In some embodiments, the subject is amyloid negative. In some embodiments, the tau levels are reduced or maintained relative to a reference. In some embodiments, the method comprises reducing and/or delaying tau accumulation and/or tau spreading, and/or slowing a rate thereof, as compared to a reference. In some embodiments, the reference is a baseline measurement from the subject prior to treatment. In some embodiments, the reference is a baseline measurement from a control subject. In some embodiments, the reference is a measurement from a control subject administered a placebo.

In some embodiments, the method comprises altering tau in a brain region of the subject. In some embodiments, the method comprises altering the tau PET signal in a brain region of the subject. In some embodiments, the brain region is the hippocampus, entorhinal cortex, and/or the piriform cortex. In some embodiments, the method comprises reducing tau in a body fluid of the subject. In some embodiments, the body fluid is blood or CSF.

In some embodiments, the tau is total tau. In some embodiments, the tau is an insoluble tau. In some embodiments, the tau is aggregated tau. In some embodiments, the tau is a phosphorylated form of tau (phospho-tau). In some embodiments, the phopho-tau is phosphorylated on one or more of T181, T217, S202, S205, or T231.

In some embodiments, the method comprises altering a ratio of phopho-tau to total tau. In some embodiments, the ratio of phospho-tau to total tau is reduced compared to the ratio of CSF phospho-tau to total tau of the subject prior to administration of lemborexant. In some embodiments, the ratio of phospho-tau to total tau is maintained within 10% of the ratio of phospho-tau to total tau of the subject prior to the administration of lemborexant. In some embodiments, the method comprises increasing a rate of dephosphorylation of phospho-tau. In some embodiments, the method comprises reducing a rate of phosphorylation of tau. In some embodiments, the method comprises reducing or maintaining tau within 48 hours of administration of a first dose of lemborexant. In some embodiments, the method comprises reducing phospho-tau in the hippocampus, entorhinal cortex, and/or piriform cortex.

Another aspect of the present disclosure relates to a method of altering neurodegeneration (e.g., reducing and/or delaying neurodegeneration, and/or slowing a rate thereof) in a subject having AD or at risk of developing AD, comprising administering to the subject a therapeutically effective amount of lemborexant, a pharmaceutically acceptable salt thereof, or a solvate thereof, wherein the therapeutically effective amount is sufficient to alter neurodegeneration in the subject.

In some embodiments, the subject is amyloid negative. In some embodiments, altering neurodegeneration comprises reducing and/or delaying neurodegeneration, and/or slowing a rate thereof, as compared to a reference. In some embodiments, the neurodegeneration is altered relative to a reference. In some embodiments, the reference is a baseline measurement from the subject prior to treatment. In some embodiments, the reference is a baseline measurement from a control subject. In some embodiments, the reference is a measurement from a control subject administered a placebo.

In some embodiments, the neurodegeneration is characterized by a loss of at least one of cortical thickness and hippocampal volume. In some embodiments, altering neurodegeneration comprises maintaining or slowing a loss reduction of cortical thickness and/or hippocampal volume. In some embodiments, the neurodegeneration is characterized by a loss of at least one of pyramidal neurons in the cortex, pyramidal neurons in the hippocampus, or granule cells in the hippocampus. In some embodiments, altering neurodegeneration comprises maintaining or reducing loss of pyramidal neurons and/or granule cells. In some embodiments, altering neurodegeneration comprises reducing a rate of neurodegeneration. In some embodiments, altering neurodegeneration comprises altering a neurofilament light chain (NfL) level. In some embodiments, the method comprises altering the NfL levels in the blood and/or CSF of the subject.

A further aspect of the present disclosure relates to a method of altering Aβ plaques (e.g., reducing or delaying formation of Aβ plaques, or slowing a rate of growth thereof) in a subject having AD or at risk of developing AD, comprising administering to the subject a therapeutically effective amount of lemborexant, a pharmaceutically acceptable salt thereof, or a solvate thereof, wherein the therapeutically effective amount is sufficient to alter Aβ plaques in the subject.

In some embodiments, the Aβ plaques are altered relative to a reference. In some embodiments, altering Aβ plaques comprises reducing and/or delaying formation of Aβ plaques, and/or slowing a rate thereof, as compared to a reference. In some embodiments, the reference is a baseline measurement from the subject prior to treatment. In some embodiments, the reference is a baseline measurement from a control subject. In some embodiments, the reference is measurement from a control subject administered a placebo.

In some embodiments, the Aβ plaques are fibrillar plaques. In some embodiments, the Aβ plaques all plaques (e.g., including diffuse plaques). In some embodiments, altering Aβ plaques comprises reducing the growth of Aβ plaques. In some embodiments, the method comprises reducing growth of Aβ plaques in the hippocampus of the subject, somatomotor cortex, the somatosensory cortex, and/or the piriform cortex of the subject.

In some embodiments, altering Aβ plaques comprises altering an amyloid PET signal obtained from a brain region of the subject. In some embodiments, altering Aβ plaques corresponds to a reduction in the concentration of Aβ in the subject's CSF.

In some embodiments, the Aβ is Aβ38, Aβ40, and/or Aβ42. In some embodiments, altering Aβ plaques within 48 hours of administration of a first dose of lemborexant.

In some embodiments, the subject does not show signs of dementia and/or cognitive impairment. In some embodiments, the subject has mild cognitive impairment or mild dementia.

In some embodiments, the subject is at risk for further Aβ accumulation. In some embodiments, the subject is an ApoE4 carrier. In some embodiments, the subject has intermediate levels of amyloid PET (e.g., 20-40 centiloids). In some embodiments, the subject has elevated levels of amyloid PET (e.g., >40 centiloids).

In some embodiments, the subject has early-stage AD. In some embodiments, the subject has pre-AD.

One aspect of the present disclosure relates to a method of modulating a microglial response in a subject having Alzheimer's disease (AD) or at risk of developing AD, comprising administering to the subject a therapeutically effective amount of lemborexant, a pharmaceutically acceptable salt thereof, or a solvate thereof, wherein the therapeutically effective amount is sufficient to modulate the microglial response in the subject.

In some embodiments, modulating the microglial response comprises modulating expression of at least one microglial marker. In some embodiments, the microglial marker is a general microglial marker. In some embodiments, the general microglial marker is Iba1, Clec7a, or CD68. In some embodiments, the microglial marker is a homeostatic microglial marker. In some embodiments, the homeostatic microglial marker is TMEM119 or P2RY12. In some embodiments, modulating the microglial response comprises modulating activity of phagocytic microglia.

In some embodiments, the subject has mild cognitive impairment or mild dementia. In some embodiments, the subject does not show signs of dementia and/or cognitive impairment.

In some embodiments, the subject is amyloid negative. In some embodiments, the subject has tau pathology. In some embodiments, the subject has neurodegeneration in a brain region. In some embodiments, the brain region is the hippocampus, the entorhinal cortex, and/or the piriform cortex. In some embodiments, the brain region is the CA1 region, the CA2 region, the CA3 region, or the dentate gyrus in the hippocampus.

In some embodiments, modulating the microglial response comprises modulating a response in microglia associated with degenerating neurons. In some embodiments, modulating the microglial response comprises reducing expression of at least one general microglial marker. In some embodiments, the general microglial marker is Iba1, CD68, or Clec7a. In some embodiments, modulating the microglial response comprises increasing expression of at least one homeostatic microglial marker. In some embodiments, the homeostatic microglial marker is TMEM119 or P2RY12.

In some embodiments, the subject has Aβ plaques. In some embodiments, the Aβ plaques are fibrillar Aβ plaques. In some embodiments, the subject is at risk for further Aβ accumulation. In some embodiments, the subject is an ApoE4 carrier. In some embodiments, the subject has intermediate levels of amyloid PET (e.g., 20-40 centiloids). In some embodiments, the subject has elevated levels of amyloid PET (e.g., >40 centiloids).

In some embodiments, the subject has early-stage AD. In some embodiments, the subject has pre-AD.

In some embodiments, the Aβ plaques are present in the hippocampus, the somatomotor cortex, the somatosensory cortex, and/or the piriform cortex.

In some embodiments, modulating the microglial response comprises modulating a response in microglia associated with Aβ plaques. In some embodiments, modulating the microglial response comprises increasing expression of a general microglial marker. In some embodiments, the general microglial marker is Iba1, Clec7a, or CD68. In some embodiments, modulating microglial response comprises increasing phagocytosis of Aβ plaques by phagocytic microglia. In some embodiments, modulating the microglial response comprises reducing expression of a homeostatic microglial marker. In some embodiments, the homeostatic microglial marker is TMEM119 or P2RY12.

In some embodiments of any of the methods disclosed herein, the therapeutically effective amount of lemborexant administered to the subject is in a range of 5 mg to 50 mg per day. In some embodiments, the therapeutically effective amount of lemborexant administered to the subject is in a range of 10 mg to 30 mg per day.

In some embodiments, the therapeutically effective amount of lemborexant administered to the subject is selected from 5 mg, 7.5 mg, 10 mg, 12.5 mg, 15 mg, 17.5 mg, 20 mg, 22.5 mg, 25 mg, 27.5 mg and 30 mg per day.

In some embodiments, the therapeutically effective amount of lemborexant administered to the subject is 20-25 mg per day. In some embodiments, one dose of 25 mg of lemborexant is administered to the subject once per day.

In some embodiments, lemborexant is administered at a first dose for a first period, a second dose for a second period, and optionally, at a third dose for a third period. In some embodiments, each of the first period, the second period, and the third period are 1 week. In some embodiments, the first dose is lower than the second dose, and optionally, the second dose is lower than the third dose.

In some embodiments, the first dose is 5 mg of lemborexant once per day, the second dose is 10 mg of lemborexant once per day, and, optionally, the third dose is 20-25 mg of lemborexant once per day. In some embodiments, the first dose is 5 mg or 7.5 mg lemborexant once per day, the second dose is 10 mg, 12.5 mg, 15 mg, or 17.5 mg of lemborexant once per day, and the third dose is 20 mg, 22.5 mg, 25 mg, 27.5 mg or 30 mg of lemborexant once per day.

In some embodiments, the first dose is higher than the second dose, and optionally, the second dose is higher than the third dose. In some embodiments, the first dose is 20-25 mg of lemborexant once per day, the second dose is 10 mg of lemborexant once per day, and, optionally, the third dose is 5 mg of lemborexant once per day. In some embodiments, the first dose is 20 mg, 22.5 mg, 25 mg, 27.5 mg or 30 mg of lemborexant once per day, the second dose is 10 mg, 12.5 mg, 15 mg, or 17.5 mg of lemborexant once per day, and, optionally, the third dose is 5 mg or 7.5 mg of lemborexant once per day.

In some embodiments of any of the methods disclosed herein, comprising administering lemborexant to the subject for at least 6 months. In some embodiments, the method comprises administering lemborexant to the subject for at least 9 months, at least 12 months, or at least 15 months. In some embodiments, the method comprises administering lemborexant to the subject for at least 18 months. In some embodiments, the method comprises administering lemborexant to the subject for at least 24 months, 30 months, or 36 months.

Another aspect of the present disclosure relates to a method of selecting a subject having Alzheimer's disease (AD) or at risk of developing AD for treatment with lemborexant, a pharmaceutically acceptable salt thereof, or a solvate thereof, comprising: (a) obtaining from the subject a measurement of at least one of tau phosphorylation, tau aggregation, neurodegeneration, Aβ plaque burden, microglial response, and biomarker expression; (b) comparing the measurement from the subject to a measurement from a reference; and (c) selecting the subject for treatment with lemborexant if the measurement from the subject differs from the measurement from the reference.

In some embodiments, the subject has mild cognitive impairment or mild dementia. In some embodiments, the subject does not show signs of dementia and/or cognitive impairment.

In some embodiments, the subject is at risk for Aβ accumulation. In some embodiments, the subject is an ApoE4 carrier. In some embodiments, the subject has intermediate levels of amyloid PET (e.g., 20-40 centiloids). In some embodiments, the subject has elevated levels of amyloid PET (e.g., >40 centiloids).

In some embodiments, the subject has early-stage AD. In some embodiments, the subject has pre-AD.

In some embodiments, the subject has been diagnosed with AD, based on brain imaging, cognitive function, and/or biomarker criteria. In some embodiments, obtaining at least one measurement comprises obtaining data from a brain scan of the subject and/or obtaining data from a biological sample from the subject. In some embodiments, the data from the brain scan indicates a level of tau phosphorylation, tau aggregation, Aβ plaque burden, and/or microglial response. In some embodiments, the biological sample is a body fluid. In some embodiments, the body fluid is cerebrospinal fluid (CSF), blood, or saliva.

In some embodiments, the reference is a control. In some embodiments, the reference is a measurement from a control subject administered a placebo.

In some embodiments, the control does not have AD. In some embodiments, the measurement from the subject is higher than the measurement from the control who does not have AD. In some embodiments, the measurement from the subject is lower than the measurement from the control who does not have AD.

In some embodiments, the control has AD. In some embodiments, the measurement from the subject is comparable to or higher than the measurement from the control who has AD. In some embodiments, the measurement from the subject is comparable to or lower than the measurement from the control who has AD.

In some embodiments, the measurement of tau phosphorylation comprises a measurement of phosphorylation on one more of T181, T217, S202, S205, or T231. In some embodiments, the measurement of tau aggregation comprises a measurement of insoluble tau aggregates (e.g., neurofibrillary tangles (NFTs)).

In some embodiments, the measurement of neurodegeneration comprises a measurement of cortical thickness and/or hippocampal volume or a measurement of loss of pyramidal neurons or granule neurons.

In some embodiments, the measurement of Aβ plaque burden comprises a measurement of Aβ plaque volume and/or growth of Aβ plaque volume. In some embodiments, the measurement of Aβ plaque burden comprises a measurement of amyloid PET signal in a brain region of the subject or a measurement of Aβ in the CSF of the subject.

In some embodiments, the measurement of microglial response is a change in the expression of at least one microglial marker. In some embodiments, the microglial marker is Iba1, Clec7a, CD68, TMEM119, or P2RY12. In some embodiments, the measurement of microglial response is a measurement of phagocytosis by microglia.

A further aspect of the present disclosure relates to a method of monitoring treatment efficacy in a subject having Alzheimer's disease (AD) or at risk of developing AD, comprising: (a) obtaining from the subject a first measurement of at least one of tau phosphorylation, tau aggregation, neurodegeneration, Aβ plaque burden, microglia function, and biomarker expression; (b) administering to the subject a dose of lemborexant, a pharmaceutically acceptable salt thereof, or a solvate thereof; (c) obtaining from the subject a second measurement of at least one of tau phosphorylation, tau aggregation, neurodegeneration, Aβ plaque burden, microglia function, and biomarker expression; and (d) comparing the second measurement from the subject to the first measurement from the subject, wherein a difference between the first measurement and the second measurement indicates effective treatment with lemborexant.

Another aspect of the present disclosure relates to a method of treating a subject having Alzheimer's disease (AD) or at risk of developing AD, comprising: (a) obtaining from the subject a first measurement of at least one of tau phosphorylation, tau aggregation, neurodegeneration, Aβ plaque burden, microglial response, and biomarker expression; (b) administering to the subject a first dose of lemborexant, a pharmaceutically acceptable salt thereof, or a solvate thereof; (c) obtaining from the subject a second measurement of at least one of tau phosphorylation, tau aggregation, neurodegeneration, Aβ plaque burden, microglia function, and biomarker expression; (d) comparing the second measurement from the subject to the first measurement from the subject, and (e) administering a second dose of lemborexant if the first measurement differs from the second measurement.

In some embodiments, obtaining at least one measurement comprises obtaining data from a brain scan of the subject and/or obtaining data from a biological sample from the subject. In some embodiments, the data from the brain scan indicates a level of tau phosphorylation, tau aggregation, Aβ plaque burden, and/or microglial response. In some embodiments, the biological sample is a body fluid. In some embodiments, the body fluid is cerebrospinal fluid (CSF), blood, or saliva.

In some embodiments, the first measurement from the subject is higher than the second measurement from the subject. In some embodiments, the first measurement from the subject is lower than the second measurement from the subject.

In some embodiments, the measurement of tau phosphorylation comprises a measurement of phosphorylation of one or more of T181, T217, S202, S205, or T231. In some embodiments, the measurement of tau aggregation comprises a measurement of insoluble tau aggregates (e.g., neurofibrillary tangles (NFTs)).

In some embodiments, the measurement of neurodegeneration comprises a measurement of cortical thickness and/or hippocampal volume or a measurement of loss of pyramidal neurons or granule neurons.

In some embodiments, the measurement of Aβ plaque burden comprises a measurement of Aβ plaque volume and/or growth of Aβ plaque volume. In some embodiments, the measurement of Aβ plaque burden comprises a measurement of amyloid PET signal in a brain region of the subject or a measurement of Aβ in the CSF of the subject.

In some embodiments, the measurement of microglial response is a measure of expression of at least one microglial marker. In some embodiments, the microglial marker is Iba1, Clec71, P2RY12 or TMEM 119. In some embodiments, the measurement of microglial response is a measurement of phagocytosis by microglia. In some embodiments, the measurement of a biomarker expression is a measurement of Ifnb1, MMP2, and/or Bace1 expression.

In some embodiments, the subject is amyloid-negative. In some embodiments, the subject has Aβ plaques.

In some embodiments, the subject has mild cognitive impairment and/or mild dementia. In some embodiments, the subject does not show signs of dementia and/or cognitive impairment.

In some embodiments, the subject is at risk for further Aβ accumulation. In some embodiments, wherein the subject is an ApoE4 carrier. In some embodiments, wherein the subject has intermediate levels of amyloid PET (e.g., 20-40 centiloids). In some embodiments, wherein the subject has elevated levels of amyloid PET (e.g., >40 centiloids).

In some embodiments, the subject has early-AD. In some embodiments, the subject has pre-AD.

### BRIEF DESCRIPTION OF THE DRAWINGS

FIG. 1 shows a study schema for a clinical trial. FIG. 1 depicts an overview of the study design with a habitual bedtime of 22:00.

FIGS. 2A-H depicts data from the study of Example 2. FIG. 2A shows a schematic illustration of the study design. Electroencephalography (EEG) analyses shows of percentage time spent in sleep; FIG. 2B shows NREM sleep, FIG. 2C shows REM sleep, and FIG. 2D shows awake in vehicle (Veh) or Lemborexant (Lem)-treated E4 (n=10 mice/treatment group) and P301S/E4 mice (n=8 mice/treatment group. Two-way ANOVA, Tukey's post hoc comparison. FIG. 2E shows representative spectrograms of EEG and electromyography (EMG) analyses illustrating NREM, REM and wake patterns in vehicle-treated P301S/E4 and FIG. 2F shows Lemborexant-treated P301S/E4 mice. FIG. 2G shows a time-course analysis of percentage sleep observed over time starting at vehicle or Lemborexant treatment until 24 hours post-oral gavage in E4 mice (n=10 mice/treatment group) and FIG. 2H shows results in P301S/E4 mice (n=8 mice/treatment group). White and dark bars note light phase and dark phase, respectively. Data represent mean±SEM; *p<0.05, **p<0.001, ***p<0.0001.

FIG. 3 depicts data from the study of Example 2. FIG. 3A shows representative images of AT8-stained phosphorylated tau at both serine 202 and threonine 205. Top panel show hippocampus and bottom show entorhinal and piriform cortices. Scale bar—500 μm. FIG. 3B shows representative images of MC1-stained tau. Scale bar—500 μm. FIG. 3C shows quantification of percentage AT8 covered hippocampus (nE4 and nP301S/E4=16-17 mice/treatment group), and FIG. 3D shows entorhinal/piriform cortices (nE4=16-18 mice/treatment group; nP301S/E4=15-19 mice/treatment group). FIG. 3E shows percentage of MC1-stained hippocampus (nE4=18 mice/treatment group; nP301S/E4=16-17 mice/treatment group), and FIG. 3F shows entorhinal/piriform cortices (nE4=16-17 mice/treatment group; nP301S/E4=15-17 mice/treatment group). FIG. 3G shows representative images of cresyl violet-stained brains used for volumetric analysis. Scale bar—1 mm. FIG. 3H shows quantification of hippocampus volume and, FIG. 3I shows piriform cortex volume. FIG. 3J shows plasma neurofilament light chain (NfL) levels measured by SIMOA (nE4 and nP301S/E4=16-20 mice/treatment group. Data represent mean±SEM; Two-way ANOVA, Tukey's post hoc test; *p<0.05, **p<03.001, ***p<0.0001.

FIGS. 4A-R depicts data from the study of Example 2. FIG. 4A shows representative images of IBA1 (green), CD68 (red) and DAPI (blue) co-stained microglia of the CA3 region in hippocampus. FIG. 4B shows representative images of TMEM119 (yellow) and DAPI (blue) stained microglia in hippocampal CA3 region. FIG. 4C shows quantification of percentage IBA1 covered, and FIG. 4D shows CD68 covered CA3. FIG. 4E shows representative images of Clec7a (red) and DAPI (blue) stained microglia. FIG. 4F shows quantification of percentage TMEM119 covered, and FIG. 4 G shows Clec7a covered CA3. FIG. 4H shows representative images of ApoE (green) and GFAP (red) costained astroglia. FIG. 4I shows quantification of percentage ApoE colocalized with GFAP, or FIG. 4J shows IBA1 in CA3. FIG. 4K shows representative images of ApoE (green) costained with IBA1 (magenta) positive microglia. FIG. 4L shows the percentage of Iba1-covered piriform/entorhinal cortex in lemborexant-treated mice as compared to vehicle-treated control mice. FIG. 4M shows the percentage of Iba1-covered dentate gyrus in lemborexant-treated mice as compared to vehicle-treated control mice. FIG. 4N shows the percentage of TMEM119-covered piriform/entorhinal cortex in lemborexant-treated mice as compared to vehicle-treated control mice. FIG. 4O shows the percentage of TMEM119-covered dentate gyrus in lemborexant-treated mice as compared to vehicle-treated control mice. FIG. 4P shows the percentage of CD68-covered dentate gyrus in lemborexant-treated mice as compared to vehicle-treated control mice. FIG. 4Q shows the percentage of CD68-covered piriform/entorhinal cortex in lemborexant-treated mice as compared to vehicle-treated control mice. FIG. 4R shows a representative image of the CA1/CA2 region stained for GFAP, and FIG. 4S shows plots of the percentage of GFAP-covered CA1/2, FIG. 4T shows plots of the dentate gyrus, and FIG. 4U shows plots of the piriform/entorhinal cortex. Scale bar −50 μm. nE4=15-19 mice/treatment group; nP301S/E4=16-19 mice/treatment group. Data represent mean±SEM; Two-way ANOVA, Tukey's post hoc test; *p<0.05, **p<0001, ***p<0.0001.

FIG. 5 depicts data from the study of Example 2. FIG. 5A shows a volcano plot comparing differentially regulated genes in P301S/E4 mice treated with vehicle versus Lemborexant. Cut off value for significance is set at two-fold Log fold change. nE4 and nP301S/E4=10 mice/treatment group. FIG. 5B shows a GO term analysis of genes significantly changed in P301S/E4 mice treated with vehicle versus Lemborexant. FIG. 5C shows a heatmap illustrating all differentially expressed genes in P301S/E4 vehicle versus lemborexant that reached significance of Log 10 adjusted p value <0.05. FIGS. 5D and 5E show representative images of VGLUT1 and PSD95-stained synapses in CA3. Scale bar—50 μm. FIG. 5F shows quantification of percentage VGLUT1 puncta in CA3, and FIG. 5G shows results in piriform cortex. FIG. 5H shows quantification of percentage PSD95 puncta in CA3, and FIG. 5I shows results in piriform cortex. nE4=16-19 mice/treatment group; nP301S/E4=16-19 mice/treatment group. Data represent mean±SEM; two-tailed unpaired T-test; *p<0.05, **p<0.005, ***p<0,0001.

FIG. 6 depicts data from the study of Example 2. FIG. 6A shows a time course analysis of percentage sleep in E4 mice (n^{E4}=7-9 mice/treatment group), and FIG. 6B shows results in P301S/E4 mice (n^{P301S/E4}=8-10 mice/treatment group). White and dark bars note light phase and dark phase, respectively. FIG. 6C shows the percentage of time spent asleep in light or dark phase in E4 and P301S/E4 mice (n=10 mice/genotype and treatment group). FIG. 6D shows sleep bout length and FIG. 6E shows wake bout length in light or dark phase in E4 and P301S/E4 mice (n=10 mice/genotype and treatment group). s—seconds. Data represent mean±SEM; Two-way ANOVA, Tukey's post hoc test; *p<0.05, **p<0.001, ***p<0.0001.

FIG. 7 depicts data from the study of Example 2. Phosphorylated tau (pTau) was quantified in RAB (FIG. 7A), RIPA (FIG. 7B), and formic acid (FA) (FIG. 7C) fractions by ELISA (nE4 and nP301S/E4=18-20 mice/treatment group). Total tau (tTau) was quantified in RAB (FIG. 7D), RIPA (FIG. 7E), and FA (FIG. 7F) fractions by ELISA (nE4=18-20 and nP301S/E4=18-20 mice/treatment group). In FIG. 7G, the top panel illustrates representative images of hippocampus including granule cell layer, and the bottom panel show piriform cortex including pyramidal cell layer. FIG. 7H shows the volumetric analysis of cresyl violet-stained hippocampus, FIG. 7I shows the hemibrain subtracted by ventricle, FIG. 7J shows the pyramidal cell layer of the piriform cortex, and FIG. 7K shows the hippocampal granule cell layer. (nE4 and nP301S/E4=17-19 mice/treatment group). Scale bar—500 μm. Data represent mean±SEM; Two-way ANOVA, Tukey's post hoc test; *p<0.05, **p<0.001, ***p<0.0001.

FIG. 8 depicts data from the study of Example 2. FIG. 8A shows representative images of DAPI, IBA1 and P2RY12-costained microglia in the CA3. FIG. 8B shows percentage P2RY12 covered CA3 quantified and FIG. 8C shows results in the dentate gyrus. nE4 and nP301S/E4=16-19 mice/treatment group. Scale bar—50 μm. Data represent mean±SEM; Two-way ANOVA, Tukey's post hoc test; *p<0.05, **p<0.001, ***p<0.0001. FIG. 8C shows the percentage of P2RY12-covered dentate gyrus. FIG. 8D shows the percentage of P2RY12-covered piriform/entorhinal cortex.

FIG. 9, from the study in Example 3, shows changes in sleep in APP/PS1dE9 mice that were administered doxepin or lemborexant. FIG. 9A is a schematic of the experimental design. FIG. 9B, FIG. 9C, and FIG. 9D show the effects on total sleep, light phase sleep, and dark phase sleep in mice treated with doxepin, lemborexant (10 mg or 30 mg), or a vehicle. FIG. 9E shows the percentage of sleep as a function of Zeitgeber Time (ZT0=light on).

FIG. 10, from the study in Example 3, shows the fibrillar amyloid plaque burden in APP/PS1dE9 mice that were administered doxepin or lemborexant. FIG. 10A is a schematic for the timing of treatment of the mice. FIG. 10B shows representative images of brain sections stained with X34, which labels fibrillar amyloid plaques. FIG. 10C shows quantification of plaque burden (% area X34 staining) in different brain regions. Error bars indicate mean±SEM, each dot is a mouse. P values from 1-way ANOVA are shown.

FIG. 11, from the study of Example 3, shows the total amyloid plaque burden in APP/PS1dE9 mice treated with doxepin or lemborexant, as shown in FIG. 10A. FIG. 11A shows representative brain sections stained for total amyloid plaques burden using the anti-Ap antibody HJ3.4. FIG. 11B is a quantification of plaque burden (% area HJ3.4 staining) in different brain regions. Error bars indicate mean±SEM, each dot is a mouse. P values from 1-way ANOVA are shown.

FIG. 12, from the study of Example 3, shows APP processing/cleavage in APP/PS1dE9 mice treated with doxepin or lemborexant. FIG. 12A shows the results of a representative Western blot for full-length APP and APP C-terminal fragments (CTF-α and -β). Beta-tubulin is shown as a loading control. FIG. 12B shows the quantification of band intensity. Error bars indicate mean SEM, each dot is a mouse. P values from 1-way ANOVA are shown.

FIG. 13, from the study of Example 3, shows peri-plaque microglial clustering in APP/PS1dE9 mice treated with doxepin or lemborexant. FIG. 13A shows representative images of samples stained for plaques (X34) and microglia (Iba1). The volume of microglia around each plaque was calculated from Z-stacks of confocal images using Imaris software. FIG. 13B shows quantification of plaque volume (to indicate that similar sized plaques were quantified across conditions), and peri-plaque Iba1 volume. Error bars indicate mean±SEM, each dot is a mouse. P values from 1-way ANOVA are shown.

FIG. 14, from the study of Example 3, shows peri-plaque microglial CD68 expression in APP/PS1dEP mice in APP/PS1dEP mice treated with doxepin or lemborexant. FIG. 14A show representative images of samples stained for plaques (X34), microglia (Iba1), phagosomes (CD68). FIG. 14B shows Iba1-colocalized CD68 quantified around each plaque using Imaris software. Error bars indicate mean±SEM, each dot is the average of 8-10 plaques from a single mouse. P values from 1-way ANOVA are shown. FIG. 14C shows the Iba1 volume (μm³). FIG. 14D shows co-localization of Iba1 and CD68, measured as a percentage of Iba1. FIG. 14E shows the volume of co-localized Iba1 and CD68.

FIG. 15, from the study of Example 3, shows the effects of lemborexant treatment on gene expression. Expression of transcripts encoding Ifnb1, Rab5a, and Mmp2 showed significant differences after lemborexant treatment. Data is displayed as fold change (relative to the mean for VEH). Error bars indicate mean±SEM, each dot is a single mouse. P values from 1-way ANOVA are shown.

FIG. 16, from the study of Example 3, shows microglial amyloid plaque phagocytosis in APP/PS1dEP mice treated with lemborexant. FIG. 16A shows a schematic of the experimental design. FIG. 16B shows the flow cytometry gating strategy. FIG. 160C shows methoxy-X04 (MX04) positivity in a CD45-low, CD11b+ population isolated as likely microglia. FIG. 16D is a quantification of the percentage of MX04+ microglia, p=0.0207 by a 2-tailed T-test.

FIG. 17, from the study of Example 3, shows the amyloid plaque growth in mice with pre-existing plaques. FIG. 17A is a schematic of experimental design. FIG. 17B shows representative images of MX04, thiazine Red, and overlay images. P values are shown from a 1-way ANOVA. FIG. 17C shows representative images of amyloid plaques labeled with X34, microglia labeled with IBA1, and microglial phagosome labeled with CD68. FIG. 17D shows the percentage of plaque volume growth in VEH and lemborexant-treated mice. P values are from Mann-Whitney U test, due to non-Gaussian distribution of data. FIG. 17E shows the quantification of colocalized IBA1-CD68, displayed as a percentage of total IBA1 (total microglial) area. P values are from a 1-way ANOVA. FIG. 17F shows the percentage of plaque volume growth in mice treated with lemborexant, doxepin, or a vehicle control. FIG. 17G shows the quantification of colocalized IBA1-CD68, displayed as a % of total IBA1 (total microglial) area. All graphs display mean SEM, and each dot is one mouse.

FIG. 18, from the study of Example 4, shows the effects of lemborexant or doxepin on rhythmic activity patterns in arrhythmic Bmal1 KO mice. FIG. 18A shows representative actograms. LD=12 h:12 h light dark; DD=constant dark. FIG. 18B shows a quantification of circadian locomotor behavior during different portions of the experiment (LD is indicated by the shaded area, DD+LEM is area with the shaded arrow, and DD is the rest of the recording). Data analyzed by 2-way ANOVA with Tukey post-hoc test.

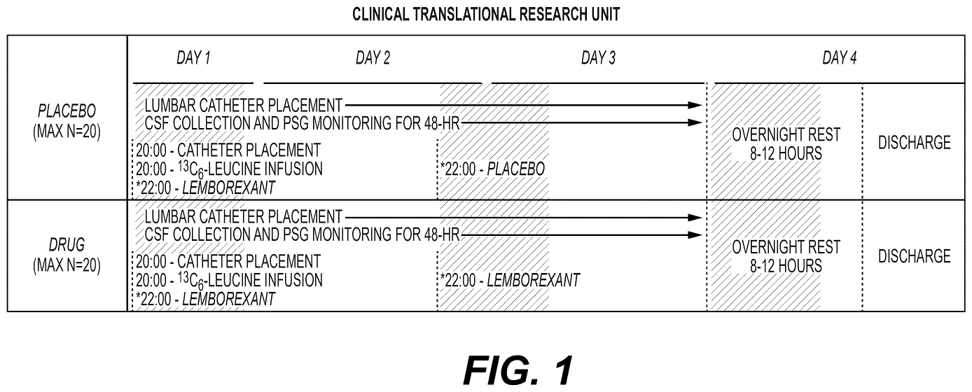

### DETAILED DESCRIPTION

### I. Definitions

The following are definitions of terms used in the present application.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B,” when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “at least one” means one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A): in yet another embodiment, to at least one, optionally including more than one, A, and at least one, optionally including more than one, B (and optionally including other elements); etc.

When a number is recited, either alone or as part of a numerical range, it should be understood that the numerical value can vary above and below the stated value by a variance of 10% of the stated value.

When a range of values is listed herein, it is intended to encompass each value and sub-range within that range. For example, “15 mg to 30 mg” is intended to encompass, for example, 15.0 mg, 15.5 mg, 16.0 mg, 16.5 mg, 17.0 mg, 17.5 mg, 18.0 mg, 18.5 mg, 19.0 mg, 19.5 mg, 20.0 mg, 20.5 mg, 21.0 mg, 21.5 mg, 22.0 mg, 22.5 mg, 23.0 mg, 23.5 mg, 24.0 mg, 24.5 mg, 25.0 mg, 25.5 mg, 26.0 mg, 26.5 mg, 27.0 mg, 27.5 mg, 28.0 mg, 28.5 mg, 29.0 mg, 29.5 mg, 30.0 mg, 15 mg to 15.5 mg, 15 mg to 16 mg, 15 mg to 17.5 mg, 17.5 mg to 21 mg, 15 mg to 28 mg, and so forth.

“Amyloid” refers to aggregates of proteins which form a fibrillar morphology. Amyloids are often formed from long, unbranched fibers that are characterized by an extended s-sheet secondary structure, which are approximately 7-13 nm in width and a few micrometers in length. Amyloids are usually extracellular, and found in vivo; in addition, the fibers bind the dye Congo Red and then show green birefringence when viewed between crossed polarizers. Amyloid-forming proteins have been identified and associated with serious diseases, including amyloid-β peptide (Aβ) with Alzheimer's disease (AD), islet amyloid polypeptide (IAPP) with diabetes type 2, and prion protein (PrP) with the spongiform encephalopathies. As used herein, “amyloid,” “amyloid beta,” “brain amyloid,” and “amyloid-β peptide (Aβ)” are used interchangeably.

Amyloid β 1-42 (Aβ42) refers to an amyloid beta monomer from amino acid 1 to 42 of the full-length protein (Table 5, SEQ ID NO:13). Amyloid β 1-40 (Aβ1-40) refers to an amyloid beta monomer from amino acid 1 to 40 of the full-length protein (Table 5, SEQ ID NO:14).

Amyloid levels from amyloid PET can be reported using the Centiloid method in “centiloid” units (CL). (Klunk W E et al. The Centiloid Project: standardizing quantitative amyloid plaque estimation by PET. Alzheimer's Dement. 2015; 11:1-15 e1-4). The Centiloid method measures a tracer on a scale of 0 CL to 100 CL, where 0 is deemed the anchor-point and represents the mean in young healthy controls and 100 CL represents the mean amyloid burden present in subjects with mild to moderate severity dementia due to AD. (Id.) As is known to one of ordinary skill in the art, centiloid thresholds may vary, for example may be refined, based on new or additional scientific information. (See, e.g., http://www.gaain.org/centiloid-project.) An elevated level of amyloid can be set relative to a baseline threshold in a healthy control determined according to methods known to a person of ordinary skill in the art.

As used herein, whether a subject is “amyloid positive” or “amyloid negative” may be determined based on whether the subject has a positive amyloid load. In some embodiments, a subject is determined to be amyloid-positive or amyloid-negative as indicated by longitudinal positron emission tomography (PET) assessment of an amyloid imaging agent uptake into the brain. In some embodiments, a subject is “amyloid negative” if the florbetapir amyloid PET SUVr negativity is below 1.17. In some embodiments, a subject is determined to be amyloid-positive or amyloid-negative by a CSF assessment of the presence of amyloid pathology using assessments of markers such as Aβ1-42 (e.g., a soluble CSF biomarker analysis), alone or in combination with another method such as PET measurement of brain amyloid. Methods for measuring Aβ38, Aβ40, and Aβ42 are known in the art, such as assays using LC MS/MS. Methods may include the PrecivityAD™ assay (see, e.g., Kirmess et al., J. Clinica Chimica Acta 519: 267-275 (2021)) and the Sysmex assay (https://www.eisai.com/news/2019/news201990.html) for measuring Aβ42 and Aβ40 in a blood or plasma sample or a CSF sample. In some embodiments, a qualitative visual read of PET scans may be used to determine amyloid positive and amyloid negative by categorizing subjects as having either “normal” or “abnormal” uptake on the basis of the PET image pattern. Readers will have been trained and certified to recognize brain PET images with abnormal or normal patterns of uptake, or the detection of amyloid is done through a semi-quantitative or quantitative approach. In some embodiments, a threshold will be set for quantitatively determining from a biomarker (e.g., serum or CSF) and/or PET scan whether an Aβ brain load indicates a subject is amyloid-positive or negative. In some embodiments, a subject is determined to be amyloid-positive or amyloid-negative by an MRI. In some embodiments, the whole brain or at least one area of the brain (for example, cortical gray matter (i.e., cortex), lateral ventricles, frontal lobe, parietal lobe, temporal lobe, occipital lobe, cingulate cortex, amygdala, piriform cortex, entorhinal cortex, hippocampus, hippocampal CA3 (pyramidal neurons), and/or hippocampal dentate gyrus (granule cell neurons)) is/are analyzed by MRI.

In some embodiments, a subject is determined to be amyloid-positive or amyloid-negative by retinal amyloid accumulation. In some embodiments, a subject is determined to be amyloid-positive or amyloid-negative by behavioral/cognitive phenotypes.

The term “tau protein” or “tau” encompasses all tau isoforms, whether full-length, truncated, or post-translationally modified. In many animals, including but not limited to humans, non-human primates, rodents, fish, cattle, frogs, goats, and chicken, tau is encoded by the gene MAPT. In humans, there are six isoforms of tau that are generated by alternative splicing of exons 2, 3, and 10 of MAPT. These isoforms range in length from 352 to 441 amino acids. Exons 2 and 3 encode 29-amino acid inserts each in the N-terminus (called N), and full-length human tau isoforms may have both inserts (2N), one insert (1N), or no inserts (0N). All full-length human tau isoforms also have three repeats of the microtubule binding domain (called R). Inclusion of exon 10 at the C-terminus leads to inclusion of a fourth microtubule binding domain encoded by exon 10. Hence, full-length human tau isoforms may be comprised of four repeats (4R) of the microtubule binding domain (exon 10 included) or three repeats (3R) of the microtubule binding domain (exon 10 excluded). Human tau may or may not be post-translationally modified. For example, it is known in the art that tau may be phosphorylated, ubiquinated, glycosylated, and glycated. Accordingly, the term “human tau” encompasses the (2N, 3R), (2N, 4R), (1N, 3R), (1N, 4R), (0N, 3R), and (0N, 4R) isoforms, isoforms that are N- and/or C-terminally truncated species thereof, and all post-translationally modified isoforms. Alternative splicing of the gene encoding tau similarly occurs in other animals. In animals where the gene is not identified as MAPT, a homolog may be identified by methods well known in the art.

Phosphorylation of specific amino acids (i.e. “sites” or “residues”) in tau results in phosphorylated tau (p-tau) isoforms. Phosphorylation may occur at different residues, such as T111, S113, T181, S199, S202, S208, T153, T175, T205, S214, T217, and T231.

The term “p-tau” encompasses all phosphorylated tau (p-tau) isoforms, such as but not limited to p-tau181, p-tau217 and p-tau231.

A disease associated with tau deposition in the brain may be referred to as a “tauopathy” or “tau pathology.” A clinical sign of a tauopathy may be aggregates of tau in the brain, including but not limited to neurofibrillary tangles. Other methods can be used to detect in a subject or measure tau phosphorylation at one or more amino acid residue and optionally total tau. For example, tau can be purified from blood or cerebrospinal fluid (CSF) obtained from a subject. CSF may be obtained by lumbar puncture.

Methods to measure phosphorylation of tau include high-resolution mass spectrometry. Suitable types of mass spectrometers are known in the art. These include, but are not limited to, quadrupole, time-of-flight, ion trap and Orbitrap, as well as hybrid mass spectrometers that combine different types of mass analyzers into one architecture (e.g., Orbitrap Fusion™ Tribrid™ Mass Spectrometer from ThermoFisher Scientific). Other methods for measuring p-tau (phosphor-tau) and t-tau (total-tau) are known in the art, such as assays using Liquid Chromatography with tandem mass spectrometry (LC-MS-MS). The measurement of p-tau and t-tau can also be determined by positron emission tomography (PET) using radiotracers. The whole brain or at least one area of the brain (cortical gray matter (i.e., cortex), frontal lobe, parietal lobe, temporal lobe, occipital lobe, cingulate cortex, amygdala, piriform cortex, entorhinal cortex, hippocampus) can be analyzed by PET.

As used herein, “relative to placebo” refers to a comparison of a biomarker (p-tau, Aβ, etc.) between the same biomarker of a subject being administered lemborexant and another subject being administered a placebo (a substance that has no therapeutic effect).

As used herein, “relative to baseline” refers to a comparison of a biomarker (p-tau, Aβ, etc.) between the same biomarker of a subject being administered lemborexant and the same subject prior to treatment with lemborexant.

As used herein, “maintenance” refers to a subject having or keeping the same level or about the same amount of a biomarker (p-tau, Aβ, etc.) in a subject's sample (CSF, blood, etc.) between two time points (one before the administration of lemborexant and the other after administration of lemborexant).

As used herein, “MMSE” refers to the Mini-Mental State Examination, a cognitive instrument commonly used for screening purposes, but also often measured longitudinally in AD clinical trials having a 30 point scale with higher scores indicating less impairment and lower scores indicating more impairment. As used herein, seven items measuring orientation to time and place, registration, recall, attention, language and drawing were assessed. (Folstein, M. F. et al., “Mini-mental state. A practical method for grading the cognitive state of subjects for the clinician.” J. Psychiatr. Res. 1975; 12:189-98.)

As used herein, “PSQI” refers to the Pittsburgh Sleep Quality Index, a self-rated questionnaire which assesses sleep quality and disturbances over a 1-month time interval. Nineteen individual items generate seven “component” scores: subjective sleep quality, sleep latency, sleep duration, habitual sleep efficiency, sleep disturbances, use of sleeping medication, and daytime dysfunction. The sum of scores for these seven components yields one global score, ranging from 0 to 21, where lower scores denote a healthier sleep quality. Clinical and clinometric properties of the PSQI were assessed over an 18-month period with “good” sleepers (healthy subjects, n=52) and “poor” sleepers (depressed patients, n=54; sleep-disorder patients, n=62). (Buysse D. J. et al., “The Pittsburgh Sleep Quality Index: a new instrument for psychiatric practice and research.” Psychiatry Res. 1989; 28(2):193-213.)

As used herein, “STOP-Bang” refers to the snoring, tiredness, observed apnea, high BP, BMI, age, neck circumference, and male gender (STOP-Bang) questionnaire. The questionnaire consists of eight dichotomous (yes/no) items related to the clinical features of sleep apnea. The total score ranges from 0 to 8. Patients can be classified for obstructive sleep apnea (OSA) risk based on their respective scores. The sensitivity of STOP-Bang score ≥3 to detect moderate to severe OSA (apnea-hypopnea index [AHI]>15) and severe OSA (AHI >30) is 93% and 100%, respectively. (Chung F. et al., “STOP-Bang Questionnaire: A Practical Approach to Screen for Obstructive Sleep Apnea” Chest. 2016, 149(3):631-638).

As used herein, “PSG” refers to polysomnography, a standard diagnostic test for OSA. PSG provides an assessment of OSA as the frequency of apneas and hypopneas per hour of sleep (the apnea-hypopnea index or AHI). The severity of OSA is classified as follows: (a) none/minimal: AHI <5 per hour; (b) mild: AHI ≥5, but <15 per hour; (c) moderate: AHI ≥15, but <30 per hour; and (d) severe: AHI ≥30 per hour. (Alshaer H et al. “Reproducibility and predictors of the apnea hypopnea index across multiple nights” Sleep Sci. 2018, 11(1):28-33).

Subjects with “preclinical AD” or “pre-AD” as described herein, are cognitively normal (e.g., unimpaired) individuals with intermediate or elevated levels of amyloid in the brain. At least two clinical pre-AD states in which subjects have unimpaired cognition have been defined by IWG criteria: pre-symptomatic AD and asymptomatic AD (or “asymptomatic at risk”). Pre-symptomatic AD refers to subjects with an autosomal dominant monogenic mutation for AD, e.g., a mutation in Amyloid precursor protein (APP), Presenilin 1 (PSEN1), or Presenilin 2 (PSEN2), who are cognitively unimpaired. Subjects who are pre-symptomatic but carry the he autosomal dominant monogenic mutation will most likely develop AD. Asymptomatic refers to subjects who do not have clinical signs and symptoms of AD, but have the presence of one or more biomarkers of AD pathology. Stages of asymptomatic at risk may be further classified. Episodic memory and executive function deficits may emerge later. Accordingly, subjects with pre-AD may be identified by cognitively unimpaired asymptomatic stages. Cognitively normal can include individuals who are CDR 0, or individuals within the normal ranges of cognitive test scores (MMSE, International Shopping List Task, Logical Memory, etc.). Preclinical AD occurs prior to significant irreversible neurodegeneration and cognitive impairment and is typically characterized by the appearance of in vivo molecular biomarkers of AD and the absence clinical symptoms. Preclinical AD biomarkers that may suggest the future development of Alzheimer's disease include, but are not limited to, intermediate or elevated levels of amyloid in the brain as measured by amyloid or tau positron emission tomography (PET) (e.g., a centiloid measure of about 20-40, e.g., a measure of about 20-32). Additional biomarkers may be used alone or in conjunction, including one or more of cerebrospinal fluid level of Aβ1-42 and/or Aβ 1-42/1-40 ratio, cerebrospinal fluid level of total tau, cerebrospinal fluid level of neurogranin, cerebrospinal fluid level of neurofilament light peptide (NfL), and biomarkers measured in the serum or plasma (e.g. levels of Aβ1-42, the ratio of two forms of amyloid-b peptide (e.g., a ratio of Aβ1-42/1-40 ratio of between about 0.092-0.094 or below about 0.092), plasma levels of plasma total tau (T-tau), levels of phosphorylated tau (P-tau) isoforms (including tau phosphorylated at 181 (P-tau181), 217 (P-tau217), and 231 (P-tau231)), glial fibrillary acidic protein (GFAP), and neurofilament light (NfL)). In addition, certain risk factors contribute to development of AD. For example, subjects who carry the E4 allele of apolipoprotein E (APOE) are also at greater risk for developing AD and subjects with trisomy 21 that includes the APP gene are at greater risk for cerebral amyloidosis. Other risk factors associated with AD include, but are not limited to, having a family history with a first degree relative having AD or dementia, having an age of 65 years or older, being female, having or recovering from traumatic brain injury, suffering from other conditions such as obesity, diabetes, heart and/or blood vessel disease, cancer, and/or immune system dysfunction, and/or a sleep disorder such as insomnia or a circadian rhythm sleep disorder, or having lifestyle risk factors such as smoking, alcohol consumption, lack of exercise, lack of cognitive activity, and malnutrition, and having exposure to environmental risk factors such as air pollution, metals (e.g., aluminum, copper, and zinc).

“Early AD” or “early Alzheimer's disease,” (EAD) as used herein, is a continuum of AD severity from mild cognitive impairment due to AD—intermediate likelihood to mild Alzheimer's disease dementia. Subjects with early AD include subjects with mild Alzheimer's disease dementia as defined herein and subjects with mild cognitive impairment (MCI) due to AD—intermediate likelihood as defined herein. In some embodiments, subjects with early AD have MMSE scores of 22 to 30 and Clinical Dementia Rating (CDR) global range 0.5 to 1.0.

Other methods for detecting early AD disease may employ the tests and assays specified below, including the National Institute of Aging-Alzheimer's Association (NIA-AA) core clinical criteria for probable Alzheimer's disease dementia in McKhann, G. M. et al, “The diagnosis of dementia due to Alzheimer's disease: Recommendations from the National Institute on Aging-Alzheimer's Association workgroups on diagnostic guidelines for Alzheimer's disease.” Alzheimer Dement. 2011; 7:263-9. Other methods include CDR-SB, ADCOMS Composite Clinical Score, the Mini-Mental State Examination, ADAS-Cog, ADAS MCI-ADL, modified iADRS, Wechsler Memory Scale-IV Logical Memory (subscale) I (WMS-IV LMI), and Wechsler Memory Scale-IV Logical Memory (subscale) II (WMS-IV LMII). In some embodiments, a subject with early AD has evidence of elevated amyloid in the brain or a positive amyloid load. In some embodiments, elevated amyloid in the brain or a positive amyloid load is indicated and/or confirmed by PET assessment. In some embodiments, elevated amyloid in the brain or a positive amyloid load is indicated and/or confirmed by a CSF assessment of markers such as Aβ1-42 (e.g., a soluble CSF biomarker analysis). For example, subjects with AD may be selected according to the methods in WO 2023/283650, the contents of which are incorporated herein by reference. In some embodiments, diagnostic thresholds may be identified by amyloid PET either by visual read (per the label of approved PET tracers) or by establishing a centiloid threshold above which subjects are considered to have elevated amyloid (e.g., varying between 15-40 centiloids).

In some embodiments, elevated amyloid in the brain or a positive amyloid load is indicated and/or confirmed by measuring the concentration of Aβ42 and a concentration of Aβ40 and calculating a ratio of Aβ42 to Aβ40 (Aβ42/40 ratio). In some embodiments, elevated amyloid in the brain or a positive amyloid load is indicated and/or confirmed by an MRI or PET. In some embodiments, elevated amyloid in the brain or a positive amyloid load is indicated by retinal amyloid accumulation. In some embodiments, more than one assessment method is used. Subjects with “mild Alzheimer's disease dementia,” as used herein, are subjects meet the NIA-AA core clinical criteria for probable Alzheimer's disease dementia in McKhann, G. M. et al., “The diagnosis of dementia due to Alzheimer's disease: Recommendations from the National Institute on Aging-Alzheimer's Association workgroups on diagnostic guidelines for Alzheimer's disease.” Alzheimer Dement. 2011; 7:263-9. Also included herein are subjects who have a CDR score of 0.5 to 1.0 and a Memory Box score of 0.5 or greater at screening and baseline.

In some embodiments, the subject has “elevated amyloid” or “intermediate amyloid.” As one of ordinary skill in the art will recognize, amyloid levels from amyloid PET can be reported using the Centiloid method in “centiloid” units (CL). (Klunk W E et al. The Centiloid Project: standardizing quantitative amyloid plaque estimation by PET. Alzheimer's Dement. 2015; 11:1-15 el-4). The Centiloid method measures a tracer on a scale of 0 CL to 100 CL, where 0 is deemed the anchor-point and represents the mean in young healthy controls and 100 CL represents the mean amyloid burden present in subjects with mild to moderate severity dementia due to AD. (Id.) As is known to one of ordinary skill in the art, centiloid thresholds may vary, for example may be refined, based on new or additional scientific information. (See, e.g., http://www.gaain.org/centiloid-project.) An elevated level of amyloid can be set relative to a baseline threshold in a healthy control determined according to methods known to a POSA. For example, a centiloid value of 32.5 can be used as a threshold value for “elevated amyloid,” and an “intermediate amyloid” level refers to an Aβ amyloid PET in the range of 20-32.5 CL (e.g., 30 CL). In another example, a centiloid value of 40 can be used as a threshold value for “elevated amyloid,” and an “intermediate amyloid” level refers to an Aβ amyloid PET in the range of 20-40 CL.

Subjects with “MCI due to AD—intermediate likelihood,” as used herein are those identified as such in accordance with the NIA-AA core clinical criteria for mild cognitive impairment due to Alzheimer's disease-intermediate likelihood. For example, symptomatic but not demented AD subjects with evidence of brain amyloid pathology making them less heterogeneous and more similar to mild Alzheimer's disease dementia subjects in cognitive and functional decline as measured by the ADCOMS Composite Clinical Score defined herein. Also included are subjects who have a CDR score of 0.5 and a Memory Box score of 0.5 or greater at screening and baseline. Furthermore, subjects who report a history of subjective memory decline with gradual onset and slow progression over the last 1 year before screening, which is corroborated by an informant, are also included herein.

As used herein, “ADAS-cog” refers to Alzheimer's Disease Assessment Scale-Cognitive. The ADAS-cog is a widely used cognitive scale in Alzheimer's disease trials having a structured scale that evaluates memory (word recall, delayed word recall, and word recognition), reasoning (following commands), language (naming, comprehension), orientation, ideational praxis (placing letter in envelope) and constructional praxis (copying geometric designs). (Rosen, W. G. et al., “A new rating scale for Alzheimer's disease.” Am. J. Psychiatry 1984; 141:1356-64.) Ratings of spoken language, language comprehension, word finding difficulty, ability to remember test instructions, maze, and number cancellation were also obtained. The modified version used herein is scored from 0 to 90 points with a score of 0 indicating no impairment, and a score of 90 indicating maximum impairment.

As used herein, “CDR-SB” refers to clinical dementia rating-sum of boxes. The CDR is a clinical scale that describes 5 degrees of impairment in performance on each of 6 categories of function including memory, orientation, judgment and problem solving, community affairs, home and hobbies, and personal care. (Berg, L. et al., “Mild senile dementia of the Alzheimer type: 2. Longitudinal assessment.” Ann. Neurol. 1988; 23:477-84.) The ratings of degree of impairment obtained on each of the 6 categories of function are synthesized into 1 global rating of dementia CDR score (ranging from 0 to 3). A sum of boxes score provides an additional measure of change where each category has a maximum possible score of 3 points and the total score is a sum of the category scores giving a total possible score of 0 to 18 with higher scores indicating more impairment. The global score may be used as a clinical measure of severity of dementia.

As used herein, “ADCOMS” refers to Alzheimer's Disease Composite Score, a composite clinical score based on an analysis of four ADAS-Cog items (delayed word recall, orientation, word recognition, and word finding difficulty), two MMSE items (orientation to time, and drawing), and all six CDR-SB items (personal care, community affairs, home and hobbies, memory, orientation, and judgment and problem solving), as discussed in the Examples and in Wang, J. et al., “ADCOMS: a composite clinical outcome for prodromal Alzheimer's disease trials.” J. Neurol, Neurosurg. Psychiatry. 2016; 87:993-999. ADCOMS was developed to be particularly sensitive to disease progression during early stages of AD, i.e., prodromal and mild AD.

As used herein, “ApoE4-positive” subjects and “ApoE4 carriers” refer to subjects who harbor the ε4 variant of the apolipoprotein gene. The ε4 variant is one of several major alleles of the apolipoprotein gene. The gene is generally responsible for metabolism of fats. It has been found that carriers of the apolipoprotein ε4 show significantly greater rates of amyloid retention when compared to non-carriers. (Drzezga, A. et al, “Effect of APOE genotype on amyloid plaque load and gray matter volume in Alzheimer disease.” Neurology. 2009; 72:1487-94.) In some embodiments, the subject is a heterozygous carrier of the apolipoprotein E ε4 gene allele. In some embodiments, the subject is a homozygous carrier of the apolipoprotein E ε4 gene allele.

As used herein, the term “clinical decline” refers to a worsening of one or more clinical symptoms of AD. Methods for measuring clinical decline may employ the tests and assays specified herein. In some embodiments, clinical decline is determined by a worsening of ADCOMS. In some embodiments, clinical decline is determined by a worsening of MMSE. In some embodiments, clinical decline is determined by a worsening of ADAS-Cog. In some embodiments, clinical decline is determined by a worsening of FAQ. In some embodiments, clinical decline is determined by a worsening of CDR-SB. In some embodiments, clinical decline is determined by a worsening of Wechsler Memory Scale-IV Logical Memory (subscale) I and/or (subscale) II. In some embodiments, clinical decline is determined by a worsening of CDR score. In some embodiments, clinical decline refers to a worsening in one or more biomarkers of AD or brain measurement (e.g., by PET or MRI), e.g., of brain atrophy and/or amyloid accumulation.

As used herein, the term “treat” (also “treating” or “treatment”) refers to any administration or application of a therapeutic agent to a subject having a disease or disorder, and includes inhibiting the disease, slowing progression of the disease, delaying progression, arresting its development, reversing progression of disease (e.g., reversing build up of Aβ fibrils), preventing the onset of the disease or at least one symptom of the disease, or preventing further development of the disease, relieving or ameliorating one or more symptoms or underlying condition(s) of the disease, curing the disease, improving one or more clinical metrics, or preventing reoccurrence of one or more symptoms of the disease. In some embodiments, treating may comprise maintaining (i.e., preventing from worsening in) the severity of at least one symptom of the disease, for example, when the symptom would otherwise be expected to progress and/or worsen in the absence of administration or application of a therapeutic agent to a subject. In some embodiments, maintaining the symptom may refer to the absence of a change (e.g., no significant change such as no statistically significant change) in the symptom after administration or application of the therapeutic agent to the subject, as compared to a control (e.g., a subject not receiving a therapy or receiving a placebo), for whom a change occurs (e.g., a significant change such as a statistically significant change) in the symptom. Complete treatment is not required. In some embodiments, treatment of AD in a subject comprises an administration, e.g., an intravenous infusion, of a dual orexin receptor antagonist, e.g., lemborexant, e.g., to a subject at risk of developing AD but who does not yet show evidence of dementia.

As used herein, the term “prevent,” which is encompassed within the term “treat” unless context indicates otherwise, refers to obtaining beneficial or desired prophylactic benefit. For prophylactic benefit, the composition may be administered to a subject at risk of developing Alzheimer's disease (e.g., based on a biomarker and/or family history), to a subject having one or more preclinical symptoms but not clinical symptoms of Alzheimer's disease, and/or to a subject reporting one or more of the physiological symptoms of Alzheimer's disease, even though a clinical diagnosis of having Alzheimer's has not been made. As used herein “prevention” may further include therapeutic benefit, by which is meant eradication or amelioration of the underlying condition being treated or of one or more of the physiological symptoms associated therewith. Prevention also encompasses arresting or slowing the further progression of one or more symptoms of a disease.

As used herein, a “control” (also, “control sample”) refers to a biological sample obtained from a subject that is distinct from those samples being evaluated and has a known AD status. In some embodiments, the control samples are obtained from a subject who does not have a diagnosis of Alzheimer's disease, e.g., according to one or more of the definitions above. For example, the control samples may be obtained from a subject who does not have clinical symptoms of AD (e.g., cognitive impairment and/or dementia), and/or does not have any of the markers of AD pathology (e.g., PET scan or CSF analysis for biomarkers such as amyloid or tau). In some embodiments, a control sample may be obtained from a healthy subject. In some embodiments, a control may be obtained from a subject with co-morbidities that are not associated with AD. In some embodiments, the control may be obtained from a subject with a diagnosis along the spectrum of AD disease including, for example, preclinical AD or mild cognitive impairment. In some embodiments, a control sample may be a baseline sample collected from a subject prior to initiation of any treatment. In some embodiments, a control sample may be a sample collected from a control subject administered a placebo.

As used herein, the term a “therapeutically effective amount” refers to an amount of a compound or pharmaceutical composition sufficient to product a desired therapeutic effect, e.g., to reverse, arrest, delay, or slow a cognitive decline, and/or to reverse, arrest, delay, or slow the rate of change in one or more biomarkers of AD. One of ordinary skill in the art will understand that the therapeutically effective amount of lemborexant administered to a subject may depend upon a number of factors including pharmacodynamic characteristics, route of administration, frequency of treatment, and health, age, and weight of the subject to be treated and, with the information disclosed herein, will be able to determine the appropriate amount for each subject.

### II. Methods

Disclosed herein is a method of reducing the amount of p-tau in a subject comprising administering to a subject in need thereof a therapeutically effective amount of lemborexant. Also disclosed herein is a method of reducing neurodegeneration in a subject comprising administering to a subject in need thereof a therapeutically effective amount of lemborexant. Also disclosed herein is a method of reducing amyloid beta in a subject comprising administering to a subject in need thereof a therapeutically effective amount of lemborexant.

In one aspect of the present disclosure, lemborexant affects at least one marker of AD pathology. Without being bound by theory, in some embodiments, lemborexant's surprising effects on AD and AD pathology may be related to lemborexant's role in regulating sleep and treating insomnia, among other potential effects on AD pathology. Notably, lemborexant provides benefits not seen by other sleep agents such as doxepin, another drug approved for treating insomnia. For instance, as discussed in the Examples, when each was administered to animal models of AD, lemborexant and doxepin showed differential effects on Aβ plaque development, activation of phagocytic microglia, and expression of biomarkers involved in membrane receptor trafficking and inflammatory activity. In one animal model, lemborexant reduced total amyloid plaque burden (including both diffuse and fibrillar plaques), whereas doxepin reduced only total amyloid burden and not fibrillar plaque burden (Example 3, Section B). Lemborexant also increased activation of phagocytic microglia surrounding Aβ plaques, whereas doxepin did not (Example 3, Section E). Finally, lemborexant significantly upregulated expression of Ifnb1, an inflammatory mediator IFN-beta, the lysosomal protein Rab5a, and the Aβ degrading enzyme Mnp2, whereas doxepin did not (Example 3, section F). Without being bound by theory, these data may indicate that lemborexant and doxepin act through different mechanisms, and suggest that the effects of a drug on sleep may, in some conditions, differ from its effects on AD pathology.

In various embodiments, disclosed herein is a method of reducing or maintaining the amount of p-tau and/or t-tau in a subject, the method comprising administering to a subject in need thereof a therapeutically effective amount of lemborexant. Also disclosed are methods of reducing or maintaining the amount of p-tau in a subject, of increasing dephosphorylation of tau in a subject, of reducing the ratio of p-tau to tau, and/or of reducing the rate of tau phosphorylation, the methods comprising administering to a subject in need thereof a therapeutically effective amount of lemborexant. In some embodiments, the amount of p-tau and/or t-tau in the subject's CSF is decreased or maintained after administration of a therapeutically effective amount of lemborexant as compared to the amount of p-tau and/or t-tau in the subject's CSF prior to such administration. In some embodiments, the amount of p-tau and/or t-tau in the subject's CSF is decreased or maintained after administration of a therapeutically effective amount of lemborexant as compared to the amount of p-tau and/or t-tau in a subject's CSF after administration of a placebo.

Also disclosed herein is a method of reducing neurodegeneration in a subject comprising administering to a subject in need thereof a therapeutically effective amount of lemborexant. Also disclosed herein is a method of reducing or maintaining amyloid beta in a subject comprising administering to a subject in need thereof a therapeutically effective amount of lemborexant.

In some embodiments, the subject in need of the inventive method(s) has demonstrated evidence of at least one disease chosen from Alzheimer's disease, pre-Alzheimer's disease, early Alzheimer's disease, mild cognitive impairment, cerebral amyloid angiopathy, frontotemporal dementia, dementia with Lewy bodies, Lewy body dementia, Parkinson's disease, vascular dementia, limbic-predominant age-related TDP-43 encephalopathy, frontotemporal lobar degeneration, corticobasal degeneration, Pick's disease, multiple system atrophy, and progressive supranuclear palsy.

### III. Subjects in Need of Treatment

In some embodiments, the subject in need of one or more of the disclosed methods has demonstrated evidence of at least one disease chosen from Alzheimer's disease, pre-Alzheimer's disease, early Alzheimer's disease, mild cognitive impairment, cerebral amyloid angiopathy, frontotemporal dementia, dementia with Lewy bodies, Lewy body dementia, Parkinson's disease, vascular dementia, limbic-predominant age-related TDP-43 encephalopathy, frontotemporal lobar degeneration, corticobasal degeneration, Pick's disease, multiple system atrophy, and progressive supranuclear palsy. In some embodiments, the subject in need thereof has demonstrated evidence of at least one disease chosen from Alzheimer's disease, pre-Alzheimer's disease, and early Alzheimer's disease. In some embodiments, the subject in need thereof has demonstrated evidence of mild cognitive impairment. In some embodiments, the subject in need thereof has demonstrated evidence of cerebral amyloid angiopathy, frontotemporal dementia, dementia with Lewy bodies, Lewy body dementia, vascular dementia, limbic-predominant age-related TDP-43 encephalopathy, frontotemporal lobar degeneration, corticobasal degeneration. In some embodiments, the subject in need thereof has demonstrated evidence of at least one disease chosen from Parkinson's disease, Pick's disease, multiple system atrophy, and progressive supranuclear palsy.

One aspect of the present disclosure relates to a method for treating Alzheimer's disease (AD) in a subject who has AD or is at risk for developing AD, comprising administering to the subject a therapeutically effective amount of lemborexant, a pharmaceutically acceptable salt thereof, or a solvate thereof, thereby treating AD. In some embodiments, the subject is in need of treatment (e.g. has AD, pre-AD, or is otherwise at risk of developing AD).

In some embodiments, treating AD refers one or more of inhibiting, slowing progression of, slowing a rate of progression of, delaying progression of, arresting development of, and reversing progression of AD, AD pathology, symptoms of AD, and/or underlying conditions of AD. In some embodiments, treating refers to preventing the onset of or preventing development of AD, AD pathology, symptoms of AD, and/or underlying conditions of AD. In some embodiments, treating refers to relieving or ameliorating one or more symptoms or underlying condition(s) of AD (e.g., ameliorating build-up of Aβ fibrils) and/or improving one or more clinical metrics of AD (e.g., cognitive function, brain amyloid or tau levels, and/or expression of a biomarker). In some embodiments, treating refers to preventing occurrence or reoccurrence of one or more symptoms of AD.

In some embodiments, treating AD comprises reducing, halting, or slowing cognitive decline.

In some embodiments, a subject in need of treatment described herein is a subject who has AD, e.g., who has been diagnosed with AD. The diagnosis may be based on cognitive evaluation. The diagnosis may be based on measurements of AD pathology obtained by brain imaging (e.g., amyloid PET or tau PET) and/or expression of biomarkers in the subject. In some embodiments, biomarkers comprise brain amyloid level, brain tau level, cerebrospinal fluid level of Aβ1-42, cerebrospinal fluid level of total tau, cerebrospinal fluid level of neurogranin, and cerebrospinal fluid level of neurofilament light chain (NfL). In some embodiments, the subject who has AD shows cognitive impairment and AD pathology. For example, a subject who has AD may have t-tau levels above 400 ng/L, Aβ1-42 levels below 550 ng/L, and/or a Aβ1-42/Aβ1-40 ratio below 0.065.

In some embodiments, the subject in need of treatment has early Alzheimer's disease (also called “Early AD” or “EAD”). In some embodiments, a subject with early AD has symptoms on a continuum of AD severity ranging from mild cognitive impairment due to AD—intermediate likelihood to mild Alzheimer's disease dementia. In some embodiments, a subject with early AD has mild Alzheimer's disease dementia as defined herein and/or mild cognitive impairment (MCI) due to AD—intermediate likelihood as defined herein. In some embodiments, a subject has an MMSE score of 22 to 30 and Clinical Dementia Rating (CDR) global range 0.5 to 1.0. In some embodiments, a subject with early AD has evidence of elevated amyloid in the brain or a positive amyloid load. In some embodiments, elevated amyloid in the brain or a positive amyloid load is indicated and/or confirmed by PET assessment. In some embodiments, elevated amyloid in the brain or a positive amyloid load is indicated and/or confirmed by a CSF assessment of markers such as Aβ1-42 (e.g., a soluble CSF biomarker analysis). In some embodiments, elevated amyloid in the brain or a positive amyloid load is indicated and/or confirmed by measuring a ratio of Aβ42 to Aβ40 (Aβ42/40 ratio). In some embodiments, elevated amyloid in the brain or a positive amyloid load is indicated and/or confirmed by an MRI. In some embodiments, elevated amyloid in the brain or a positive amyloid load is indicated by retinal amyloid accumulation. In some embodiments, more than one assessment method is used.

In some embodiments, the subject has pre-clinical Alzheimer's disease (also called “pre-AD”). A subject with pre-AD may be cognitively normal, with intermediate or elevated levels of amyloid in the brain. In some embodiments, a subject who has pre-AD may be identified by asymptomatic stages with or without memory complaints and emerging episodic memory and executive function deficits. In some embodiments, the subject who has pre-AD has a CDR 0 and/or scores within the normal ranges of cognitive test scores (e.g., MMSE, International Shopping List Task, Logical Memory, etc.). In some embodiments, the subject has other biomarkers that suggest the future development of AD, such as one or more of intermediate or elevated levels of amyloid in the brain by amyloid or tau positron emission tomography (PET) (e.g., a centiloid measure of about 20-40, e.g., a measure of about 20-32), cerebrospinal fluid level of Aβ1-42 and/or Aβ 1-42/1-40 ratio, cerebrospinal fluid level of total tau, cerebrospinal fluid level of neurogranin, cerebrospinal fluid level of neurofilament light peptide (NfL), and blood biomarkers as measured in the serum or plasma (e.g. levels of Aβ1-42, the ratio of two forms of amyloid-b peptide (Aβ1-42/1-40 ratio, e.g., a ratio of between about 0.092-0.094 or below about 0.092), plasma levels of plasma total tau (T-tau), levels of phosphorylated tau (P-tau) isoforms (including tau phosphorylated at 181 (P-tau181), 217 (P-tau217), and 231 (P-tau231)), glial fibrillary acidic protein (GFAP), and neurofilament light (NfL)). In some embodiments, the subject who has pre-AD may have intermediate amyloid (e.g., approximately 20-40 centiloids). In some embodiments, the subject who has pre-AD may have elevate amyloid (e.g., >40 centiloids).

In some embodiments, the subject may be at risk of developing AD. A subject may have one or more risk factors for developing AD, such as carrying a familial AD gene (e.g., the apolipoprotein E ε4 allele, also called “APOE4” or “ApoE4”), having a family history with a first degree relative having AD or dementia, having an age of 65 years or older, being female, having or recovering from traumatic brain injury, suffering from other conditions such as obesity, diabetes, heart and/or blood vessel disease, and more. In some embodiments, a subject who has pre-AD is at risk of developing AD. The risk of developing AD may be a greater risk than a control subject for developing AD at all and/or for a greater risk for developing AD sooner than the time estimated for a control subject. For example, a subject who has pre-AD and who is cognitively normal but has intermediate amyloid PET levels (approximately 20-40 centiloids) may be at risk for further Aβ accumulation and early spread of tau pathology over 4 years, as compared to a control subject. A subject who has pre-AD and who is cognitively normal but has elevated amyloid PET levels (>40 centiloids) may be at high risk for cognitive decline over 4 years, as compared to a control subject.

In some embodiments, treating AD comprises affecting a change (for example, slowing, delaying, or reducing) in at least one marker of AD pathology.

In some embodiments, the marker of AD pathology is a level of phosphorylation of tau, neurodegeneration, a change in microglial response, and/or presence of Aβ plaques. The marker of AD pathology may be present in a brain region in the subject, such as the hippocampus, somatomotor cortex, somatosensory cortex, piriform cortex, and/or entorhinal cortex. In some embodiments, the marker of AD pathology is detected in a brain scan. For example, tau phosphorylation may be detected by tau PET; Aβ may be detected by amyloid PET. In some embodiments, the marker of AD pathology is detected in a body fluid of the subject, such as blood (e.g., plasma) or CSF. For example, various species of phosphorylated tau and Aβ may be detected in plasma or CSF from a subject.

In some embodiments, the subject does not show signs of dementia and/or cognitive impairment. In some embodiments, the subject has mild cognitive impairment or mild dementia.

In some embodiments, the subject is amyloid positive. The subject may be at risk for further Aβ accumulation and/or spread of tau pathology. The subject may be at risk for cognitive decline. In some embodiments, the subject may have intermediate levels of amyloid PET (e.g., approximately 20-40 centiloids). In some embodiments, the subject may have elevated levels of amyloid PET (e.g., >40 centiloids). In some embodiments, the subject may carry the APOE4 gene. In some embodiments, the subject may have one or more risk factors for developing AD, such as having a family history with a first degree relative having AD or dementia, having an age of 65 years or older, being female, having or recovering from traumatic brain injury, and suffering from other conditions such as obesity, diabetes, heart disease, and/or blood vessel disease.

In some embodiments, whether the subject has been diagnosed with AD, based on brain imaging, cognitive function, and/or biomarker criteria. In some embodiments, the subject has early AD. In some embodiments, the subject has pre-AD.

In some embodiments, treatment may slow cognitive decline and/or reduce a rate of change in a biomarker of AD.

### IV. Tau and Aβ

The ratio of the concentration of p-tau (also referred to herein as “phospho-tau” or “phosphorylated tau”) to the concentration of t-tau (also referred to herein as “total tau”) in the CSF of a subject (herein “ratio of CSF p-tau/t-tau”) can be used to assess the amount of phosphorylation of tau. The concentration of p-tau and of t-tau in the CSF of a subject is measured using liquid chromatography with tandem mass spectroscopy (LC MS/MS).

In some embodiments, the ratio of CSF p-tau/t-tau in a subject to whom a therapeutically effective amount of lemborexant was administered is reduced compared to the ratio of CSF p-tau/t-tau of a subject to whom placebo was administered. In some embodiments, the ratio of CSF p-tau/t-tau in a subject to whom a therapeutically effective amount of lemborexant was administered is maintained within (i.e., +/−) 10% of the ratio of CSF p-tau/t-tau of a subject to whom placebo was administered. In some embodiments, the ratio of CSF p-tau/t-tau in a subject to whom a therapeutically effective amount of lemborexant was administered is within 9%, within 8%, within 7%, within 6%, within 5%, within 4%, within 3%, within 2%, or within 1% of the ratio of CSF p-tau/t-tau of a subject to whom placebo was administered.

In some embodiments, the ratio of CSF p-tau/t-tau in a subject to whom a therapeutically effective amount of lemborexant was administered is reduced compared to the ratio of CSF p-tau/t-tau of the subject prior to the administration of lemborexant.

In some embodiments, the ratio of CSF p-tau/t-tau in a subject to whom a therapeutically effective amount of lemborexant was administered is maintained within (i.e., +/−) 10% of the ratio of CSF p-tau/t-tau of the subject prior to the administration of lemborexant. In some embodiments, the ratio of CSF p-tau/t-tau in a subject to whom a therapeutically effective amount of lemborexant was administered is within 9%, within 8%, within 7%, within 6%, within 5%, within 4%, within 3%, within 2%, or within 1% of the ratio of CSF p-tau/t-tau of the subject prior to the administration of lemborexant.

In some embodiments, the concentration of amyloid beta (Aβ) in the CSF is lower than the concentration of Aβ in the CSF of a subject to whom placebo was administered. In some embodiments, the concentration of Aβ in the CSF is lower than the concentration of Aβ in the CSF of a subject prior to the administration of lemborexant. In some embodiments, the concentration of Aβ38, Aβ40, and/or Aβ42 in the CSF is/are reduced. In some embodiments, the concentration of Aβ in the CSF is maintained in comparison to the concentration of Aβ in the CSF of a subject prior to the administration of lemborexant. In some embodiments, the concentration of Aβ38, Aβ40, and/or Aβ42 in the CSF is/are maintained in comparison to the concentration of Aβ in the CSF of a subject prior to the administration of lemborexant. In some embodiments, the amyloid PET signal in the brain of the subject to whom a therapeutically effective amount of lemborexant is administered is lower than the amyloid PET signal in the brain of a subject to whom placebo is administered. In some embodiments, the amyloid PET signal in the brain of the subject to whom a therapeutically effective amount of lemborexant is administered is lower or maintained than the amyloid PET signal in the brain of a subject prior to administration of lemborexant. In some embodiments, the increase in the concentration of Aβ in the CSF of a subject to whom lemborexant was administered is smaller than the increase in concentration of Aβ in the CSF of a subject to whom placebo was administered.

In some embodiments, the concentration of Aβ in the CSF of a subject to whom lemborexant was administered is at least 5% lower compared to a subject to whom placebo was administered. In some embodiments, the concentration of Aβ in the CSF of a subject to whom lemborexant was administered is at least 10%, at least 15%, at least 20%, or at least 25% lower compared to a subject to whom placebo was administered.

In some embodiments, the concentration of Aβ in CSF is measured using liquid chromatography with tandem mass spectroscopy (LC MS/MS). In some embodiments, the concentration of Aβ38, Aβ40, and/or Aβ42 in the CSF is measured using LC MS/MS. Methods for measuring Aβ38, Aβ40, and Aβ42 are known in the art, such as assays using LC MS/MS. Methods may include the PrecivityAD™ assay (see, e.g., Kirmess et al., J. Clinica Chimica Acta 519: 267-275 (2021)) and the Sysmex assay (https://www.eisai.com/news/2019/news201990.html) for measuring Aβ42 and Aβ40 in a blood or plasma sample or a CSF sample. In some embodiments, the concentration of Aβ in the CSF is measured using ELISA. In some embodiments, the concentration of Aβ38, Aβ40, and/or Aβ42 in the CSF is measured using ELISA. Methods for measuring Aβ are known in the art. See Englund, H. et al., J. Neurochem. 103:334-45 (2007). In some embodiments, the reduction or maintenance of concentration of Aβ38, Aβ40, and/or Aβ42 is compared to a subject prior to administration of lemborexant. In some embodiments, administration to a subject of a composition comprising a therapeutically effective amount of lemborexant results in a reduction of at least 1%, at least 2%, at least 3%, at least 4%, at least 5%, at least 6%, at least 7%, at least 8%, at least 9%, at least 10%, at least 11%, at least 12%, or at least 13% relative to baseline, of cerebrospinal fluid level concentration of Aβ38, Aβ40, and/or Aβ42.

In some embodiments, p-tau and t-tau are reduced. In some embodiments, p-tau, t-tau, and/or aggregated tau is reduced. In some embodiments, the reduction of tau phosphorylation occurs in the whole brain or areas of the brain such as, for example, the frontal lobe, parietal lobe, temporal lobe, occipital lobe, cingulate cortex, amygdala, hippocampus, entorhinal cortex, and/or piriform cortex. In some embodiments, the tau PET signal in the brain is decreased compared to placebo. In some embodiments, the tau PET signal in the brain is decreased or maintained compared to baseline.

In some embodiments, administration to a subject of a therapeutically effective amount of lemborexant results in a reduction of concentration of p-tau in the CSF of the subject compared to the concentration of p-tau in the CSF of the subject to whom placebo was administered. In some embodiments, administration to a subject of a therapeutically effective amount of lemborexant results in a reduction of concentration of p-tau in the CSF of the subject compared to the concentration of p-tau in the CSF of the subject prior to the administration of lemborexant. In some embodiments, administration to a subject of a therapeutically effective amount of lemborexant results in the maintenance of the amount of p-tau in the CSF of the subject compared to the concentration of p-tau in the CSF of the subject to whom placebo was administered. In some embodiments, administration to a subject of a therapeutically effective amount of lemborexant results in the maintenance of the amount of p-tau in the CSF of the subject prior to the administration of lemborexant.

In some embodiments, the concentration of p-tau in the CSF is measured using liquid chromatography with tandem mass spectroscopy (LC MS/MS).

In some embodiments, administration to a subject of a therapeutically effective amount of lemborexant results in the maintenance of the amount of p-tau in the CSF of the subject compared to the concentration of p-tau in the CSF of the subject to whom placebo was administered. In some embodiments, administration to a subject of a therapeutically effective amount of lemborexant results in a reduction of at least 1%, at least 2%, at least 3%, at least 4%, at least 5%, at least 6%, at least 7%, at least 8%, at least 9%, at least 10%, at least 11%, at least 12%, or at least 13% of the amount of p-tau in the CSF of the subject compared to the amount of p-tau in the CSF of the subject to whom placebo was administered. In some embodiments, administration to a subject of a therapeutically effective amount of lemborexant results in a reduction of at least 1%, at least 2%, at least 3%, at least 4%, at least 5%, at least 6%, at least 7%, at least 8%, at least 9%, at least 10%, at least 11%, at least 12%, or at least 13% of the amount of p-tau in the CSF of the subject compared to the amount of p-tau in the CSF of the subject prior to the administration of lemborexant.

In some embodiments, the increase in the amount of p-tau in the CSF of a subject to whom lemborexant was administered is lower than the increase in amount of p-tau in the CSF of a subject to whom placebo was administered.

In some embodiments, administration to a subject of a therapeutically effective amount of lemborexant results in a maintenance of or reduction of concentration of p-tau in the CSF of the subject compared to the concentration of p-tau in the CSF of the subject at baseline through 18 months after administration of lemborexant. In some embodiments, administration to a subject of a therapeutically effective amount of lemborexant results in a reduction of at least 1%, at least 2%, at least 3%, at least 4%, at least 5%, at least 6%, at least 7%, at least 8%, at least 9%, at least 10%, at least 11%, at least 12%, or at least 13% concentration of p-tau in the CSF of the subject compared to the concentration of p-tau in the CSF of the subject at baseline.

In some embodiments, the reduction of the amount of p-tau or t-tau in a subject's CSF is due to a reduction or maintenance in the amount of p-tau or t-tau in a subject comprising administering to a subject in need thereof a therapeutically effective amount of lemborexant. In some embodiments, the reduction of the amount of p-tau in a subject's CSF is due to an increase in the dephosphorylation of p-tau. In some embodiments, the reduction of the amount of p-tau in a subject's CSF is due to a reduction of the amount of tau. In some embodiments, the reduction of the amount of p-tau in a subject's CSF is due to a reduction of ratio of p-tau/t-tau.

In some embodiments, administration to a subject of a therapeutically effective amount of lemborexant disclosed results in a reduction or maintenance of the concentration of p-tau in the CSF of the subject compared to the concentration of p-tau in the CSF of the subject to whom placebo was administered. In some embodiments, administration to a subject of a therapeutically effective amount of lemborexant results in a reduction of at least about 5 pg/mL, at least about 10 pg/mL, at least about 15 pg/mL, at least about 20 pg/mL, at least about 25 pg/mL, at least about 30 pg/mL, at least about 35 pg/mL, or at least about 40 pg/mL, relative to placebo, of cerebrospinal fluid amount of p-tau. In some embodiments, administration to a subject of a composition comprising a therapeutically effective amount of lemborexant disclosed herein results in a reduction of at least about 40 μg/mL, relative to placebo, of cerebrospinal fluid amount of p-tau.

In some embodiments, administration to a subject of a therapeutically effective amount of lemborexant disclosed results in a reduction or maintenance of the concentration of p-tau in the CSF of the subject compared to the concentration of p-tau in the CSF of the subject prior to the administration of lemborexant. In some embodiments, administration to a subject of a therapeutically effective amount of lemborexant results in a reduction of at least about 5 pg/mL, at least about 10 pg/mL, at least about 15 pg/mL, at least about 20 pg/mL, at least about 25 pg/mL, at least about 30 pg/mL, at least about 35 pg/mL, or at least about 40 pg/mL, relative to baseline, of cerebrospinal fluid amount of p-tau. In some embodiments, administration to a subject of a composition comprising a therapeutically effective amount of lemborexant disclosed herein results in a reduction of at least about 40 pg/mL, relative to baseline, of cerebrospinal fluid amount of p-tau.

In some embodiments, administration to a subject of a therapeutically effective amount of lemborexant results in a reduction of at least about 5 pg/mL, at least about 10 pg/mL, at least about 15 pg/mL, at least about 20 pg/mL, at least about 25 pg/mL, at least about 30 pg/mL, at least about 35 pg/mL, or at least about 40 pg/mL, relative to baseline, of cerebrospinal fluid amount of p-tau through 18 months after administration of the composition comprising a therapeutically effective amount of lemborexant. In some embodiments, administration to a subject of a composition comprising a therapeutically effective amount of lemborexant results in a reduction of at least 40 pg/mL, relative to baseline, of cerebrospinal fluid amount of p-tau through 18 months after administration of the composition comprising a therapeutically effective amount of at least lemborexant.

In some embodiments, administration to a subject of a therapeutically effective amount of lemborexant results in a reduction of at least about 5 pg/mL, at least about 10 pg/mL, at least about 15 pg/mL, at least about 20 pg/mL, at least about 25 pg/mL, at least about 30 pg/mL, at least about 35 pg/mL, or at least about 40 pg/mL, relative to placebo, of cerebrospinal fluid amount of p-tau through 18 months after administration of the composition comprising a therapeutically effective amount of lemborexant. In some embodiments, administration to a subject of a composition comprising a therapeutically effective amount of lemborexant results in a reduction of at least 40 pg/mL, relative to placebo, of cerebrospinal fluid amount of p-tau through 18 months after administration of the composition comprising a therapeutically effective amount of at least lemborexant.

In some embodiments, the amount of p-tau is reduced within 48 hours of administration of the first dose of lemborexant to the subject relative to the subject's baseline. In some embodiments, the amount of p-tau is reduced within 24 hours of administration of the first dose of lemborexant to the subject relative to the subject's baseline. In some embodiments, the amount of p-tau is reduced within 12 hours of administration of the first dose of lemborexant to the subject relative to the subject's baseline. In some embodiments, the amount of p-tau is reduced within 6 hours of administration of the first dose of lemborexant to the subject relative to the subject's baseline.

In some embodiments, the amount of t-tau is reduced within 48 hours of administration of the first dose of lemborexant to the subject relative to the subject's baseline. In some embodiments, the amount of t-tau is reduced within 24 hours of administration of the first dose of lemborexant to the subject relative to the subject's baseline. In some embodiments, the amount of t-tau is reduced within 12 hours of administration of the first dose of lemborexant to the subject relative to the subject's baseline. In some embodiments, the amount of t-tau is reduced within 6 hours of administration of the first dose of lemborexant to the subject relative to the subject's baseline.

In some embodiments, the amount of Aβ is reduced within 48 hours of administration of the first dose of lemborexant to the subject relative to the subject's baseline. In some embodiments, the amount of Aβ is reduced within 24 hours of administration of the first dose of lemborexant to the subject relative to the subject's baseline. In some embodiments, the amount of Aβ is reduced within 12 hours of administration of the first dose of lemborexant to the subject relative to the subject's baseline. In some embodiments, the amount of Aβ is reduced within 6 hours of administration of the first dose of lemborexant to the subject relative to the subject's baseline.

In some embodiments, the amount of p-tau in a subject's CSF is reduced within 48 hours of administration of the first dose of lemborexant to the subject relative to a subject to whom placebo was administered. In some embodiments, the amount of tau phosphorylation in a subject's CSF is reduced within 24 hours of administration of the first dose of lemborexant to the subject relative to a subject to whom placebo was administered. In some embodiments, the amount of tau phosphorylation in a subject's CSF is reduced within 12 hours of administration of the first dose of lemborexant to the subject relative to a subject to whom placebo was administered. In some embodiments, the amount of tau phosphorylation in a subject's CSF is reduced within 6 hours of administration of the first dose of lemborexant to the subject relative to a subject to whom placebo was administered.

### A. Altering Tau

A further aspect of the present disclosure relates to a method of altering tau (for example, reducing or delaying tau accumulation, tau phosphorylation, and/or tau spreading, and/or slowing a rate thereof) in a subject having AD or at risk for developing AD, comprising administering to the subject a therapeutically effective amount of lemborexant, wherein the therapeutically effective amount is sufficient to reduce alter tau in the subject. In some embodiments, altering tau comprises reducing or delaying tau accumulation, tau phosphorylation, or tau spreading, or slowing a rate of any of these.

In some embodiments, altering tau is slowing progression of, slowing a rate of progression of, delaying progression of, arresting development of, and reversing progression of tau pathology (e.g, tau accumulation, tau phosphorylation, and/or tau spreading), for example, in a brain region. The brain region may be the cortex or the hippocampus. The brain region may be the CA1 region, CA2 region, CA3 region, and/or dentate gyrus of the hippocampus. The brain region may be the entorhinal cortex and/or the piriform cortex. In some embodiments, altering tau is preventing the onset of or preventing development of tau pathology. Altering tau may reduce, delay, or slow a rate of onset and/or progression of symptoms of tau pathology. In some embodiments, altering tau is relieving or ameliorating one or more symptoms of tau pathology and/or improving one or more clinical metrics of tau pathology (e.g., cognitive function, brain amyloid or tau levels, and/or expression of a biomarker). In some embodiments, altering tau is preventing occurrence or reoccurrence of one or more symptoms of tau pathology.

In some embodiments, the subject is amyloid negative. The subject may have mild cognitive impairment or mild dementia. In some embodiments, the subject does not show signs of dementia and/or cognitive impairment.

In some embodiments, the tau is altered relative to a reference. Accordingly, the methods described herein may comprise reducing and/or delaying tau accumulation, tau phosphorylation, and/or tau spreading, and/or slowing the rate of any of these, as compared to a reference. In some embodiments, the reference is a baseline measurement from the subject prior to treatment. In some embodiments, the reference is a baseline measurement from a control subject. The reference may be a measurement obtained from more than one control subjects, which is used as a standard or threshold measurement. In some embodiments, the reference is a measurement from a control subject administered a placebo.

In some embodiments, the methods herein comprise altering (e.g., reducing, arresting, or slowing the growth of) tau in a brain region of the subject. Altering tau in the brain region may comprise altering a tau PET signal in the brain region. In some embodiments, the brain region is the hippocampus, entorhinal cortex, and/or the piriform cortex.

In some embodiments, altering tau comprises altering tau in a body fluid of the subject. For example, tau levels in the subject's brain may be detected in the subject's body fluid. In some embodiments, the body fluid is blood (e.g., plasma) or CSF.

In some embodiments, one or more forms of tau may be altered. In some embodiments, tau is total tau. In some embodiments, tau is aggregated tau. In some embodiments, the tau is a phosphorylated form of tau (phospho-tau). Phospho-tau may be tau that is phosphorylated on one or more of T181, T217, S202, S205, or T231.

In some embodiments, altering tau comprises altering a ratio of phopho-tau to total tau. In some embodiments, the ratio of phospho-tau to total tau is altered such that the ratio is maintained within 10% of the ratio of phospho-tau to total tau of the subject prior to the administration of lemborexant. In some embodiments, a rate of dephosphorylation of phospho-tau is increased. In some embodiments, a rate of phosphorylation of tau is decreased. In some embodiments, altering tau comprises altering tau within 48 hours of administration of a first dose of lemborexant. For example, tau may be reduced within 48 hours of administration of the first dose of lemborexant. In some embodiments, reducing tau comprises altering phospho-tau in the hippocampus, entorhinal cortex, and/or piriform cortex.

### B. Maintaining Tau

A further aspect of the present disclosure relates to a method of maintaining tau (e.g., tau accumulation, tau phosphorylation, and/or tau spreading) in a subject having AD or at risk of developing AD, comprising administering to the subject a therapeutically effective amount of lemborexant, wherein the therapeutically effective amount is sufficient to maintain tau in the subject.

In some embodiments, maintaining tau refers to maintaining tau accumulation, tau phosphorylation, and/or tau spreading, when tau would be expected to progress and/or worsen in the absence of administration or application of a therapeutic agent to a subject. In some embodiments, maintaining tau may refer to the absence of a change (e.g., no significant change such as no statistically significant change) in tau (e.g., tau accumulation, tau phosphorylation, and/or tau spreading) after administration or application of the therapeutic agent to the subject, as compared to a control, for whom administration or application of the therapeutic agent leads to a change (e.g., a significant change such as a statistically significant change) in tau.

In some embodiments, maintaining tau comprises maintaining tau pathology (e.g., tau accumulation, tau phosphorylation, and/or tau spreading), for example, in a brain region. The brain region may be the cortex or the hippocampus. The brain region may be the CA1 region, CA2 region, CA3 region, and/or dentate gyrus of the hippocampus. The brain region may be the entorhinal cortex and/or the piriform cortex. In some embodiments, maintaining tau is preventing the onset of or preventing development of tau pathology, for example, because tau pathology is maintained without change. Maintaining tau may reduce, delay, or slow a rate of onset and/or progression of symptoms of tau pathology, for example, because tau pathology is maintained without change. In some embodiments, maintaining tau may result in relieving or ameliorating one or more symptoms of tau pathology and/or improving one or more clinical metrics of tau pathology (e.g., cognitive function, brain amyloid or tau levels, and/or expression of a biomarker). In some embodiments, maintaining tau may result in preventing occurrence or reoccurrence of one or more symptoms of tau pathology.

In some embodiments, the subject is amyloid negative. The subject may have mild cognitive impairment or mild dementia. In some embodiments, the subject does not show signs of dementia and/or cognitive impairment.

In some embodiments, the subject shows signs of cognitive impairment. In some embodiments, the subject is amyloid positive. In some embodiments, the subject has a diagnosis of AD, e.g., early AD.

In some embodiments, the tau is maintained relative to a reference. Accordingly, the methods described herein may comprise maintaining tau accumulation, tau phosphorylation, and/or tau spreading (or the rate of any of these) as compared to a reference. In some embodiments, the reference is a baseline measurement from the subject prior to treatment. In some embodiments, the reference is a baseline measurement from a control subject. The reference may be a measurement obtained from more than one control subject, and may be used as a standard or threshold measurement. In some embodiments, the reference is a measurement from a control subject administered a placebo.

In some embodiments, maintaining tau comprises maintaining tau in a brain region of the subject. Maintaining tau in the brain region may comprise altering, reducing, or maintaining a tau PET signal in the brain region. In some embodiments, the brain region is the hippocampus, entorhinal cortex, and/or the piriform cortex.

In some embodiments, maintaining tau comprises maintaining tau in a body fluid of the subject. Tau levels in the subject's brain may be correlated with a level in the subject's body fluid. In some embodiments, the body fluid is blood (e.g., plasma) or CSF.

In some embodiments, one or more forms of tau may be maintained. In some embodiments, tau is total tau. In some embodiments, tau is aggregated tau. In some embodiments, the tau is a phosphorylated form of tau (phospho-tau). Phospho-tau may be tau that is phosphorylated on one or more of T181, T217, S202, S205, or T231.

In some embodiments, maintaining tau comprises maintaining a ratio of phopho-tau to total tau. In some embodiments, the ratio of phospho-tau to total tau is maintained within 10% of the ratio of phospho-tau to total tau of the subject prior to the administration of lemborexant. In some embodiments, maintaining tau comprises maintaining tau within 48 hours of administration of a first dose of lemborexant. In some embodiments, phospho-tau is maintained in the hippocampus, entorhinal cortex, and/or piriform cortex.

### V. Microglial Response

In some embodiments, administration to a subject of a therapeutically effective amount of lemborexant results in an increase in the number of activated microglial cells compared to placebo. In some embodiments, the increase in the number of activated microglial cells is measured by PET. In some embodiments, the activated microglial cells are phagocytic microglial cells.

In some embodiments, administration to a subject of a therapeutically effective amount of lemborexant results in an increase in the number of activated microglial cells compared to baseline. In some embodiments, the increase in the number of activated microglial cells is measured by PET. In some embodiments, the activated microglial cells are phagocytic microglial cells.

Methods for measuring microglia are known in the art, such as PET. In some embodiments, the whole brain or at least one area of the brain (for example, cortical gray matter (i.e., cortex), lateral ventricles, frontal lobe, parietal lobe, temporal lobe, occipital lobe, cingulate cortex, amygdala, piriform cortex, entorhinal cortex, hippocampus, hippocampal CA3 (pyramidal neurons), and/or hippocampal dentate gyrus (granule cell neurons) is/are analyzed by PET.

### A. Modulating Microglial Response

One aspect of the present disclosure relates to a method of modulating a microglial response in a subject having Alzheimer's disease (AD) or at risk for developing AD, comprising administering to the subject a therapeutically effective amount of lemborexant, a pharmaceutically acceptable salt thereof, or a solvate thereof, wherein the therapeutically effective amount is sufficient to modulate the microglial response in the subject.

In some embodiments, modulating refers to increasing or decreasing a microglial response, and/or to increasing or decreasing a rate of a microglial response. In some embodiments, the number of microglia do not change, but the response (e.g., activation, reactivation, reactivity, differentiation, etc.) of the microglia is modulated. The modulation may differ depending on brain region (e.g., microglial activation or other response may occur differently in different brain regions).

In some embodiments, modulation of the microglial response is measured in a subject and compared to the microglial response in a reference. In some embodiments, the reference is a baseline measurement from the subject prior to treatment. In some embodiments, the reference is a baseline measurement from a control subject. The reference may be a measurement obtained from more than one control subjects, which is used as a standard or threshold measurement. In some embodiments, the reference is a measurement from a control subject administered a placebo.

In some embodiments, modulating the microglial response comprises modulating expression of at least one microglial marker. The microglial marker may be a general microglial marker. The general microglial marker may be a general marker for microglia in a specific context or disease setting (e.g., a general marker for activated microglia, reactive microglia, and/or microglia in a disease setting). For example, the general microglial marker may be Iba1, Clec7a, or CD68. The microglial marker may be a homeostatic microglial marker. For example, the homeostatic microglial marker is TMEM119 or P2RY12.

In some embodiments, modulating the microglial response comprises modulating activity of phagocytic microglia.

In some embodiments, the subject has mild cognitive impairment or mild dementia. In some embodiments, the subject does not show signs of dementia and/or cognitive impairment.

In some embodiments, the subject is amyloid-negative. The subject may have tau pathology. The subject may have neurodegeneration in a brain region, for example, the hippocampus, the entorhinal cortex, and/or the piriform cortex. In some embodiments, the brain region is the CA1 region, the CA2 region, the CA3 region, or the dentate gyrus in the hippocampus.

In some embodiments, for those subjects who have neurodegeneration, modulating the microglial response comprises modulating a response in microglia associated with degenerating neurons. Microglia associated with neurodegenerating neurons may be close in proximity to the neurons, for example, when observed in a scan or a sample obtained from the subject. In some embodiments, microglia associated with degenerating neurons may phagocytose the degenerating neurons and/or debris therefrom. Accordingly, in some embodiments, administering to these subjects a therapeutically effective amount of lemborexant, a pharmaceutically acceptable salt thereof, or a solvate thereof, may comprise reducing expression of at least one general microglial marker. The general microglial marker may be Iba1, CD68, or Clec7a. In some embodiments, modulating the microglial response comprises increasing expression of at least one homeostatic microglial marker. The homeostatic microglial marker may be TMEM119 or P2RY12.

In some embodiments, the subject is amyloid positive, e.g., the subject has Aβ plaques. The Aβ plaques may be fibrillar Aβ plaques. In some embodiments, the Aβ plaques are present in the hippocampus, the somatomotor cortex, the somatosensory cortex, and/or the piriform cortex of the subject.

In some embodiments, the subject does not show signs of dementia and/or cognitive impairment. In some embodiments, the subject has mild cognitive impairment or mild dementia.

In some embodiments, the subject is at risk for further Aβ accumulation. The subject may be at risk for spread of tau pathology. The subject may be at risk for cognitive decline. In some embodiments, the subject may have intermediate levels of amyloid PET (e.g., approximately 20-40 centiloids). In some embodiments, the subject may have elevated levels of amyloid PET (e.g., >40 centiloids). In some embodiments, the subject may be an ApoE4 carrier. In some embodiments, the subject may have one or more risk factors for developing AD, such as having a family history with a first degree relative having AD or dementia, having an age of 65 years or older, being female, having or recovering from traumatic brain injury, and suffering from other conditions such as obesity, diabetes, heart disease, and/or blood vessel disease.

In some embodiments, the subject has early-stage AD. In some embodiments, the subject has pre-AD.

In some embodiments, in those subjects who are amyloid positive, modulating the microglial response comprises modulating a response in microglia associated with Aβ plaques. Microglia associated with Aβ plaques may be close in proximity to the Aβ plaques, for example, when observed in a scan or a sample obtained from the subject. In some embodiments, microglia associated with Aβ plaques may phagocytose the Aβ plaques. Accordingly, in some embodiments, administering to these subjects a therapeutically effective amount of lemborexant, a pharmaceutically acceptable salt thereof, or a solvate thereof, may comprise increasing expression of a general microglial marker. The general microglial marker may be Iba1, Clec7a, or CD68. In some embodiments, modulating the microglial response comprises increasing phagocytosis of Aβ plaques by phagocytic microglia. In some embodiments, modulating the microglial response comprises reducing expression of a homeostatic microglial marker. The homeostatic microglial marker may be TMEM119 or P2RY12.

### VI. Amyloid Plaques

In some embodiments, administration to a subject of a therapeutically effective amount of lemborexant results in a decrease or maintenance in amyloid plaques compared to placebo. In some embodiments, administration to a subject of a therapeutically effective amount of lemborexant results in a decrease or maintenance in fibrillar amyloid plaques compared to placebo. In some embodiments, administration to a subject of a therapeutically effective amount of lemborexant results in a decrease or maintenance in amyloid plaques compared to baseline. In some embodiments, administration to a subject of a therapeutically effective amount of lemborexant results in a decrease or maintenance in fibrillar amyloid plaques compared to baseline. In some embodiments, the amyloid plaques are fibrillar amyloid plaques.

### A. Altering Aβ Plaques

Another aspect of the present disclosure relates to a method of altering Aβ plaques (e.g., reducing or delaying formation of Aβ plaques, or slowing a rate of growth thereof) in a subject having AD or at risk for developing AD, comprising administering to the subject a therapeutically effective amount of lemborexant, a pharmaceutically acceptable salt thereof, or a solvate thereof, wherein the therapeutically effective amount is sufficient to alter Aβ plaques in the subject.

In some embodiments, altering Aβ plaques comprises slowing progression of, slowing a rate of progression of, delaying progression of, arresting development of, and reversing progression of Aβ plaque formation and/or Aβ plaque growth. In some embodiments, altering Aβ plaques comprises preventing the onset of or preventing development of Aβ plaque pathology (e.g., any pathology resulting from or coinciding with Aβ plaque formation and/or Aβ plaque growth). Altering Aβ plaques may reduce, delay, or slow a rate of onset and/or progression of symptoms of this pathology. In some embodiments, altering Aβ plaques comprises relieving or ameliorating one or more symptoms of Aβ plaque pathology and/or improving one or more clinical metrics of Aβ plaque pathology (e.g., cognitive function, brain amyloid or tau levels, and/or expression of a biomarker). In some embodiments, altering Aβ plaques comprises preventing occurrence or reoccurrence of one or more symptoms of Aβ plaque formation and/or Aβ plaque growth.

In some embodiments, the Aβ plaques are altered relative to a reference. Accordingly, altering Aβ plaques may comprise reducing and/or delaying A plaque formation, and/or slowing the rate thereof, as compared to a reference. In some embodiments, the reference is a baseline measurement from the subject prior to treatment. In some embodiments, the reference is a baseline measurement from a control subject. The reference may be a measurement obtained from more than one control subjects, which is used as a standard or threshold measurement. In some embodiments, the reference is a measurement from a control subject administered a placebo.

In some embodiments, the Aβ plaques are fibrillar plaques. In some embodiments, the Aβ plaques are total plaques, and may comprise non-fibrillar (e.g., diffuse) plaques.

In some embodiments, altering Aβ plaques comprises reducing the growth or rate of growth of Aβ plaques. The reduction in growth of Aβ plaques may be in the hippocampus of the subject, somatomotor cortex, the somatosensory cortex, and/or the piriform cortex of the subject. In some embodiments, altering Aβ plaques comprises altering an amyloid PET signal obtained from a brain region of the subject. In some embodiments, altering Aβ plaques corresponds to a reduction in the concentration of Aβ in the subject's CSF. The Aβ may be Aβ38, Aβ40, and/or Aβ42.

In some embodiments, altering Aβ comprises altering Aβ plaques within 48 hours of administration of a first dose of lemborexant.

In some embodiments, the subject does not show signs of dementia and/or cognitive impairment. In some embodiments, the subject has mild cognitive impairment or mild dementia.

In some embodiments, the subject is at risk for further Aβ accumulation. The subject may also be at risk for spread of tau pathology. The subject may be at risk for cognitive decline. In some embodiments, the subject may have intermediate levels of amyloid PET (e.g., approximately 20-40 centiloids). In some embodiments, the subject may have elevated levels of amyloid PET (e.g., 40 centiloids). In some embodiments, the subject may be an ApoE4 carrier. In some embodiments, the subject may have one or more risk factors for developing AD, such as having a family history with a first degree relative having AD or dementia, having an age of 65 years or older, being female, having or recovering from traumatic brain injury, and suffering from other conditions such as obesity, diabetes, heart disease, and/or blood vessel disease.

In some embodiments, the subject has early-stage AD. In some embodiments, the subject has pre-AD.

### VII. Neurodegeneration

Also disclosed herein is a method of reducing neurodegeneration in a subject comprising administering to a subject in need thereof a therapeutically effective amount of lemborexant. In some embodiments, the subject in need thereof has demonstrated evidence of at least one disease chosen from Alzheimer's disease, pre-Alzheimer's disease, early Alzheimer's disease, mild cognitive impairment, cerebral amyloid angiopathy, frontotemporal dementia, dementia with Lewy bodies, Lewy body dementia, Parkinson's disease, vascular dementia, limbic-predominant age-related TDP-43 encephalopathy, frontotemporal lobar degeneration, corticobasal degeneration, Pick's disease, multiple system atrophy, and progressive supranuclear palsy. In some embodiments, the subject in need thereof has demonstrated evidence of at least one disease chosen from Alzheimer's disease, pre-Alzheimer's disease, and early Alzheimer's disease. In some embodiments, the subject in need thereof has demonstrated evidence of mild cognitive impairment. In some embodiments, the subject in need thereof has demonstrated evidence of cerebral amyloid angiopathy, frontotemporal dementia, dementia with Lewy bodies, Lewy body dementia, vascular dementia, limbic-predominant age-related TDP-43 encephalopathy, frontotemporal lobar degeneration, corticobasal degeneration. In some embodiments, the subject in need thereof has demonstrated evidence of at least one disease chosen from Parkinson's disease, Pick's disease, multiple system atrophy, and progressive supranuclear palsy.

In some embodiments, the reduction of neurodegeneration is observed by maintenance or slowing of reduction of the thickness of the cortex relative to a subject to whom placebo was administered. In some embodiments, the reduction of neurodegeneration is observed by maintenance or slowing of reduction of the thickness of the cortex relative to a subject's baseline. In some embodiments, the reduction of neurodegeneration is observed by maintenance or slowing of reduction of the size of the hippocampus. In some embodiments, the reduction of neurodegeneration is observed by the preservation or reduction of loss of pyramidal neuronal cells relative to a subject's baseline or to a subject to whom placebo was administered. In some embodiments, the reduction of neurodegeneration is observed by the preservation or reduction of loss of granule neuronal cells relative to a subject's baseline or to a subject to whom placebo was administered.

Methods for measuring the thickness of portions of the brain such as the cortex or measuring the size of the hippocampus can be achieved using magnetic resonance imaging (MRI). High spatial resolution sMRI now allows for volumetry of hippocampal subfields. Early changes in CA1 have been observed in AD, with volumetric studies indicating that CA1 atrophy measures may improve diagnostic accuracy at the MCI stage. Novel MRI techniques, such as quantitative susceptibility mapping (QSM) or the T2* transverse relaxation time, have shown that iron levels and its rate of accumulation are heterogeneous in the human brain and correlates with cognitive impairment and slowing of motor performance. Neuronal dysfunction and altered connectivity of distinct brain networks are thought to occur early in the course of neurodegenerative diseases and can be measured indirectly with functional magnetic resonance imaging (fMRI). The whole brain or at least one area of the brain (for example, cortical gray matter (i.e., cortex), lateral ventricles, frontal lobe, parietal lobe, temporal lobe, occipital lobe, cingulate cortex, amygdala, piriform cortex, entorhinal cortex, hippocampus, hippocampal CA3 (pyramidal neurons), and/or hippocampal dentate gyrus (granule cell neurons) can be analyzed by MRI. In some embodiments, neurodegeneration is reduced within 48 hours of administration of the first dose of lemborexant to the subject relative to the subject's baseline. In some embodiments, the amount of p-tau is reduced within 24 hours of administration of the first dose of lemborexant to the subject relative to the subject's baseline. In some embodiments, neurodegeneration is reduced within 12 hours of administration of the first dose of lemborexant to the subject relative to the subject's baseline. In some embodiments, neurodegeneration is reduced within 6 hours of administration of the first dose of lemborexant to the subject relative to the subject's baseline.

In some embodiments, neurodegeneration is reduced or maintained for at least 30 days after administration of the first dose of lemborexant. In some embodiments, the amount of p-tau in a subject's CSF is reduced or maintained for at least 30 days after administration of the first dose of lemborexant.

In some embodiments, neurodegeneration is reduced within 1, 2, 3, 4, 5, 10, 15, 20, 25, 30, 35, 40, 45, 50, or 55 days of administration of the first dose of lemborexant. In some embodiments, the amount of p-tau in a subject's CSF is reduced for at least 1, 2, 3, 4, 5, 10, 15, 20, 25, 30, 35, 40, 45, 50, or 55 days after administration of the first dose of lemborexant.

In some embodiments, neurodegeneration is reduced or maintained within 30 days of administration of the first dose of lemborexant. In some embodiments, the amount of p-tau in a subject's CSF is reduced or maintained for at least 30 days after administration of the first dose of lemborexant. In some embodiments, neurodegeneration is reduced or maintained within 60 days of administration of the first dose of lemborexant.

In some embodiments, neurodegeneration is reduced or maintained within 45 days of administration of the first dose of lemborexant. In some embodiments, the amount of p-tau in a subject's CSF is reduced or maintained for at least 45 days after administration of the first dose of lemborexant.

In some embodiments, neurodegeneration is reduced or maintained within 60 days of administration of the first dose of lemborexant. In some embodiments, the amount of p-tau in a subject's CSF is reduced or maintained for at least 60 days after administration of the first dose of lemborexant.

In some embodiments, neurodegeneration is reduced or maintained for at least 120 days after administration of the first dose of lemborexant. In some embodiments, the amount of p-tau in a subject's CSF is reduced or maintained for at least 120 days after administration of the first dose of lemborexant.

In some embodiments, neurodegeneration is reduced or maintained for at least 180 days after administration of the first dose of lemborexant. In some embodiments, the amount of p-tau in a subject's CSF is reduced or maintained for at least 180 days after administration of the first dose of lemborexant.

In some embodiments, the effects of lemborexant on neurodegeneration or p-tau are apparent after a period of time, for example, after 3 months, after 6 months, or after 9 months of treatment. In some embodiments, neurodegeneration begins to reduce after at least 6 months of treatment. In some embodiments, neurodegeneration is reduced after at least 3 months of treatment, for example, after 6 months or after 9 months of treatment. In some embodiments, the amount of p-tau in a subject's CSF is reduced or maintained after a period of at least 3 months. In some embodiments, the amount of p-tau in a subject's CSF is reduced or maintained after a period of at least 6 months after treatment or a period of at least 9 months of treatment. In some embodiments, neurodegeneration is reduced or maintained for at least 6 months after administration of the first dose of lemborexant. In some embodiments, the amount of p-tau in a subject's CSF is reduced or maintained for at least 6 months after administration of the first dose of lemborexant.

In some embodiments, neurodegeneration is reduced for at least 1 year after administration of the first dose of lemborexant. In some embodiments, the amount of p-tau in a subject's CSF is reduced for at least 1 year after administration of the first dose of lemborexant. In some embodiments, the amount of t-tau in a subject's CSF is reduced for at least 1 year after administration of the first dose of lemborexant. In some embodiments, the amount of Aβ in a subject's CSF is reduced for at least 1 year after administration of the first dose of lemborexant. In some embodiments, the amount of fibrillar plaques is reduced for at least 1 year after administration of the first dose of lemborexant. In some embodiments, the amount of activated microglia cells is increased for at least 1 year after administration of the first dose of lemborexant.

### A. Altering Neurodegeneration

Accordingly, one aspect of the present disclosure relates to a method of altering neurodegeneration (e.g., reducing or delaying neurodegeneration, or slowing a rate of growth thereof), in a subject having AD or at risk of developing AD, comprising administering to the subject a therapeutically effective amount of lemborexant, a pharmaceutically acceptable salt thereof, or a solvate thereof, wherein the therapeutically effective amount is sufficient to alter neurodegeneration in the subject.

In some embodiments, altering neurodegeneration comprises slowing progression of, slowing a rate of progression of, delaying progression of, arresting development of, and reversing progression of neurodegeneration. In some embodiments, altering neurodegeneration comprises preventing the onset of or preventing development of pathology resulting from or coinciding with neurodegeneration). Altering neurodegeneration may reduce, delay, or slow a rate of onset and/or progression of symptoms of this pathology. In some embodiments, altering neurodegeneration comprises relieving or ameliorating one or more symptoms of neurodegeneration and/or improving one or more clinical metrics of pathology resulting from or coinciding with neurodegeneration (e.g., cognitive function, brain amyloid or tau levels, and/or expression of a biomarker). In some embodiments, altering neurodegeneration comprises preventing occurrence or reoccurrence of one or more symptoms of neurodegeneration.

In some embodiments, the subject is amyloid negative. The subject may have mild cognitive impairment or mild dementia. In some embodiments, the subject does not show signs of dementia and/or cognitive impairment.

In some embodiments, the neurodegeneration is altered relative to a reference. Accordingly, altering neurodegeneration may comprise reducing and/or delaying neurodegeneration, and/or slowing the rate thereof, as compared to a reference. In some embodiments, the reference is a baseline measurement from the subject prior to treatment. In some embodiments, the reference is a baseline measurement from a control subject. The reference may be a measurement obtained from more than one control subjects, which is used as a standard or threshold measurement. In some embodiments, the reference is a measurement from a control subject administered a placebo.

In some embodiments, neurodegeneration is characterized by at least one of a loss of cortical thickness or a reduction in hippocampal volume. In some embodiments, altering neurodegeneration comprises maintaining or slowing a reduction of cortical thickness in the subject. In some embodiments, neurodegeneration is characterized by at least one of loss of a pyramidal neurons in the cortex or a loss of pyramidal or granule neurons in the hippocampus. In some embodiments, altering neurodegeneration comprises maintaining or slowing a reduction of hippocampal volume in the subject. In some embodiments, altering neurodegeneration comprises maintaining or reducing loss of pyramidal neurons or granule neurons. In some embodiments, altering neurodegeneration comprises reducing a rate of neurodegeneration. In some embodiments, altering neurodegeneration comprises altering a neurofilament light chain (NfL) level. NfL levels may be altered in the blood and/or CSF of the subject.

### VIII. Selecting a Subject for Treatment

A further aspect of the present disclosure relates to a method of selecting a subject having Alzheimer's disease (AD) or at risk of developing AD for treatment with lemborexant, a pharmaceutically acceptable salt thereof, or a solvate thereof, comprising: (a) obtaining from the subject a measurement of at least one of tau phosphorylation, tau aggregation, neurodegeneration, Aβ plaque burden, microglial response, and biomarker expression; (b) comparing the measurement from the subject to a measurement from a reference; and (c) selecting the subject for treatment with lemborexant if the measurement from the subject differs from the measurement from the reference.

In some embodiments, the subject has mild cognitive impairment or mild dementia. In some embodiments, the subject does not show signs of dementia and/or cognitive impairment.

In some embodiments, the subject is at risk for further Aβ accumulation. The subject may be at risk for spread of tau pathology. The subject may be at risk for cognitive decline. In some embodiments, the subject may have intermediate levels of amyloid PET (e.g., approximately 20-40 centiloids). In some embodiments, the subject may have elevated levels of amyloid PET (e.g., >40 centiloids). In some embodiments, the subject may be an ApoE4 carrier. In some embodiments, the subject may have one or more risk factors for developing AD, such as having a family history with a first degree relative having AD or dementia, having an age of 65 years or older, being female, having or recovering from traumatic brain injury, and suffering from other conditions such as obesity, diabetes, heart disease, and/or blood vessel disease.

In some embodiments, the subject has early-stage AD. In some embodiments, the subject has pre-AD. In some embodiments, the subject has been diagnosed with AD, based on brain imaging, cognitive function, and/or biomarker criteria.

In some embodiments, obtaining at least one measurement comprises obtaining data from a brain scan of the subject and/or obtaining data from a biological sample from the subject. In some embodiments, the data from the brain scan may indicate a level of tau phosphorylation, tau aggregation, Aβ plaque burden, and/or microglial response.

In some embodiments, the biological sample is a body fluid. In some embodiments, the body fluid is cerebrospinal fluid (CSF), blood, or saliva.

In some embodiments, the reference is a control. In some embodiments, the reference is a baseline measurement from a control subject. The reference may be a measurement obtained from more than one control subjects, which is used as a standard or threshold measurement. In some embodiments, the reference is a measurement from a control subject administered a placebo.

In some embodiments, the control does not have AD. The measurement from the subject may be higher than the measurement from the control who does not have AD. The measurement from the subject may be lower than the measurement from the control who does not have AD.

In some embodiments, the control has AD. The measurement from the subject is comparable to or higher than the measurement from the control who has AD. The measurement from the subject may be comparable to or lower than the measurement from the control who has AD.

In some embodiments, the measurement of tau phosphorylation comprises a measurement of phosphorylation on one more of T181, T217, S202, or S205.

In some embodiments, the measurement of tau aggregation comprises a measurement of insoluble tau aggregates (e.g., neurofibrillary tangles (NFTs)).

In some embodiments, the measurement of neurodegeneration comprises a measurement of cortical thickness and/or hippocampal volume or a measurement of loss of pyramidal neurons or granule neurons.

In some embodiments, wherein the measurement of Aβ plaque burden comprises a measurement of Aβ plaque volume and/or growth of Aβ plaque volume.

In some embodiments, the measurement of Aβ plaque burden comprises a measurement of amyloid PET signal in a brain region of the subject or a measurement of Aβ in the CSF of the subject.

In some embodiments, the measurement of microglial response is a change in the expression of at least one microglial marker. The microglial marker may be Iba1, Clec7a, CD68, TMEM119, or P2RY12. In some embodiments, the measurement of microglial response is a measurement of phagocytosis by microglia.

### IX. Treatment Efficacy

Disclosed herein are methods of treating a subject with lemborexant, a pharmaceutically acceptable salt thereof, or a solvate thereof, and methods for monitoring treatment efficacy of the subject.

One aspect of the present disclosure relates to a method of monitoring treatment efficacy in a subject having Alzheimer's disease (AD) or at risk for developing AD, comprising: (a) obtaining from the subject a first measurement of at least one of tau phosphorylation, tau aggregation, neurodegeneration, Aβ plaque burden, microglia function, and biomarker expression; (b) administering to the subject a dose of lemborexant, a pharmaceutically acceptable salt thereof, or a solvate thereof; (c) obtaining from the subject a second measurement of at least one of tau phosphorylation, tau aggregation, neurodegeneration, Aβ plaque burden, microglia function, and biomarker expression; and (d) comparing the second measurement from the subject to the first measurement from the subject, wherein a difference between the first measurement and the second measurement indicates effective treatment with lemborexant.

Another aspect of the present disclosure relates to a method of treating a subject having Alzheimer's disease (AD) or at risk for developing AD, comprising: (a) obtaining from the subject a first measurement of at least one of tau phosphorylation, tau aggregation, neurodegeneration, Aβ plaque burden, microglial response, and biomarker expression; (b) administering to the subject a first dose of lemborexant, a pharmaceutically acceptable salt thereof, or a solvate thereof; (c) obtaining from the subject a second measurement of at least one of tau phosphorylation, tau aggregation, neurodegeneration, Aβ plaque burden, microglia function, and biomarker expression; (d) comparing the second measurement from the subject to the first measurement, and (d) administering a second dose of lemborexant if the first measurement differs from the second measurement in the comparison measurement. The first and second measurements may differ in that the second measurement is higher than the first. Alternatively, the second measurement may be lower than the first. For example, if the measurement is microglial response, in some embodiments, the first measurement of microglial response may be higher than the second measurement of microglial response.

Still a further aspect of the present disclosure relates to a method of monitoring treatment efficacy in a subject having Alzheimer's disease (AD) or at risk for developing AD, comprising: (a) obtaining from the subject a first measurement of at least one of tau phosphorylation, tau aggregation, neurodegeneration, Aβ plaque burden, microglia function, and biomarker expression; (b) administering to the subject a dose of lemborexant, a pharmaceutically acceptable salt thereof, or a solvate thereof; (c) obtaining from the subject a second measurement of at least one of tau phosphorylation, tau aggregation, neurodegeneration, Aβ plaque burden, microglia function, and biomarker expression; and (d) comparing the second measurement from the subject to the first measurement from the subject to obtain a comparison measurement, wherein a difference between the first measurement and the second measurement in the comparison measurement or a difference between the comparison measurement and a reference measurement indicates effective treatment with lemborexant.

Another aspect of the present disclosure relates to a method of treating a subject having Alzheimer's disease (AD) or at risk for developing AD, comprising: (a) obtaining from the subject a first measurement of at least one of tau phosphorylation, tau aggregation, neurodegeneration, Aβ plaque burden, microglial response, and biomarker expression; (b) administering to the subject a first dose of lemborexant, a pharmaceutically acceptable salt thereof, or a solvate thereof; (c) obtaining from the subject a second measurement of at least one of tau phosphorylation, tau aggregation, neurodegeneration, Aβ plaque burden, microglia function, and biomarker expression; (d) comparing the second measurement from the subject to the first measurement from the subject to obtain a comparison measurement, and (e) administering a second dose of lemborexant if the first measurement differs from the second measurement in the comparison measurement, or if the comparison measurement differs from a reference measurement.

In some embodiments, obtaining at least one measurement comprises obtaining data from a brain scan of the subject and/or obtaining data from a biological sample from the subject. In some embodiments, the data from the brain scan indicates a level of tau phosphorylation, tau aggregation, Aβ plaque burden, and/or microglial response. In some embodiments, the biological sample is a body fluid. In some embodiments, the body fluid is cerebrospinal fluid (CSF), blood, or saliva.

In some embodiments, the first measurement from the subject is higher than the second measurement from the subject. In some embodiments, the first measurement from the subject is lower than the second measurement from the subject.

In some embodiments, for example, in those methods comprising a comparison measurement and a reference measurement, the reference measurement is obtained from at least one control. In some embodiments, the reference measurement is a comparison of a first measurement from a control and a second measurement from a control. In some embodiments, the comparison measurement is higher than the reference measurement. In some embodiments, the comparison measurement is lower than the reference measurement. For example, the comparison measurement, which compares a first measurement from the subject and a second measurement from the subject, may indicate that no change has occurred. The reference measurement, meanwhile, may indicate that a change occurred in a control. Accordingly, the difference between the comparison measurement and the reference measurement may indicate whether a treatment has been efficacious and/or whether a second dose of lemborexant should be administered.

In some embodiments, the reference measurement is a measurement from a control. The reference measurement may be obtained from more than one control subjects, which is used as a standard or threshold measurement. In some embodiments, the reference measurement is a measurement from a control subject administered a placebo.

In some embodiments, the control does not have AD. In some embodiments, the comparison measurement is higher than the reference measurement. In some embodiments, the comparison measurement is lower than the reference measurement.

In some embodiments, the control has AD, for example, untreated AD. In some embodiments, the comparison measurement is higher than the reference measurement. In some embodiments, the comparison measurement is lower than the reference measurement.

In some embodiments, the measurement of tau phosphorylation comprises a measurement of phosphorylation of one or more of T181, T217, S202, S205, or T231.

In some embodiments, the measurement of tau aggregation comprises a measurement of insoluble tau aggregates (e.g., neurofibrillary tangles (NFTs)).

In some embodiments, the measurement of neurodegeneration comprises a measurement of cortical thickness and/or hippocampal volume or a measurement of loss of pyramidal neurons or granule neurons.

In some embodiments, the measurement of Aβ plaque burden comprises a measurement of Aβ plaque volume and/or growth of Aβ plaque volume.

In some embodiments, the measurement of Aβ plaque burden comprises a measurement of amyloid PET signal in a brain region of the subject or a measurement of Aβ in the CSF of the subject.

In some embodiments, the measurement of microglial response is a measure of expression of at least one microglial marker. The microglial marker may be Iba1, Clec71, P2RY12 or TMEM 119. In some embodiments, the measurement of microglial response is a measurement of phagocytosis by microglia.

In some embodiments, the measurement of a biomarker expression is a measurement of Ifnb1, MMP2, and/or Bace1 expression.

In some embodiments, the subject is amyloid-negative.

In some embodiments, the subject has Aβ plaques.

In some embodiments, the subject has mild cognitive impairment or mild dementia. In some embodiments, the subject does not show signs of dementia and/or cognitive impairment.

In some embodiments, the subject is at risk for further Aβ accumulation. The subject may be at risk for spread of tau pathology. The subject may be at risk for cognitive decline. In some embodiments, the subject may have intermediate levels of amyloid PET (e.g., approximately 20-40 centiloids). In some embodiments, the subject may have elevated levels of amyloid PET (e.g., >40 centiloids). In some embodiments, the subject may be an ApoE4 carrier. In some embodiments, the subject may have one or more risk factors for developing AD, such as having a family history with a first degree relative having AD or dementia, having an age of 65 years or older, being female, having or recovering from traumatic brain injury, and suffering from other conditions such as obesity, diabetes, heart disease, and/or blood vessel disease.

In some embodiments, wherein the subject has early-stage AD. In some embodiments, the subject has pre-AD. In some embodiments, the subject has been diagnosed with AD, based on brain imaging, cognitive function, and/or biomarker criteria.

### X. Dosages

A disclosed herein, a dose of lemborexant may refer to a therapeutically effective amount of lemborexant, a pharmaceutically acceptable salt thereof, or a solvate thereof. In some embodiments, the methods disclosed herein comprise administering orally 5 mg to 20 mg of lemborexant to the subject once per day. In some embodiments, one dose of 20 mg of lemborexant is administered to the subject once per day. In some embodiments, one dose of 25 mg of lemborexant is administered to the subject once per day. In some embodiments, one dose of 25 mg of lemborexant is administered to the subject once per day for at least 2 days. In some embodiments, one dose of 25 mg of lemborexant is administered to the subject once per day. In some embodiments, one dose of 25 mg of lemborexant is administered to the subject once per day for at least 5 days. In some embodiments, one dose of 25 mg of lemborexant is administered to the subject once per day. In some embodiments, one dose of 25 mg of lemborexant is administered to the subject once per day for at least one week. In some embodiments, one dose of 25 mg of lemborexant is administered to the subject once per day. In some embodiments, one dose of 25 mg of lemborexant is administered to the subject once per day for at least one month.

In some embodiments, the methods disclosed herein comprise administering orally a dosage form comprising lemborexant to the subject. In some embodiments, the methods disclosed herein comprise administering orally a dosage form comprising 10 mg of lemborexant to the subject. In some embodiments, the methods disclosed herein comprise administering orally a dosage form comprising 15 mg of lemborexant to the subject. In some embodiments, the methods disclosed herein comprise administering orally a dosage form comprising 20 mg of lemborexant to the subject. In some embodiments, the methods disclosed herein comprise administering orally a dosage form comprising 25 mg of lemborexant to the subject. In some embodiments, the methods disclosed herein comprise administering orally a dosage form comprising 30 mg of lemborexant to the subject. In some embodiments, the methods disclosed herein comprise administering orally a dosage form comprising 35 mg of lemborexant to the subject. In some embodiments, the methods disclosed herein comprise administering orally a dosage form comprising 40 mg of lemborexant to the subject. In some embodiments, the methods disclosed herein comprise administering orally a dosage form comprising 45 mg of lemborexant to the subject. In some embodiments, the methods disclosed herein comprise administering orally a dosage form comprising 50 mg of lemborexant to the subject.

Dosage forms of the present disclosure comprise lemborexant in a therapeutically effective amount for treatment of when administered in accordance with the teachings of the present disclosure. The unit dose of the effective amount in a dosage form is from 0.5 mg to 100 mg, from 2 mg to 75 mg, from 2 mg to 70 mg, from 2 mg to 65 mg, from 2 mg to 60 mg, from 2 mg to 55 mg, from 2 mg to 50 mg, from 2 mg to 45 mg, from 2 mg to 40 mg, from 2 mg to 35 mg, from 2 mg to 30 mg, from 2 mg to 25 mg, from 2 mg to 20 mg, from 2 mg to 15 mg, from 2 mg to 15 mg, 2 mg, 2.5 mg, 4 mg, 5 mg, 8 mg, 10 mg, 15 mg, 20 mg, 25 mg, 30 mg, 35 mg, 40 mg, 45 mg, or 50 mg. The unit dose is not limited by the type of the dosage form or the number of dosage forms for single dose. In some embodiments, the unit dose may be 2.5 mg. In some embodiments, the unit dose may be 5 mg. In some embodiments, the unit dose may be 10 mg. In some embodiments, the unit dose may be 7.5 mg. In some embodiments, the unit dose may be 12.5 mg. In some embodiments, the unit dose may be 15 mg. In some embodiments, the unit dose may be 18 mg. In some embodiments, the unit dose may be 20 mg. In some embodiments, the unit dose may be 22 mg. In some embodiments, the unit dose may be 25 mg. In some embodiments, the unit dose may be 30 mg. In some embodiments, the unit dose may be 32 mg. In some embodiments, the unit dose may be 35 mg. In some embodiments, the unit dose may be 40 mg. In some embodiments, the unit dose may be 50 mg.

Accordingly, a therapeutically effective amount of lemborexant, a pharmaceutically acceptable salt thereof, or a solvate thereof, as administered herein, may comprise a dosage falling within a range, for example, a range of 5 mg to 50 mg per day.

In some embodiments, the therapeutically effective amount of lemborexant administered to the subject is in a range of 5 mg to 50 mg per day. For example, the therapeutically effective amount of lemborexant administered to the subject may be in a range of 10 mg to 30 mg per day. In some embodiments, the therapeutically effective amount of lemborexant administered to the subject is selected from 5 mg, 7.5 mg, 10 mg, 12.5 mg, 15 mg, 17.5 mg, 20 mg, 22.5 mg, 25 mg, 27.5 mg and 30 mg per day.

In some embodiments, the therapeutically effective amount of lemborexant administered to the subject is 20-25 mg per day.

In some embodiments, one dose of 20 mg of lemborexant is administered to the subject once per day.

In some embodiments, lemborexant is administered at a first dose for a first period, a second dose for a second period, and optionally, at a third dose for a third period. Each of the first period, the second period, and the third period may be 1 week.

In some embodiments, the first dose is lower than the second dose, and optionally, the second dose is lower than the third dose. For example, in some embodiments, the first dose is 5 mg of lemborexant once per day, the second dose is 10 mg of lemborexant once per day, and, optionally, the third dose is 20-25 mg of lemborexant once per day. In some embodiments, the first dose is 5 mg or 7.5 mg lemborexant once per day, the second dose is 10 mg, 12.5 mg, 15 mg, or 17.5 mg of lemborexant once per day, and the third dose is 20 mg, 22.5 mg, 25 mg, 27.5 mg or 30 mg of lemborexant once per day.

In some embodiments, the first dose is higher than the second dose, and optionally, the second dose is higher than the third dose. For example, in some embodiments, the first dose is 20-25 mg of lemborexant once per day, the second dose is 10 mg of lemborexant once per day, and, optionally, the third dose is 5 mg of lemborexant once per day. In some embodiments, the first dose is 20 mg, 22.5 mg, 25 mg, 27.5 mg or 30 mg of lemborexant once per day, the second dose is 10 mg, 12.5 mg, 15 mg, or 17.5 mg of lemborexant once per day, and, optionally, the third dose is 5 mg or 7.5 mg of lemborexant once per day.

In some embodiments, lemborexant may be administered to the subject over a period of time. In some embodiments, the methods described herein comprise administering lemborexant to the subject for at least 6 months. In some embodiments, the methods described herein comprise administering lemborexant to the subject for at least 9 months, at least 12 months, or at least 15 months. In some embodiments, the methods described herein comprise administering lemborexant to the subject for at least 18 months. In some embodiments, the methods described herein comprise administering lemborexant to the subject for at least 24 months, 30 months, or 36 months. In some embodiments, lemborexant may be administered for the remainder of the subject's life.

### XI. Pharmaceutical Compositions

In some embodiments, a dosage form of the present disclosure may constitute one or more pharmaceutical compositions comprising lemborexant together with pharmaceutically acceptable excipients.

As used herein, the term “composition” used herein includes a product comprising a particular ingredient in a particular amount and any product directly or indirectly brought about by the combination of particular ingredients in particular amounts. Such a term related to the pharmaceutical composition is intended to include a product comprising an active ingredient and an inert ingredient constituting a carrier and include every product directly or indirectly brought about by the combination, complexation or aggregation of any two or more ingredients or the dissociation, other kinds of reactions or interaction of one or more ingredients. Thus, the pharmaceutical composition of the present disclosure includes every composition prepared by mixing the compound of the present disclosure with a pharmaceutically acceptable carrier.

As used herein, the term “pharmaceutically acceptable” means that a carrier, diluent, excipient, or vehicle is compatible with other components of a formulation and is nontoxic to a subject.

Solid dosage forms of the present disclosure include capsules, granules, lozenges, pellets, pills, powders, suspensions, and tablets.

The pharmaceutical compositions of the present disclosure may be prepared using standard techniques and manufacturing processes generally known in the art. See, e.g., the monograph of Japanese Pharmacopoeia, 16th Edition; and Pharmaceutical Dosage Forms of U.S. Pharmacopoeia-NF, Chapter 1151.

In some embodiments, pharmaceutical compositions comprise lemborexant. In some embodiments, pharmaceutical compositions further comprise at least one additional component chosen from pharmaceutically acceptable carriers, pharmaceutically acceptable vehicles, and pharmaceutically acceptable excipients.

In some embodiments, the at least one additional component in the pharmaceutical compositions is chosen depending upon the route of administration for which the pharmaceutical composition is intended. Non-limiting examples of suitable routes of administration for which the pharmaceutical composition may be used include parenteral, oral, inhalation spray, topical, rectal, nasal, buccal, vaginal and implanted reservoir administration. The term “parenteral” as used herein includes subcutaneous, intravenous, intramuscular, intra-articular, intra-synovial, intrasternal, intracisternal, intrathecal, intrahepatic, intralesional and intracranial injection or infusion techniques. In some embodiments, the mode of administration is chosen from intravenous, oral, subcutaneous, and intramuscular administration. Sterile injectable forms of the compositions of this disclosure may be, for example, aqueous or oleaginous suspension. These suspensions may be formulated according to techniques known in the art using suitable dispersing or wetting agents and suspending agents known in the art. The sterile injectable preparation may also be a sterile injectable solution or suspension in a nontoxic parenterally acceptable diluent or solvent, for example, as a solution in 1,3-butanediol. Non-limiting examples of vehicles and solvents that may be employed include water, Ringer's solution, and isotonic sodium chloride solution. In addition, sterile, fixed oils may be employed as a solvent and/or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, and/or other dosage forms, may also be used for the purposes of formulation.

For oral administration, lemborexant, may be provided in an acceptable oral dosage form, including, but not limited to, suspensions, capsules, tablets, oral disintegrating tablets, sprinkles, and other oral formulations that would be easy to swallow. In some embodiments, lemborexant is provided in the form of tablet or capsules. In some embodiments, lemborexant is provided in the form of crushable tablets. In the case of tablets for oral use, carriers commonly used include lactose and cornstarch. Lubricating agents, such as magnesium stearate, may also be added. For oral administration in a capsule form, useful diluents include lactose and dried cornstarch. When aqueous suspensions are required for oral use, the active ingredient is combined with an emulsifying and/or suspending agent. If desired, certain sweetening, flavoring or coloring agents may also be added.

In order that the disclosure described herein may be more fully understood, the following examples are set forth. It should be understood that these examples are for illustrative purposes only and are not to be construed as limiting this disclosure in any manner.

### NON-LIMITING EMBODIMENTS OF THE DISCLOSURE

1. A method of reducing or maintaining the amount of p-tau or t-tau in a subject, the method comprising administering to a subject in need thereof a therapeutically effective amount of lemborexant.
2. The method of embodiment 1, wherein the amount of p-tau or t-tau in a subject is reduced or maintained relative to the subject's baseline.
3. The method of embodiment 1 or 2, wherein the amount of p-tau or t-tau in a subject is reduced or maintained relative to placebo.
4. The method of any one of embodiments 1-3, wherein the ratio of p-tau to tau in the subject is lower after the administration of a therapeutically effective amount of lemborexant relative to the subject's baseline or relative to placebo.
5. The method of any one of embodiments 1-4, wherein the rate of dephosphorylation of tau is higher in the subject after the administration of a therapeutically effective amount of lemborexant relative to the subject's baseline or relative to placebo.
6. The method of any one of embodiments 1-5, wherein the rate of tau phosphorylation in the subject is lower after the administration of a therapeutically effective amount of lemborexant relative to the subject's baseline or relative to placebo.
7. The method of any one of embodiments 1-6, wherein the ratio of p-tau/t-tau in the subject's CSF is reduced compared to the ratio of CSF p-tau/t-tau of the subject prior to administration of lemborexant.
8. The method of embodiment 7, wherein the ratio of p-tau/t-tau in the subject's CSF is maintained within 10% of the ratio of p-tau/t-tau of the subject prior to administration of lemborexant.
9. The method of any one of embodiments 1-8, wherein concentration of Aβ in the subject's CSF is lower than or the same as the concentration of Aβ in the subject's CSF prior to administration of lemborexant.
10. The method of embodiment 9, wherein the amyloid PET signal in the brain of the subject to whom a therapeutically effective amount of lemborexant is administered is lower than or the same as the amyloid PET signal in the brain of the subject prior to administration of lemborexant.
11. The method of any one of embodiments 1-10, wherein the tau PET signal in the subject's brain is decreased relative to baseline.
12. The method of any one of embodiments 1-11, wherein the tau is p-tau, t-tau, or aggregated tau.
13. The method of any one of embodiments 1-12, wherein p-tau and t-tau are reduced.
14. The method of any one of embodiments 1-13, wherein the reduction of tau phosphorylation occurs in the hippocampus, entorhinal cortex, and/or piriform cortex.
15. The method of any one of embodiments 1-14, wherein the Aβ in the subject's CSF is Aβ38, Aβ40, and/or Aβ42.
16. The method of any one of embodiments 1-15, wherein the amount of p-tau is reduced within 48 hours of administration of the first dose of lemborexant to the subject relative to the subject's baseline.
17. A method of reducing neurodegeneration in a subject, the method comprising administering to a subject in need thereof a therapeutically effective amount of lemborexant.
18. The method of embodiment 17, wherein the reduction of neurodegeneration is observed by maintenance or slowing of reduction of the thickness of the cortex relative to a subject's baseline or to a subject to whom placebo was administered.
19. The method of embodiments 17 or 18, wherein the reduction of neurodegeneration is observed by maintenance or slowing of reduction of the size of the hippocampus relative to a subject's baseline or to a subject to whom placebo was administered.
20. The method of any one of embodiments 17-19, wherein the reduction of neurodegeneration is observed by preservation or reduction of loss of pyramidal neuronal cells relative to a subject's baseline or to a subject to whom placebo was administered.
21. The method of any one of embodiments 17-20, wherein the reduction of neurodegeneration is observed by preservation or reduction of loss of granule neuronal cells relative to a subject's baseline or to a subject to whom placebo was administered.
22. The method of any one of embodiments 1-21, wherein the therapeutically effective amount of lemborexant administered to the subject is in a range of 10 mg to 50 mg per day.
23. The method of embodiment 22, wherein the therapeutically effective amount of lemborexant administered to the subject is in a range of 15 mg to 30 mg per day.
24. The method of embodiment 22, wherein the therapeutically effective amount of lemborexant administered to the subject is 25 mg per day.
25. The method of any one of embodiments 1-24, wherein one dose of 25 mg of lemborexant is administered to the subject once per day.
26. The method of embodiment 25, wherein one dose of 25 mg of lemborexant is administered to the subject once per day for at least two days.
27. The method of any one of embodiments 1-26, wherein one dose of 2.5 mg, 5 mg, 10 mg, 15 mg, or 20 mg of lemborexant is administered to the subject once per day after at least two days of administration of one dose of 25 mg of lemborexant once per day.
28. A method of reducing the concentration of Aβ in a subject comprising administering to a subject in need thereof a therapeutically effective amount of lemborexant.
29. The method of any one of embodiments 1-28, wherein the concentration of Aβ in the subject's CSF is lower than the concentration of Aβ in the subject's CSF prior to the administration of lemborexant.
30. The method of any one of embodiments 1-29, wherein the amyloid PET signal in the subject's brain of the subject to whom lemborexant is administered is lower than the amyloid PET signal in the brain of a subject prior to administration of lemborexant.
31. The method of embodiment 30, wherein the Aβ in the subject's CSF is Aβ38, Aβ40, and/or Aβ42.
32. The method of any one of embodiments 1-29, wherein the concentration of Aβ is reduced within 48 hours of the administering of lemborexant.
33. A method of increasing the number of activated microglial cells in a subject, the method comprising administering to a subject in need thereof a therapeutically effective amount of lemborexant.
34. The method of embodiment 33, wherein the number of activated microglial cells is increased relative to baseline.
35. The method of any one of embodiments 33 or 34, wherein the activated microglial cells are phagocytic microglial cells.
36. The method of any one of embodiments 33-35, wherein the number of activated microglial cells is measured by PET or CSF biomarkers of microglia activation.
37. The method of any one of embodiments 33-36, wherein the whole brain or at least one area of the brain is analyzed by PET.
38. The method of embodiment 37, wherein the at least one area of the brain analyzed by PET is chosen from cortical gray matter, lateral ventricles, frontal lobe, parietal lobe, temporal lobe, occipital lobe, cingulate cortex, amygdala, piriform cortex, entorhinal cortex, hippocampus, hippocampal CA3 (pyramidal neurons), and hippocampal dentate gyrus (granule cell neurons).

### EXAMPLES

### Example 1 Clinical Study Protocols

a. Trial 1: Acute Effects of Lemborexant on CSF Amyloid-Beta and Tau

The acute effects of lemborexant will be investigated in cognitively normal, amyloid-positive participants.

Inclusion criteria will be:
Age 60-80 years
Any sex
Any race/ethnicity
Mini-Mental Status Examination score (MMSE) 27
Positive plasma Aβ test (i.e., amyloid-positive)
Pittsburgh Sleep Quality Index >5

Exclusion Criteria will be:
Cognitive impairment as determined by history of MMSE <27
Inability to speak or understand English
Any sleep disorders other than insomnia
   No history of moderate-to-severe sleep-disordered breathing and STOP-Bang score >5
   History or reported symptoms suggestive of restless legs syndrome, narcolepsy or other sleep disorders
   No more than mild sleep apnea (AHI <16) on PSG
Sleep schedule outside the range of bedtime 22:00-midnight
Contraindication to lumbar catheter (anticoagulants; bleeding disorder; allergy to lidocaine or disinfectant; prior central nervous system or lower back surgery)
Cardiovascular disease requiring medication except for controlled hypertension (PI discretion)
Stroke
Hepatic or renal impairment
Pulmonary disease (PI discretion)
Type 1 diabetes
HIV or AIDS
Neurologic or psychiatric disorder requiring medication (PI discretion)
Suicidal ideations
Alcohol or tobacco use (PI discretion)
Use of sedating medications (PI discretion)
Inability to get out of bed independently
In the opinion of the investigator, the participant should be excluded due to an abnormal physical examination.
Current pregnancy
Body Mass Index >35
c History of migraines (PI discretion)
History of drug abuse in the last 6 months
History or presence of any clinically significant medical condition, behavioral or psychiatric disorder (including suicidal ideation), or surgical history based on medical record or patient report that could affect the safety of the subject or interfere with study assessments or in the judgment of the PI participant is not a good candidate.
Urinary or fecal incontinence
Concurrently enrolled in another trial of an investigational drug or device

Twelve (or more) participants will be randomized to receive placebo (N=4 or more) or lemborexant 25 mg (N=8 or more).

Procedure: Randomized participants will be admitted in the early afternoon (Night 1). All participants will have their sleep monitored with unattended full-montage PSG (Tracklt™; Lifelines, Troy, IL) that will allow for sleep staging according to the gold standard American Academy of Sleep Medicine criteria and has already been used in similar studies to monitor sleep for 36-48 hours.

At approximately 20:00, a lumbar catheter and two IVs will be placed in each participant for collecting 6 ml of CSF every 2 hours for 48 hours. The lumbar catheter ports will be placed on the outside of the gown sleeve for easy access to minimize disturbance during fluid collection. Sampling start time will begin ~1 hour prior to the typical bedtime defined by sleep logs for each participant in order to allow for ¹³C₆-leucine infusion and frequent sampling of blood prior to bedtime. Six milliliters of blood will be collected at the following time points: 0 (baseline), 5 minutes, 10 minutes, 15 minutes, 30 minutes, 1 hour, 1.5 hours, 2 hours, 2.5 hours, 3 hours, 3.5 hours, 4 hours, 6 hours, 8 hours, 10 hours, 12 hours, 14 hours, 16 hours, 18 hours, 20 hours, 22 hours, 24 hours, 26 hours, 28 hours, 30 hours, 32 hours, 34 hours, 36 hours, 38 hours, 40 hours, 42 hours, 44 hours, 46 hours, and 48 hours (Table 1).

TABLE 1
Timeline of Catheter Placement and
Removal, Bedtime, and Sampling

| Hour | 0 | 1 | 2 | 4 | 6 | 8 | 10 | 12 | 14 | 16 | 18 | 20 | 22 | 24 |
|---|---|---|---|---|---|---|---|---|---|---|---|---|---|---|
| Event | C, | B | X | X | X | X | X | X | X | X | X | X | X | X |
| | X | | | | | | | | | | | | | |
| Hour | 25 | 26 | 28 | 30 | 32 | 34 | 36 | 38 | 40 | 42 | 44 | 46 | 48 | 49 |
| Event | B | X | X | X | X | X | X | X | X | X | X | X | X, | B |
| | | | | | | | | | | | | | R | |

C: catheter placement; B: bedtime; X: CSF/blood sampling; R: catheter removal

Approximately 1 hour before the habitual bedtime on Day 1 (t=0), all participants will start an infusion of 800 mg labeled ¹³C₆-leucine to label proteins in vivo during intracellular translation in order to monitor Aβ kinetics.

Participants will be allowed to sleep as they are able immediately with the lights turned off at their habitual bedtime (bedtime is at hour 1, approximately 22:00-00:00). Dim red lights (safelights) will be used when collecting CSF and blood in the dark. Participants will sleep until final awakening in the morning.

During Day 2, all participants will be in well-lit rooms with regular monitoring for staying awake and naps will not be permitted. At ~22:00 on Day 2 (hour 25), all participants will follow the same sleep routine as followed the previous night except that there will be no infusion of labeled ¹³C-leucine and the time of “Hour 26” will depend on the participant's regular bedtime. Participants will receive the same placebo or lemborexant dose as the previous night. The study will end at ~20:00 on Day 3 (hour 48) at which time the IVs and lumbar catheters will be removed. All participants will then sleep overnight and will be monitored for at least 8 hours after catheter removal and then discharged.

CSF tau, phosphorylated tau, and Aβ kinetics will be quantified by mass spectrometry. For the determination of baseline for each participant, AD biomarkers (Aβ38, Aβ40, Aβ42, T181, S202, T217, pT181, pS202, pT217) will be normalized to the average of the first 6 hours (t=0-6, 20:00-02:00) before changes from the intervention will be seen in CSF. The hour 0 concentration will also be normalized. Phosphorylated tau ratios (pT181/T181, pS202/S202, pT217/T217, p-tau/t-tau) will be normalized to the first time point (hour 0). The trajectory of changes from these baselines over time will then be plotted by treatment arms to examine whether the change in concentrations or ratios is linear.

For the statistical analysis, if the linearity assumption is valid, (linear mixed effects) LME models with random intercept and slope will be used for analysis, otherwise mixed model for repeated measure (MMRM) will be used. Fixed effect in the model will include treatment group, time and their interaction. Baseline will be included as a covariate. The normality assumption will be examined using residual plot and appropriate transformation (e.g., log) will be considered. Unstructured covariance matrix will be used and if there is convergence issue, various other covariance matrix structure (e.g., compound symmetry, First-order Autoregressive) will be compared and the best fit structure will be selected for final analysis based on Akaike information criterion (AIC).

b. Trial 2: Treatment of Subjects Having Preclinical or Early Alzheimer's Disease

This trial will evaluate the efficacy of lemborexant in the prevention or delay of Aβ accumulation, spread of downstream tau pathology, and cognitive decline across the continuum of preclinical and early AD.

Amyloid-ß (Aß) accumulation often begins more than a decade prior to the clinical stages of Alzheimer's disease (AD) and is thought to play a critical role in accelerating the spread of tauopathy and neurodegeneration during the preclinical stages of the disease. Multiple neuroimaging and biomarker observational studies demonstrate that Aβ accumulation is associated with increased risk of cognitive decline among clinically normal older individuals.

The trial will utilize NAV4694 (flutafuranol) amyloid PET imaging to assess fibrillar amyloid pathology for eligibility and longitudinal outcomes, and the MK6240 tau PET tracer to assess spread of neurofibrillary tangle and tau neurite pathology longitudinally. Clinical outcomes include the Preclinical Alzheimer Cognitive Composite-5 (PACC-5) composed of the Free and Cued Selective Reminding Test, Paragraph Recall Ila, Digit-Symbol, MMSE, and Semantic Category Fluency, as well as the Cognitive Function Index (CFI), a participant- and study-partner report of cognitive function.

In addition, scans of the patient's cortex and hippocampus will be performed to assess extent of neurodegeneration. CSF and blood will be collected to measure AD biomarkers such as Aβ, tau, p-tau, and NfL.

i. Group 1: Patients with Preclinical Alzheimer's Disease—Intermediate Levels of Amyloid

The goal of this trial is to determine if lemborexant treatment leads to primary prevention or delay of AD, through preventing or slowing early Aβ build-up in the brain. The trial will enroll cognitively-normal individuals with intermediate levels of amyloid on screening PET imaging (approximately 20-40 centiloids), thought to be in the earliest preclinical stages of AD who are at risk for further Aβ accumulation and early spread of tau pathology over four years.

Patient Inclusion Criteria. Patients will be selected for intact cognition, determined by a Mini-Mental State Examination (MMSE) score of 27 after education adjustment, with a global clinical dementia rating (CDR) of zero. Patients will have intermediate amyloid levels on PET imaging (20-40 centiloids).

Lemborexant administration. Lemborexant or matched placebo will be administered to participants every month during in-person study visits where treatment adherence and adverse events will be assessed. Lemborexant is approved by the FDA for the treatment of insomnia in doses from 5-10 mg. After an interim analysis for safety and efficacy, the lemborexant dose may be increased to 20 mg. At the start of the study, participants will take lemborexant 5 mg once per day for a 1-week period and then increased to lemborexant 10 mg once per day; there will be matched placebo tablets for both lemborexant 5 mg and lemborexant 10 mg. At the end of the study, participants will take lemborexant 5 mg one a day for a 1-week period prior to discontinuing drug.

Drug Titration. Since participants will be naïve to lemborexant, a 1-week period of lemborexant 5 mg once per day will be provided followed by titration to lemborexant 10 mg once per day; there will be matched placebo tablets for both lemborexant 5 mg and lemborexant 10 mg. At the end of the study, participants will be down-titrated to lemborexant 5 mg once a day for a 1-week period prior to discontinuing drug.

If the lemborexant dose is increased to 20 mg after the interim analysis, then the same up- and down-titration schedule will be followed except there will be an additional week at lemborexant 10 mg:
Up-titration: Lemborexant 5 mg—1 week
   Lemborexant 10 mg—1 week
   Lemborexant 20 mg—1 week
Down-titration: Lemborexant 10 mg—1 week
   Lemborexant 5 mg—1 week
   Off

Study outcomes. Measurements of primary outcomes, secondary outcomes, and exploratory outcomes are foreseen.

Primary outcomes: the primary outcome measure of the trial is amyloid PET SUVr at 6 months, measured and compared to placebo. Biomarker outcomes will include tau PET, measurements of Aβ, phospho-tau, and protofibrils in CSF and plasma.

Secondary outcomes: tau PET will be measured.

Exploratory outcomes: in CSF, the biomarkers Aβ, tau, phospho-tau, neurogranin (NG), neurofilament light chain (NfL) will be measured. In plasma, NfL, phospho-tau 181, and phospho-tau 217 will be measured. Clinical outcomes to measure cognition will include tests to obtain a Preclinical Alzheimer's Disease Cognitive Composite 5 (PACC5) scale for cognition and a Cognitive Functional Index (CFI).

ii. Preclinical Alzheimer's Disease—Elevated Levels of Amyloid

The goal of this trial is to determine if lemborexant treatment leads to primary prevention or delay of AD, through preventing early Aβ build-up in the brain. The trial will enroll cognitively-normal individuals with elevated levels of amyloid on screening PET imaging (approximately >40 centiloids), who are at high risk for cognitive decline over four years.

Patient Inclusion Criteria. Patients will be selected for intact cognition, determined by a Mini-Mental State Examination (MMSE) score of ≥27 after education adjustment, with a global clinical dementia rating (CDR) of zero. Patients will have elevated amyloid PET levels >40.

Lemborexant administration. Lemborexant or matched placebo will be administered to participants every month during in-person study visits where treatment adherence and adverse events will be assessed. Lemborexant is approved by the FDA for the treatment of insomnia in doses from 5-10 mg. After an interim analysis for safety and efficacy, the lemborexant dose may be increased to 20 mg. At the start of the study, participants will take lemborexant 5 mg once per day for a 1-week period and then increased to lemborexant 10 mg once per day; there will be matched placebo tablets for both lemborexant 5 mg and lemborexant 10 mg. At the end of the study, participants will take lemborexant 5 mg one a day for a 1-week period prior to discontinuing drug.

Drug titration. Since participants will be naïve to lemborexant, a 1-week period of lemborexant 5 mg once per day will be provided followed by titration to lemborexant 10 mg once per day; there will be matched placebo tablets for both lemborexant 5 mg and lemborexant 10 mg. At the end of the study, participants will be down-titrated to lemborexant 5 mg once a day for a 1-week period prior to discontinuing drug.

If the lemborexant dose is increased to 20 mg after the interim analysis, then the same up- and down-titration schedule will be followed except there will be an additional week at lemborexant 10 mg:
Up-titration: Lemborexant 5 mg—1 week
   Lemborexant 10 mg—1 week
   Lemborexant 20 mg—1 week
Down-titration: Lemborexant 10 mg—1 week
   Lemborexant 5 mg—1 week
   Off

Study outcomes. Measurements of primary outcomes, secondary outcomes, and exploratory outcomes are foreseen.

Primary outcomes. To test the effects of lemborexant on cognitive decline in patients with elevated amyloid, the primary outcome measure of the trial is the Preclinical AD Cognitive Composite 5 (PACC5) at 6 months. Biomarker outcomes will include tau PET, measurements of Aβ, phospho-tau, and protofibrils in CSF and plasma.

Second outcomes. The Cognitive Function Index (CFI) will be measured. Amyloid PET and tau PET will be measured.

Exploratory outcomes. Clinical measurements will be ADCS ADL-prevention, Computerized Cognitive Composite, ISLT, Trails, CDR-SB, and time to CDR 0.5. vMRI and rs-MRI will be determined. In CSF, the biomarkers Aβ, tau, phospho-tau, neurogranin (NG), neurofilament light chain (NfL) will be measured. In plasma, NfL, phospho-tau 181, and phospho-tau 217 will be measured.

c. Trial 3: Treatment of Subjects Having Preclinical or Early Alzheimer's Disease

This trial will evaluate the efficacy of lemborexant in the prevention or delay of Aβ accumulation, spread of downstream tau pathology, and cognitive decline across the continuum of preclinical and early AD. This trial will follow the protocol of Trial 2, with the exception that different dosages will be used. Instead of 5 mg of lemborexant, 7.5 mg of lemborexant will be used. Instead of 10 mg lemborexant, 15 mg of lemborexant will be used. Instead of 20 mg of lemborexant, 25 mg of lemborexant will be used or 30 mg of lemborexant will be used.

### Example 2: Mouse Study of Tau-Mediated Neurodegeneration

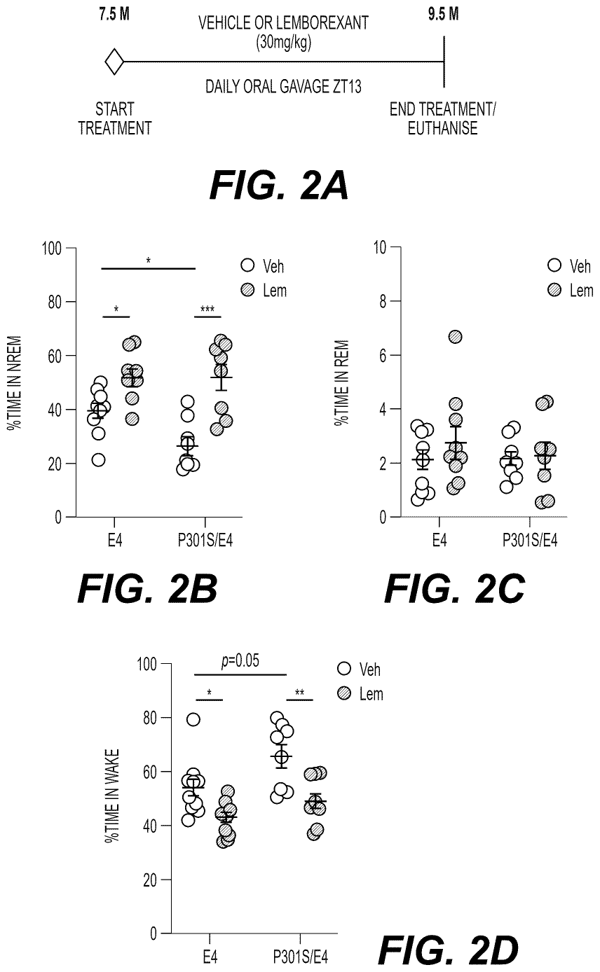

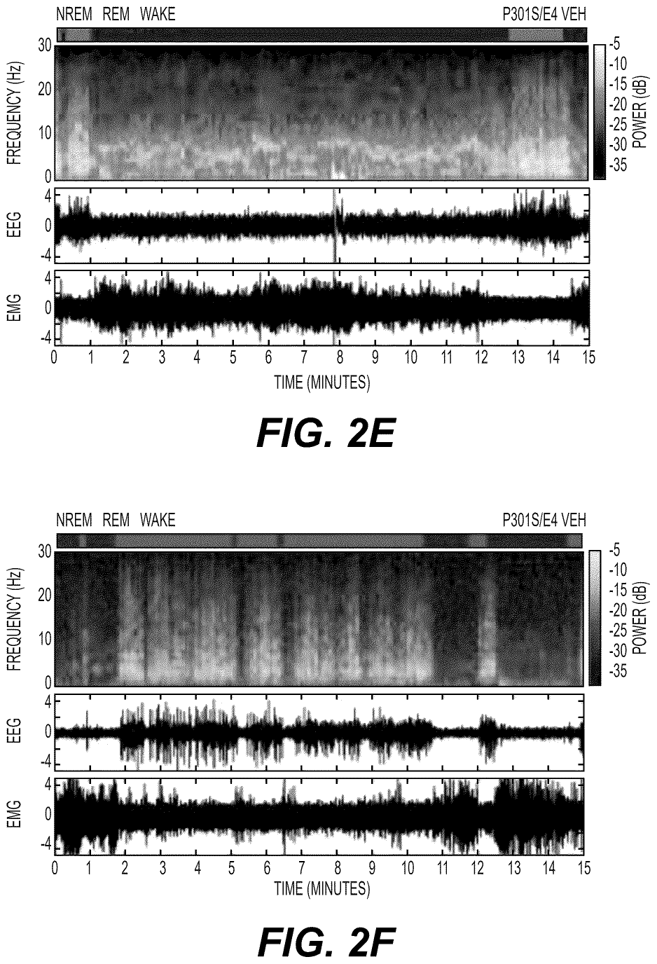

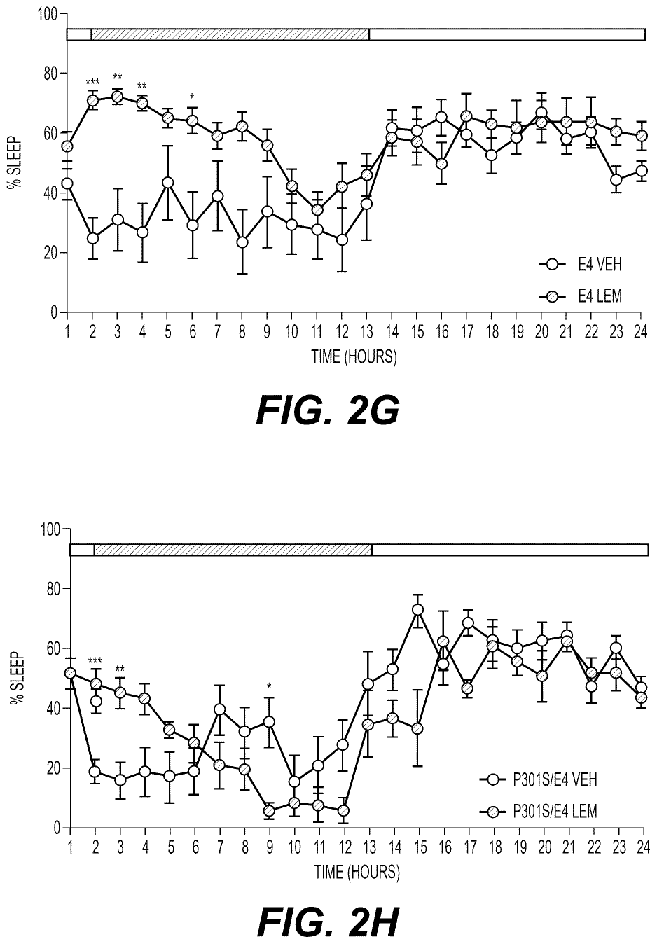

P301S/E4 and E4 knock-in non-tau depositing mice were orally gavaged with 30 mg/kg lemborexant or vehicle daily from 7.5 months of age, when tau-mediated neuroinflammation is observed without overt neuronal loss, until 9.5 months of age (FIG. 2a).

### A. Changes in Sleep-Wake Behavior

Changes in sleep-wake behavior were validated by electroencephalography (EEG) analyses. Lemborexant-treated E4 and P301S/E4 mice showed approximately 25% increased time spent in NREM sleep as well as approximately 20% reduction in time spent awake (FIG. 2B-FIG. 2F). No changes in REM sleep time were observed consistent with previous findings.

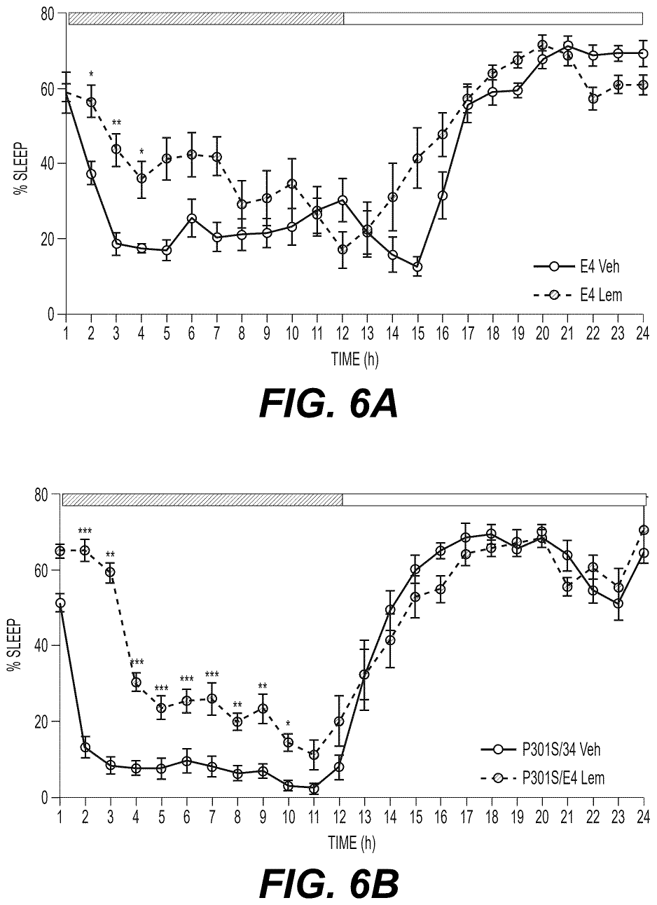

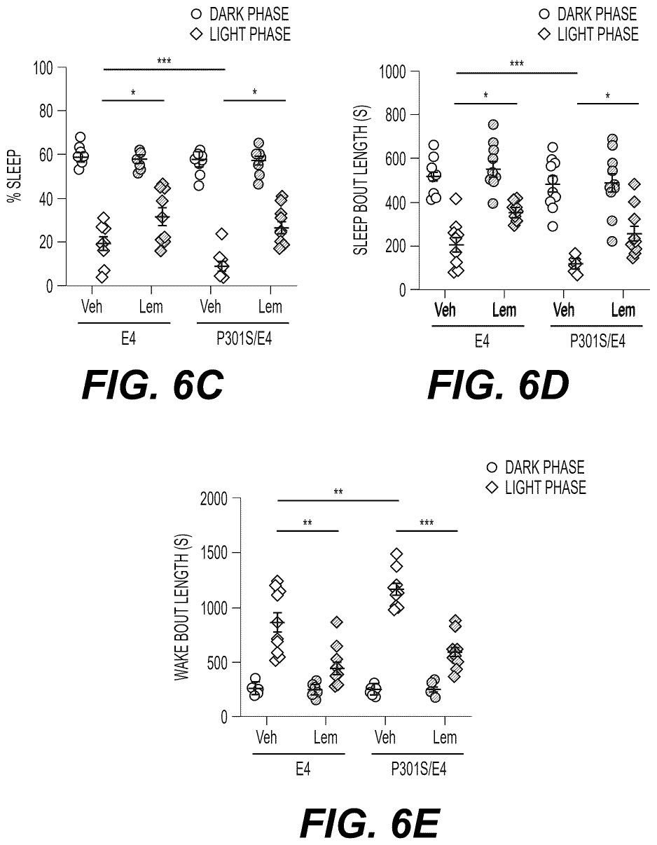

Tau-dependent reduction in NREM sleep was observed in P301S/E4 mice compared to E4 mice (FIG. 2B), indicating that pathological tau is linked with sleep. The effects of lemborexant on sleep (FIG. 2G and FIG. 2H) and sleep-associated locomotor activity (FIG. 6), measured by EEG and Piezosleep pads, lasted approximately five hours post-treatment with no additional phase delays or changes in circadian sleep-wake activity.

### B. Effect on Tau Pathology and Neurodegeneration

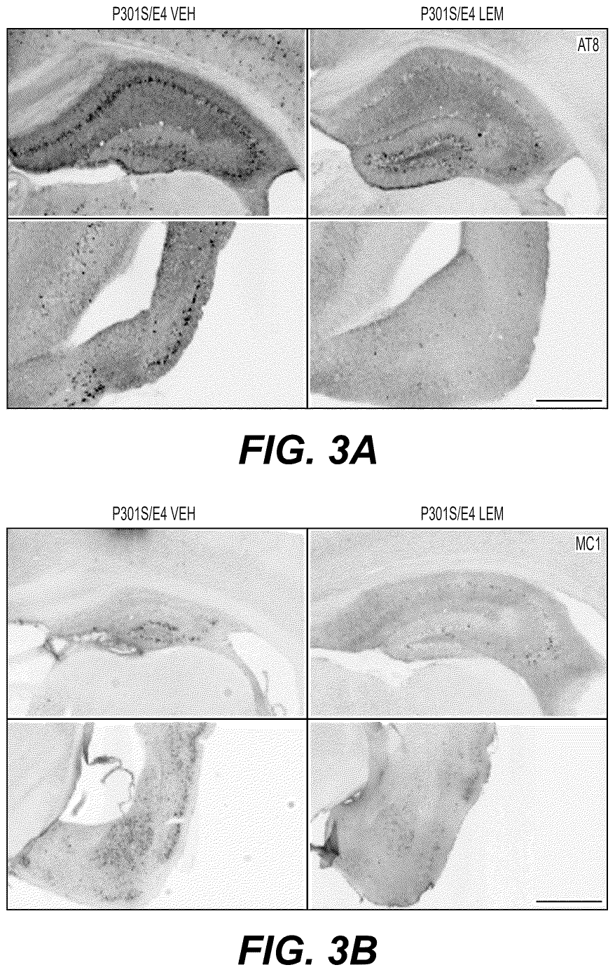

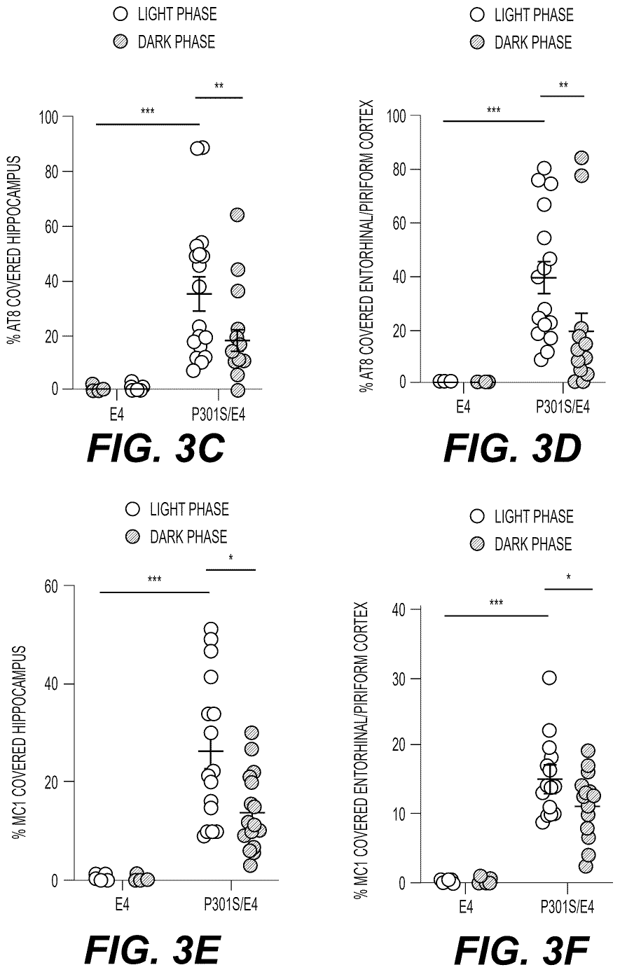

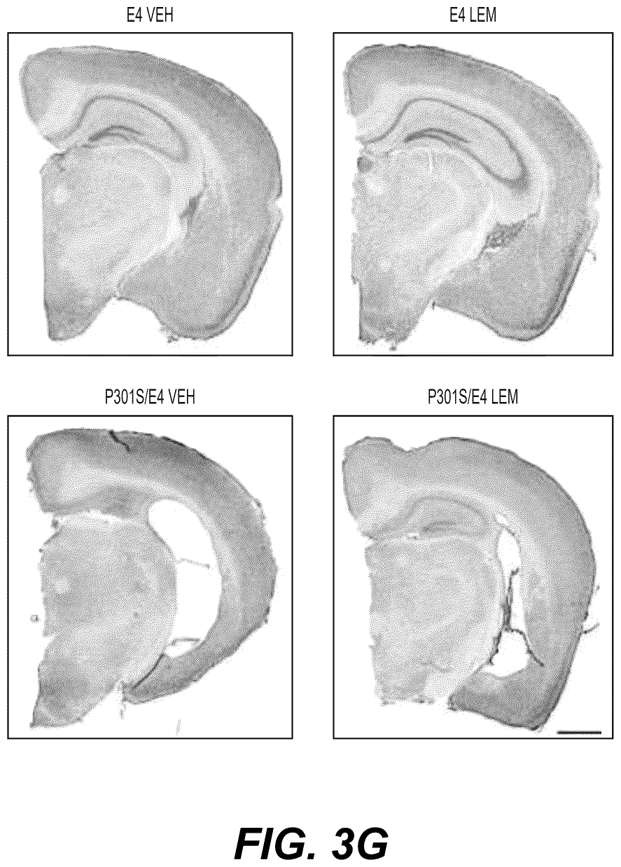

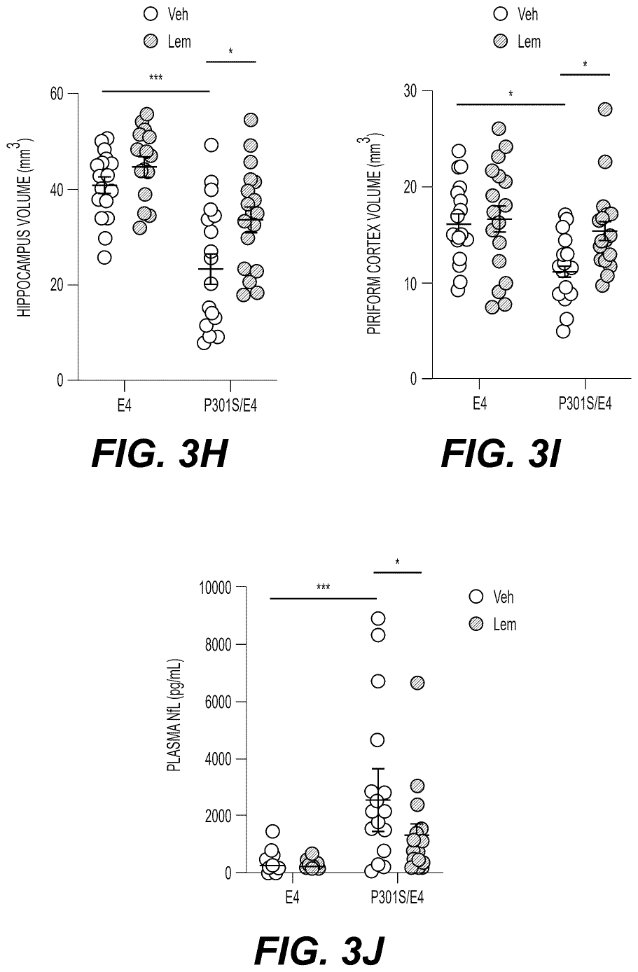

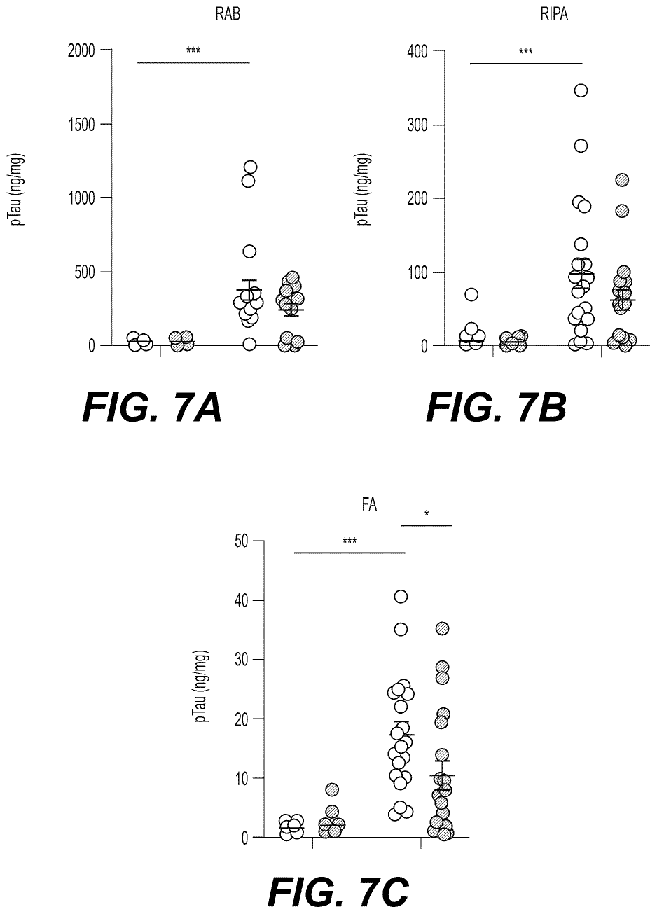

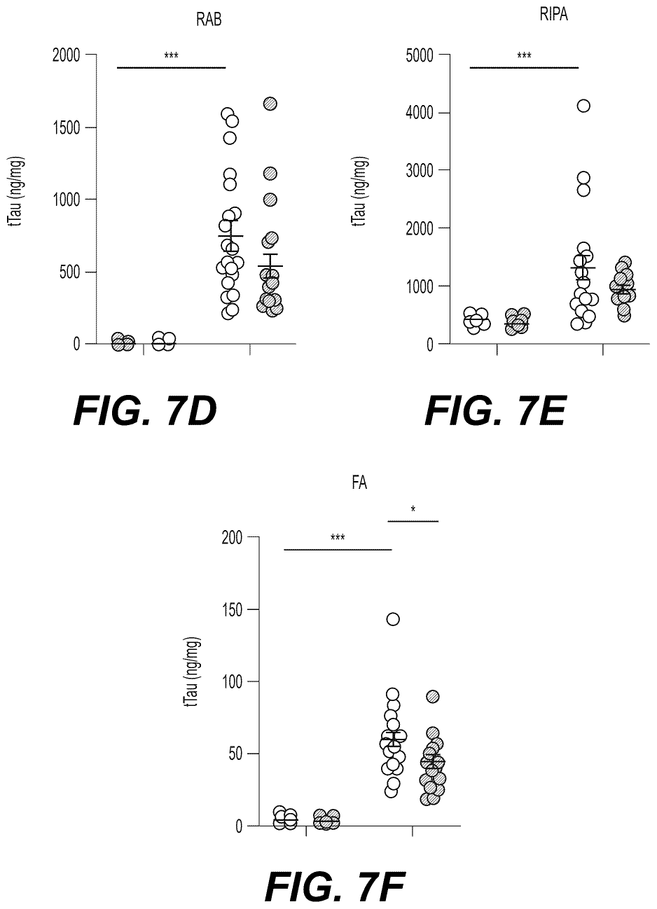

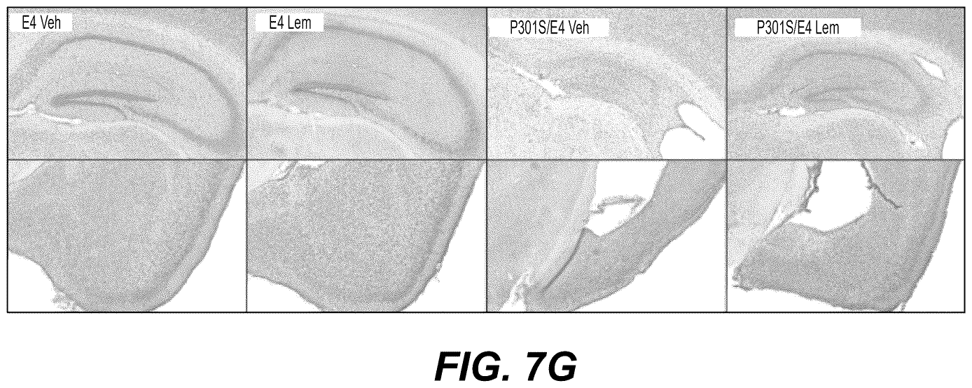

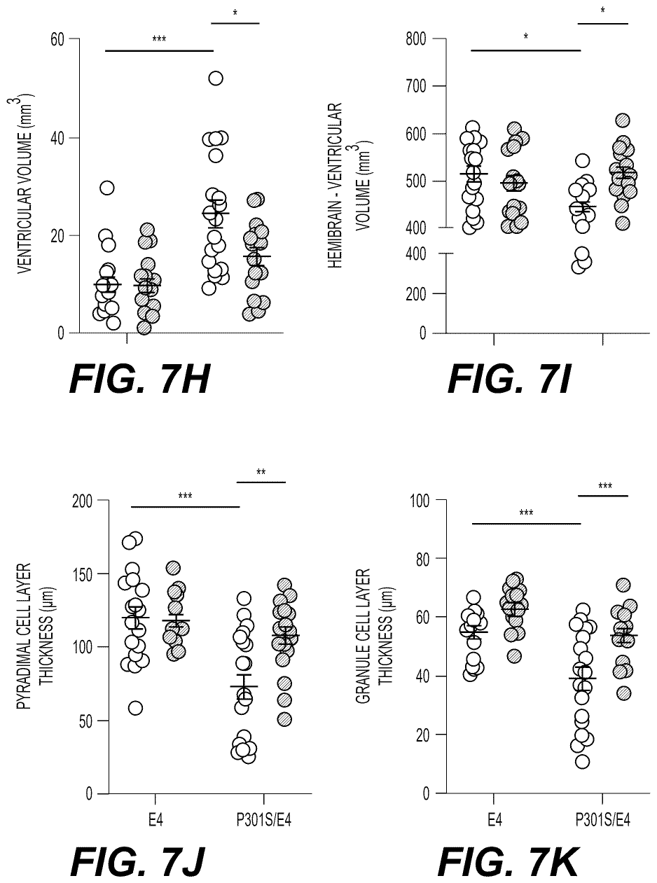

To determine whether lemborexant could impact tau pathology and neurodegeneration, the hippocampus and entorhinal and piriform cortices, which are regions that display substantial tau-mediated degeneration, were investigated. A significant reduction by approximately 20% in AT8+ and MC1+ tau staining controls was observed in lemborexant-treated P301S/E4 mice compared to vehicle-treated (FIG. 3A-FIG. 3F). Additionally, lemborexant-treated P301S/E4 mice had significantly decreased insoluble phosphorylated and total tau levels (FIG. 7A-FIG. 7F).

The extent of brain atrophy in lemborexant-treated P301S/E4 mice was found to be strongly attenuated compared to controls (FIG. 3G-FIG. 3I and FIG. 7H-7K). More specifically, atrophy of the hippocampus and piriform/entorhinal cortex was significantly ameliorated by approximately 50% concomitant with less enlargements of the lateral ventricles. The granule cell and pyramidal cell neuronal layers were visibly thicker in lemborexant-treated P301S/E4 mice (FIG. 7G-FIG. 7K), which was validated by reduced plasma neurofilament light chain levels (FIG. 3J), evidencing a robust improvement in neuronal damage and degeneration.

### C. Microglial Reactivity

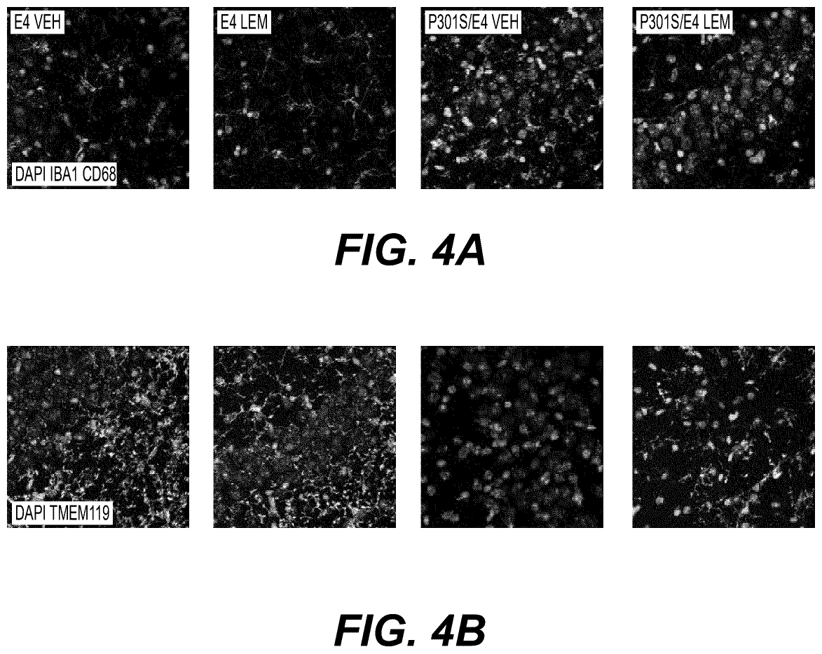

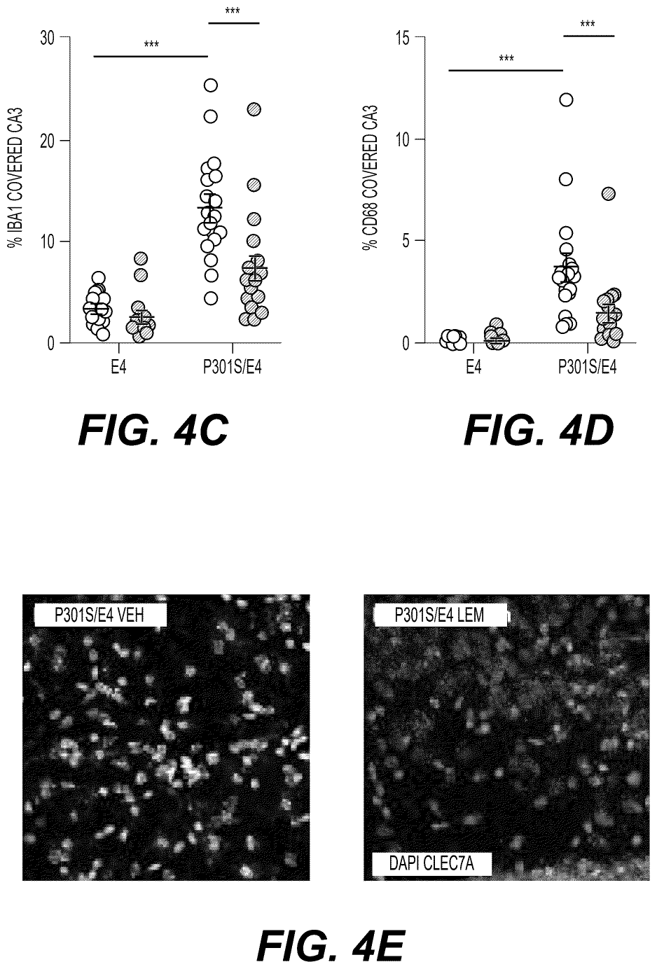

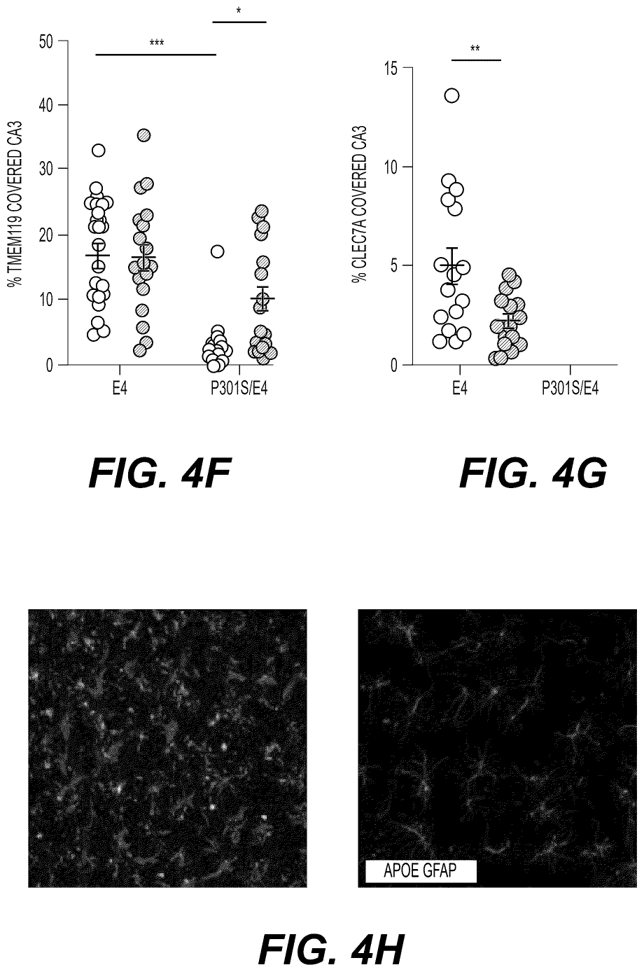

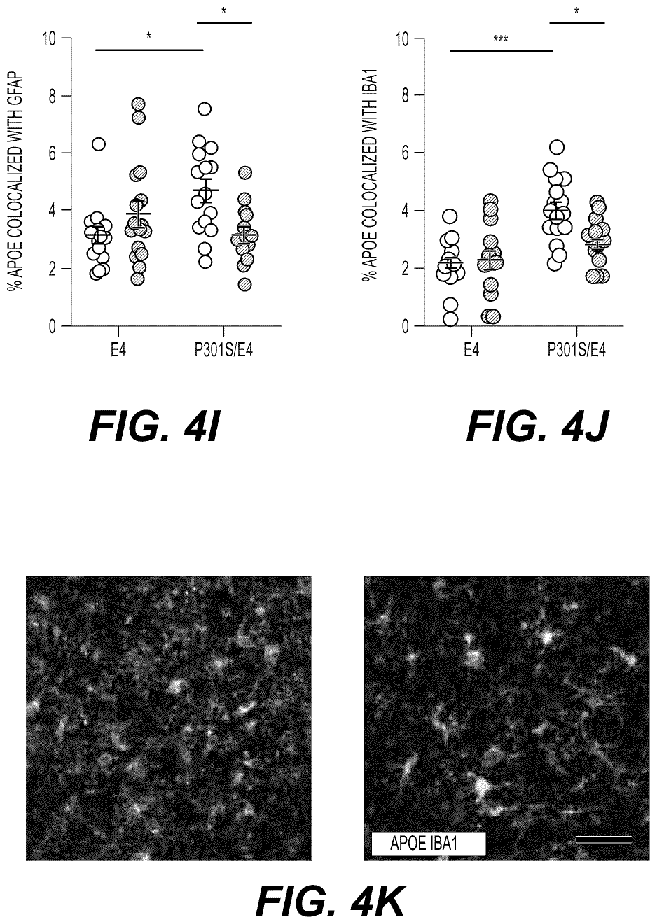

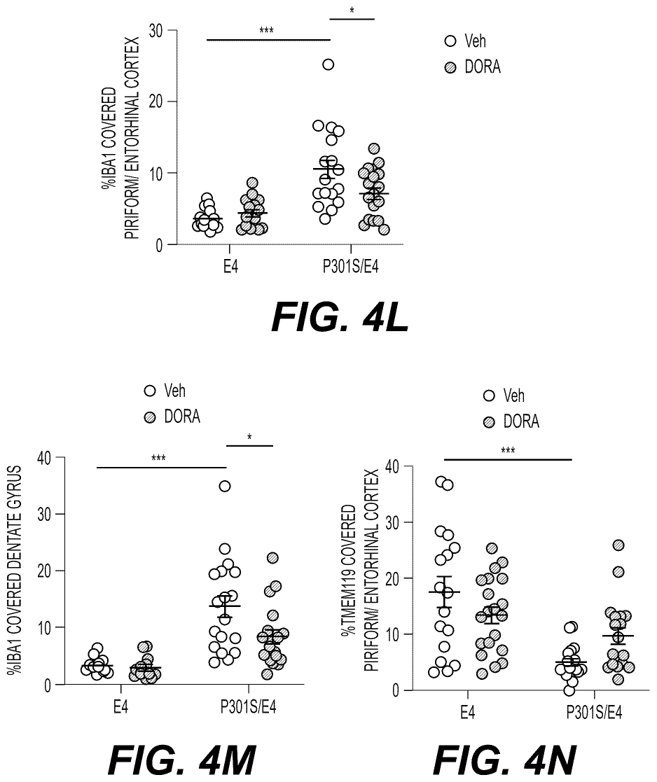

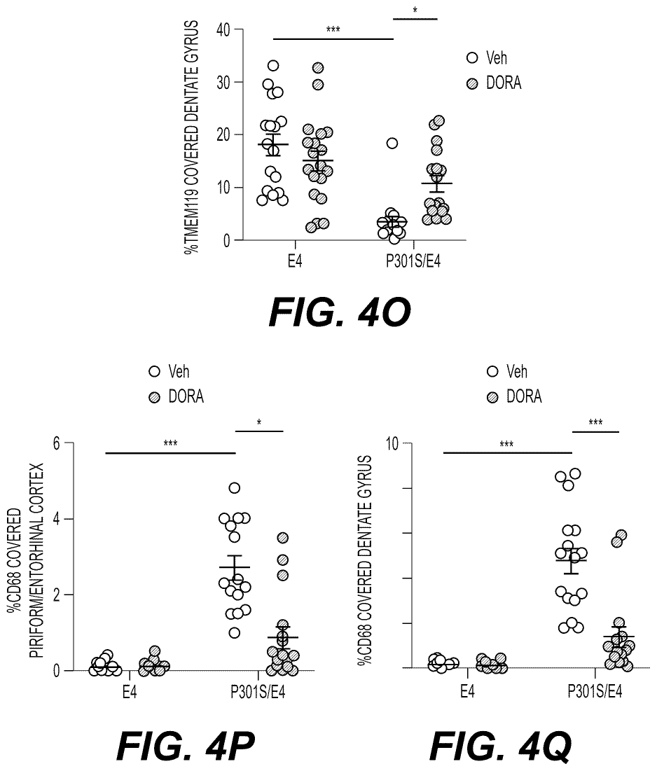

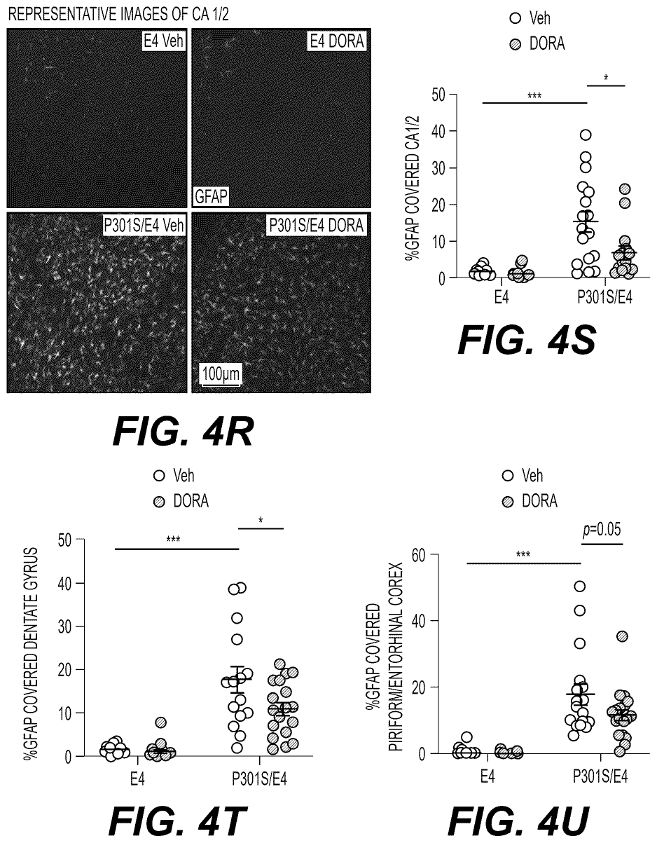

To investigate whether the marked reduction in tau-mediated neurodegeneration in lemborexant-treated mice was linked with reduced microglial reactivity, different markers of reactive microglia across the spectrum of disease-associated or homeostatic populations in both hippocampus and piriform cortex were quantified. Substantial changes, predominantly in the hippocampus and most notably in the CA3 regions (FIG. 4), were observed (FIG. 4).

A marked reduction in ionized calcium-binding adaptor molecule 1 (IBA1) was observed, indicating an overall reduction in reactive microglial population in lemborexant-treated P301S/E4 mice compared to vehicle-treated P301S/E4 mice (FIG. 4A, FIG. 4C, FIG. 4L, and FIG. 4M).

Disease-associated microglial markers such as Clec7a (FIG. 4E and FIG. 4G) and CD68 (FIG. 4A, FIG. 4D, FIG. 4P, and FIG. 4Q), a marker of phagolysosomal activity, were substantially increased in P301S/E4 vs. E4 mice, though lemborexant strongly reduced these markers relative to P301S/E4 treated with vehicle.

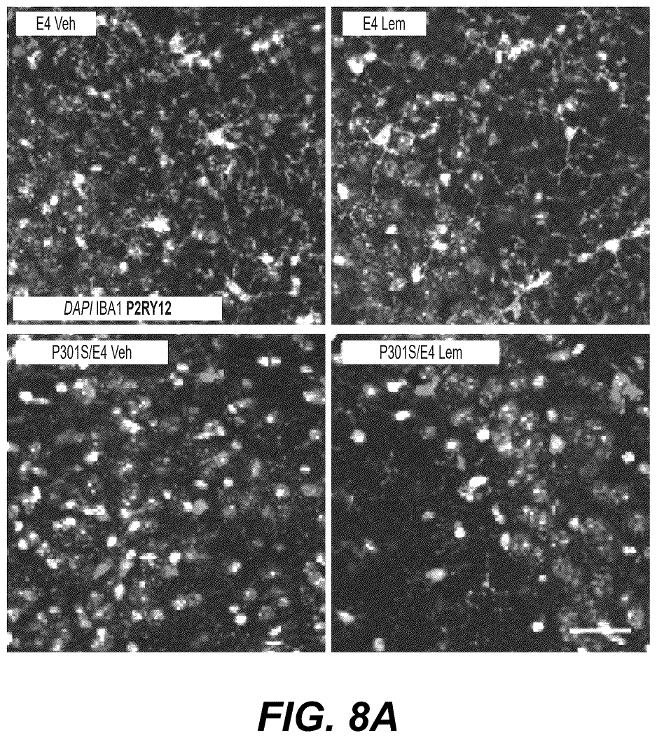

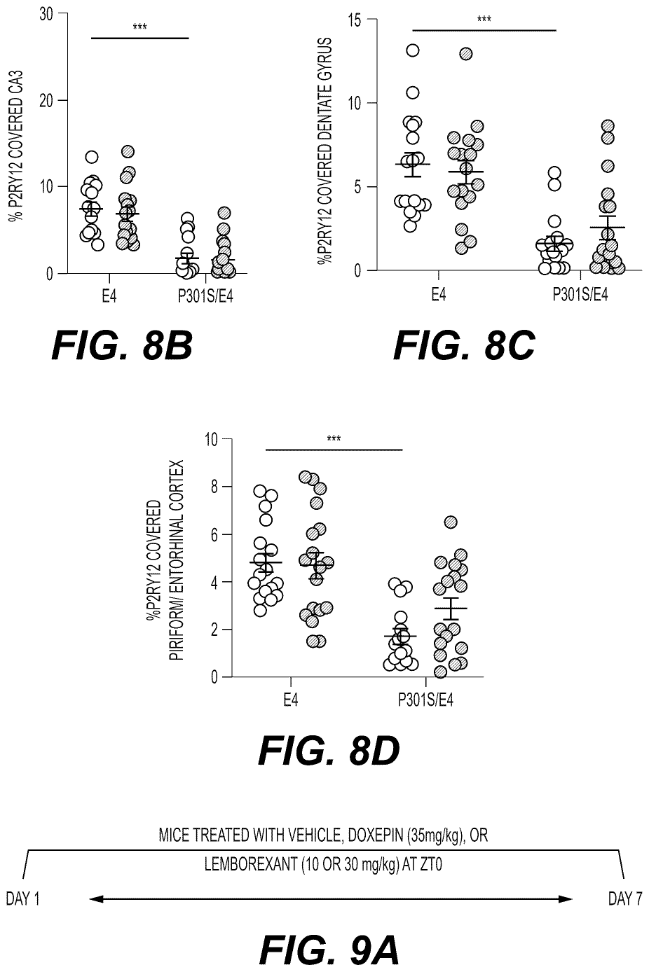

A significant increase in TMEM119 marker of homeostatic microglia in P301S/E4 mice-treated with lemborexant was observed (FIG. 4B, FIG. 4F, FIG. 4N, and FIG. 4O), with no changes observed in P2RY12 (FIG. 8).

These results suggest that decreasing reactive microglia are involved in the effect of lemborexant in modulating tau-mediated neurodegeneration. Moreover, both astroglial and microglial APOE co-localization were significantly reduced in lemborexant-treated P301S/E4 mice compared to controls (FIG. 4H-FIG. 4K), the latter of which is more commonly observed during elevated inflammatory and damaging conditions only. Lemborexant-treatment reduced GFAP+ astroglial reactivity in P301S/E4 mouse hippocampus (FIG. 4R-FIG. 4T). No changes in microglial reactivity in the E4 knock-in mice in the absence of tau pathology were found.

### D. RNA Sequencing

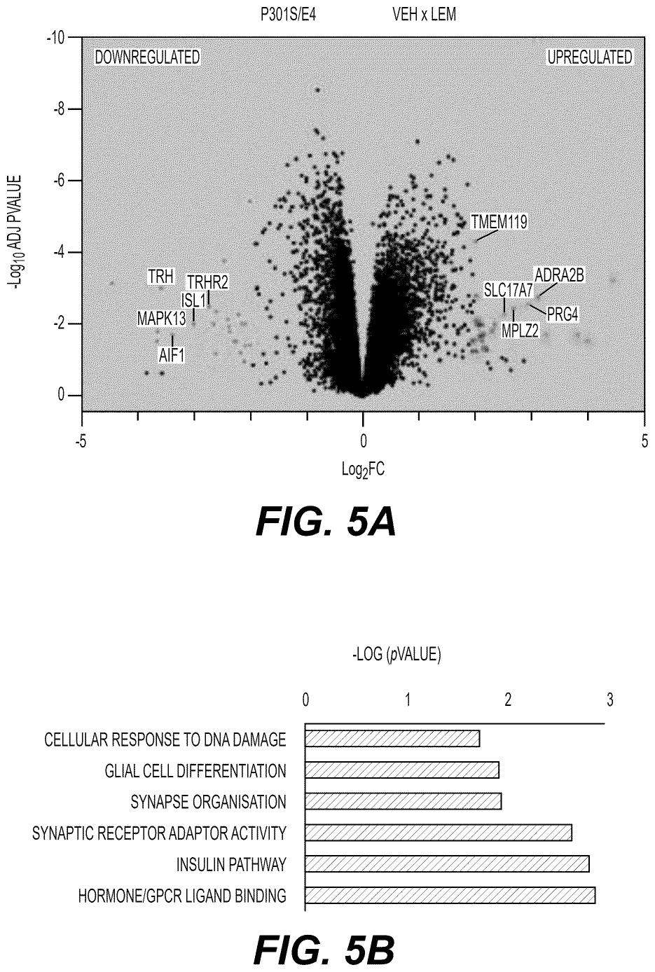

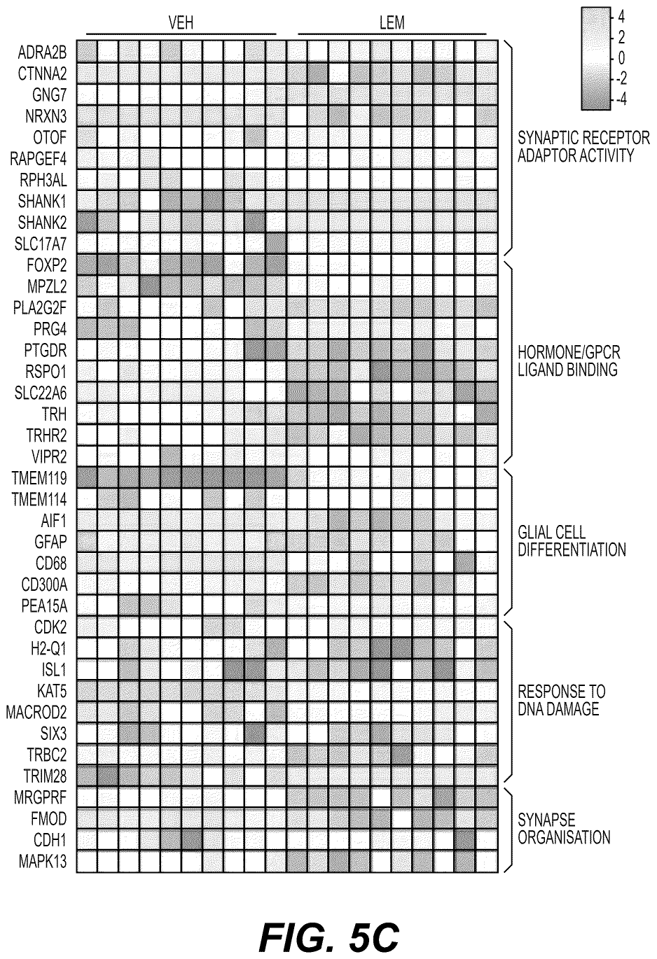

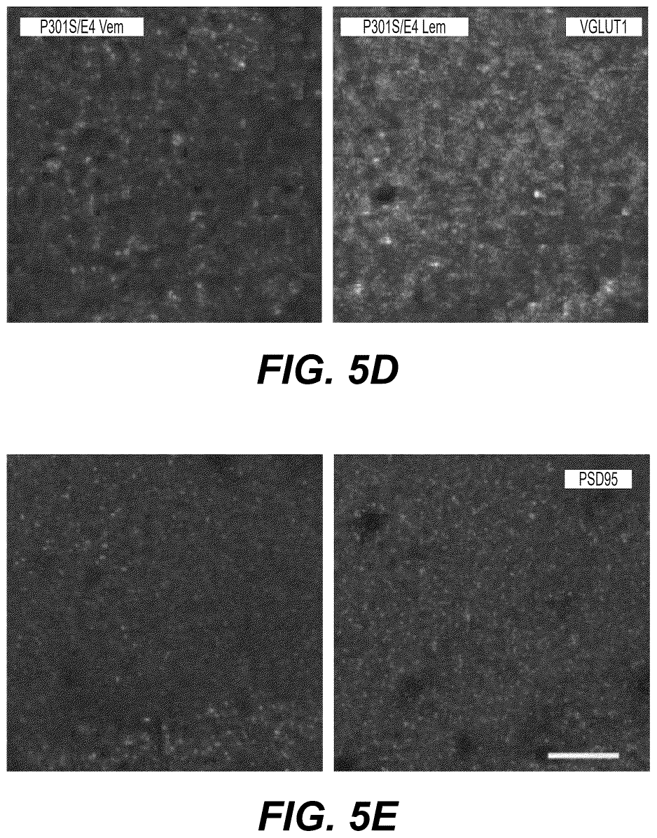

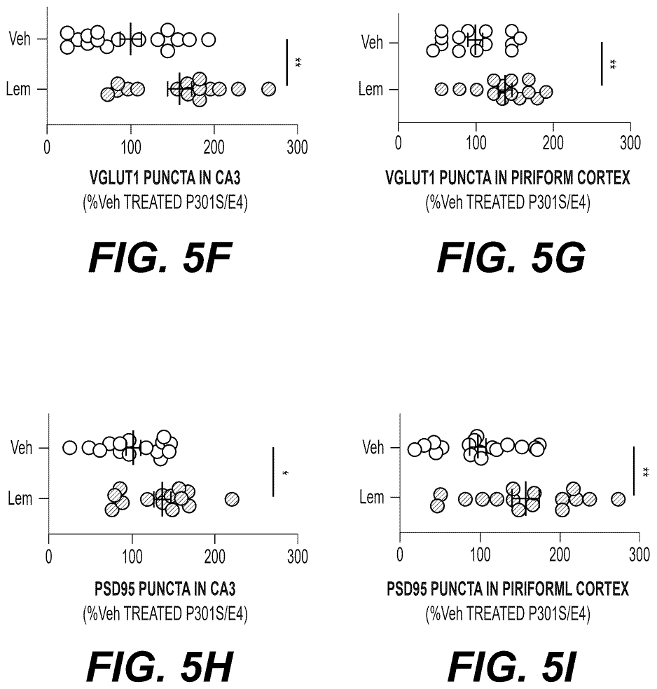

To gain insights into the mechanisms behind the observed changes in microglial reactivity and tau-mediated neurodegeneration by lemborexant-induced NREM sleep, RNA sequencing in bulk hippocampal tissue was performed. Gene expression changes were found and were involved in multiple functional modules including hormonal and GPCR ligand binding, glial cell differentiation, synaptic modulation, particularly of excitatory synapses as well as response to DNA damage (FIG. 5). In addition to normal aging, defective DNA repair has also been linked to age-associated neurodegenerative diseases such as AD. Hyperphosphorylated and aggregated tau may hinder DNA repair by interacting with DNA repair proteins.

Based on these data, sleep in which there is overall decreased neuronal metabolism and possibly synaptic activity could play a key role in neuronal genome maintenance and DNA repair function. Nonetheless, further research is needed to understand a link between sleep, DNA damage and neurodegeneration. Interestingly, genes such as Adra2b, Trh, Trhr2, Mpzl2, Slc22a6, Pla2g2f, Ptgdr, Foxp2 are genes that modulate sleep. More specifically, thyrotropin-releasing hormone (Trh) and its receptor (Trhr2) modulate behavioral arousal, in part, through orexin. TRH application transforms GABAergic neurons from the burst-firing mode typically associated with the synchronized cortical activity that occurs during NREM sleep to the tonic, single-spike mode of action potential generation associated with desynchronized cortical activity that occurs during wakefulness and REM sleep. A downregulation of Trh and Trhr2 suggests that DORA-induced NREM sleep further interacts with humoral regulation of sleep-wake behavior to promote sleep, particularly in the presence of tau, as these effects were absent in non-tau E4 mice. In support of these findings, we observed changes in Slc22a6, Pla2g2f, Ptgdr expression that modulate sleep-wake through potent endogenous somnogens such as prostaglandins. In addition to catalyzing biosynthesis of prostaglandins, phospholipase A2 play a major role in cell growth differentiation and inflammation, and are associated with metabolic changes in patients with obstructive sleep apnea. Comparably, genes involved in glial cell differentiation including microglia expressed Tmem119, Tmem114, Cd68, Aif1, Cd300a, Pea15a and H2-Q1 were differentially regulated in lemborexant-treated P301S/E4 mice (FIG. 5C), suggesting that promoting lemborexant-mediated NREM sleep or inhibiting orexin signaling influences important immune functions such as T-cell antigen representation, response to injury and regulation of apoptosis, all of which may directly modify tau-mediated neurodegeneration. Further supporting this principle is the decrease in pre-synaptic vesicular glutamate transporter (VGLUT1, Slc17a7) and postsynaptic density markers (PSD95, Shank1, Shank2), both transcriptionally and immunohistochemically (FIG. 5C-FIG. 5I). Transcriptional changes in synaptic receptor activity and organization such as Otof, Nrxn3, Mrgprf, Mapk13 and Fmod among others (FIG. 5C), confirm that promoting NREM sleep or inhibiting orexin receptor signaling decreases tau-mediated neurodegeneration as well as associated synaptic loss.

### E. Methods and Analyses

Mice: All animal procedures and protocols were approved by the Animal Studies Committee at Washington University School of Medicine. PS19 tau transgenic mice harboring 1N4R tau and overexpressing human P301S tau mutation were used24. These mice have been backcrossed to C57BL/6 for more than ten generations. Human apoE4 knock-in mice were generated as described in Mol. Neurodegener. 14, (2019) and crossed to P301S mice for several generations to produce experimental P301S/E4 mice. Littermates of same sex were randomly assigned to experimental groups. Only male animals were used and sacrificed at 9.5 months of age. All mice were housed in specific pathogen-free conditions and under the same 12 h light/dark cycle, ambient room temperature as well as with food and water available ad libitum.

Treatment: Mice were gavaged daily with a single 30 mg/kg dose of lemborexant or 0.5% methylcellulose vehicle at ZT13, one hour post dark onset, starting at 7.5M of age until euthanizing at 9.5M of age.

Tissue collection: All mice were perfused between ZT3 and ZT7, at a time window when mice were sleep deprived, to avoid circadian influence on transcriptional fluctuation of microglial gene expression. Prior to transcardiac perfusion, mice were anesthetized with pentobarbital (50 mg/kg, intraperitoneal). Blood was collected from the heart before transcardiac perfusion, which was centrifuged at 5000×g for 5 mins at 4 °C to obtain plasma. Mice were transcardially perfused with ice-cold phosphate-buffered saline containing 0.3% heparin. One hemibrain was dissected, snap-frozen and stored at −80 °C for biochemical analyses. The other hemibrain was immerse-fixed for 24 h in 4% paraformaldehyde following by cryoprotection in 30% sucrose for 48 h and frozen at −80 °C until tissue samples were sectioned for immunohistochemical analyses.

Measurement and analysis of sleep-wake states: Sleep-wake behavior in mice were monitored using electroencephalography (EEG) and independently using PiezoSleep mouse behavioral tracking system (SignalSolutions).

For EEG experiments, animals were anesthetized with isofluorane (0.5-3%). Any signs of pain were assessed by toe pinching before an incision was made. Mice were then surgically implanted with screw electrodes in the skull for EEG and stainless wire electrodes in the nuchal muscle for electromyography (EMG). After a midline vertical incision to expose the skull, forceps and 3% hydrogen peroxide were used to remove any connective tissue and dry the skull for electrode placement. Burr holes for the frontal reference electrodes were made (anterior +0.5 mm, lateral ±0.5 mm; bregma) using a micro drill with a 0.9 mm tip and screws were secured in the skull. Two bilateral active recording electrodes were placed over the parietal cortex (posterior −2.5 mm, lateral 1.5; bregma) and a ground screw secured over the cerebellum (posterior −6.2 mm, lateral ±05; bregma), using the same technique as the reference electrode. The exposed skull, screws, and all wires were covered in a layer of dental cement (SNAβ, Parkell) with the pin header secured to the head for subsequent recording. The skin was sutured around the exposed dental cement/pin header and tissue glue (Vetbond, 3M) used to close the remainder of the incision. After the procedure, mice were placed in a warmed chamber to fully recover from anesthesia and individually housed in monitoring cages with fresh bedding, water, food and Carprofen (orally; % tab of 5 g tab; ad libitum) supplement. After recovery from surgery over three days, mice were habituated in the recording cage for two weeks, followed by undisturbed EEG/EMG recording performed for two consecutive days in freely moving mice. Bilateral cortical EEG signals were acquired by a P511K A.C. Preamplifier (Grass-Telefactor Instruments, Warwick, RI USA), digitized with a BIOPAC MP150, recorded digitally using the BIOPAC's AcqKnowlege software with a sampling rate of 250 Hz, and converted into (.edf) format for analysis. EEG was processed in MATLAB (MathWorks) through a band-pass filter of 1-30 Hz to remove DC offset and high-frequency noise. EEG/EMG recordings were manually scored in 10-second epochs for wake, NREM and REM sleep to create a calibration file containing mixture-z scoring variables specific for the recording subject. The calibration file was imported into AccuSleep, a machine learning-based, automated sleep scoring program in MATLAB, to complete the remainder of the scoring.

PiezoSleep mouse behavioral tracking system (Signal Solutions, LLC, Lexington, KY, USA) was used. The non-invasive method includes a thin dielectric piezo sensor pad that generates a voltage signal in response to changes in real-time fluctuations in pressure on its surface. Mice were individually housed with the piezo pad underneath fresh bedding, with fresh water and food available ad libitum and recorded without disturbance over a period of six days. Data was acquired using SleepStats software (Signal Solutions, LLC, Lexington, KY, USA).

Volumetric analysis: Volumetric analysis of the hippocampus, entorhinal/piriform cortex, and ventricle was performed via stereological methods by assessing sections spaced by 180 μm starting from bregma −1.3 mm to bregma −3.1 mm (16-18 sections per mouse depending on the severity of brain atrophy). 30 μm microtome-cut sections mounted on slides were briefly immersed in distilled water before incubating in pre-warmed 0.1% cresyl violet at 37 °C for six minutes. Following this, tissues were rinsed in distilled water and transferred to 70%, 95% and 100% ethanol sequentially, for two minutes each. Slides were then cleared in xylene before finally coverslipping with cytoseal60 mounting medium (Thermo Fisher Scientific). Slides were scanned using Hamamatsu's Nanozoomer microscope at 20× magnification. Hippocampus, EC/PC, and ventricles were traced using NDP view 2. The formula for the volumetric calculation was volume=(sum of area)*0.3 mm.

Neuronal layer thickness measurement: Dentate granular and entorhinal pyramidal cell layers were measured across three sections by drawing a scale line crossed the cell layer using NDP.view 2 and calculating the average value for each mouse.

Immunohistochemistry: Free-floating sections were briefly washed with TRIS buffered saline with 1% TritonX100 (TBS-Tx) before quenching endogenous peroxidase with 0.3% hydrogen peroxide for 20 mins at room temperature. After a brief wash, sections were blocked using 5% goat serum for 30 mins at room temperature, followed by primary antibody incubation in biotinylated AT8 (Phospho-Tau Ser202, Thr205; 1:500, MN1020B, Thermo Fisher Scientific) or MC1 (1:500, kindly gifted by Dr Peter Davies), overnight at 4 °C. The following day, MC1 stained sections were briefly washed and incubated in HRP-conjugated secondary antibody for one hour at room temperature. Both AT8 and MC1 stained tissue were then developed with 3,3′-diaminobenzidine (DAB, Sigma) for 10 minutes and 14 minutes, respectively. Tissue sections were mounted on slides and dehydrated using a series of increasingly concentrated ethanol before finally immersing in xylene and coverslipping using Cytoseal mounting medium. Slides were scanned using Hamamatsu's Nanozoomer microscope at 20× magnification.

Immunofluorescence stainings: Free-floating sections were washed briefly with PBS and blocked in 5% donkey serum for 1 h at room temperature. Primary antibodies were diluted in blocking buffer and incubated at 4 °C overnight with slow agitation unless stated otherwise. Primary antibodies were used as follows: IBA1 (1:500; 019-19741, Fujifilm or NB100-1028, Novus Biologicals), CD68 (1:00; FA-11, BioRad), P2RY12 (1:100 at room temperature, HPA013796, Sigma-Aldrich), TMEM119 (1:500; E3E10, Cell Signaling Technology), Clec7a (1:50 at room temperature; mabg-mdect, InvivoGen), GFAP (1:2000; 2E1.E9 Alexa Flour 488-conjugated, BioLegend), APOE (1:300; D719N, Cell Signalling), PSD-95 (1:200, 51-6900, Thermo Fisher Scientific), VGLUT1 (1:200, AB5905, Merck Millipore). The next day, sections were washed and incubated with secondary antibody diluted in blocking buffer, followed by a 4′,6-Diamidin-2-phenylindol (DAPI, 5 μg/mL) where appropriate before mounting sections onto slides (Prolong™ Gold Antifade reagent, Thermo Fisher Scientific).

Confocal imaging and analyses: Images were acquired using a Leica Stellaris 5 confocal microscope and the Leica Application Suite X software (4.2.1.23810). Laser and detector settings were maintained constant for the acquisition of each immunostaining. For all analyses, at least two images were taken per brain region and slide using 20× (Apo CS 10×/0.40 dry), 40× (Apo CS 40.0×1.25) and 63× (Apo CS 63.0×1.4 Oil) differential interference contrast objective, respectively, at 1024×1024 pixel resolution, with a 15 μm z-step thickness. For imaging synapses, Leica Stellaris 8 Lightning was used to produce adaptive deconvolution based super-resolved confocal images using 63× oil objective. Image analyses was performed using Fiji (ImageJ). For the feasibility of the quantification, all layers from a single image stack were projected on a single slice (Stack\Z projection). Next, microglia were segmented using automatic thresholding methods in Fiji and presented as % area covered by selected stain in hippocampus or entorhinal/piriform cortex.

Protein extraction: Frozen mouse hippocampal tissue was weighed and homogenized in a bullet blender homogenizer (Next Advance) using beaded tubes with 200 μl RAB buffer pH 7.0 (100 mM MES, 1 mM EGTA, 0.5 mM MgSO4, 750 mM NaCl, 20 mM NaF, 1 mM Na3VO4) supplemented with 1× protease inhibitor (cOmplete™, Roche) and 1× phosphatase inhibitor (PhosSTOP, Roche). This homogenate was centrifuged for five min at 4 °C at 5000×g to pellet RAB insoluble material and the supernatant was ultracentrifuged for 20 minutes at 50,000×g with an MLA-130 rotor in an Optima MAX-XP ultracentrifuge (Beckman Coulter) to obtain the RAB extracts. From the remaining cellular pellet, proteins were extracted with RIPA buffer pH 8.0 (150 mM NaCl, 50 mM TRIS, 0.5% deoxycholic acid, 1% Triton-X 100, 0.1% sodium deoxycholate, 5 mM EDTA, 20 mM NaF, 1 mM Na3VO4) supplemented with protease and phosphatase inhibitors. After a five min clearing for RIPA insoluble material at 5000×g at 4 °C, the supernatant was again ultracentrifuged for 30 minutes at 50,000×g to obtain the RIPA soluble protein fraction. The RIPA insoluble pellet was dissolved with ice-cold 70% formic acid (FA) and sonicated for one minute at 30% amplitude in short pulses at room temperature using sonicator (Model FB120, Fisher Scientific), followed by a final ultracentrifugation for 20 minutes at 50,000×g at 4 °C. Protein concentrations were measured for RIPA fractions using a BCA assay (Pierce). All samples were aliquoted and frozen at −80 °C until use.

Tau ELISA: Human tau and pTau were measured in in RAB, RIPA and 70% FA fractions using a sandwich ELISA and normalized to tissue weight as described28. The coating antibodies for total human tau, and pTau were TAU-5 (mouse monoclonal, 20 μg/ml), and HJ14.5 (mouse monoclonal, 20 μg/ml), respectively. The capture antibodies for total human tau and pTau were HT7-biotinlyated (MN1000B, ThermoFisher Scientific) and AT8-biotinlyated (MN1020B, thermos Fisher Scientific), respectively.

NFL concentration: Plasma NFL concentration was measured with NF-Light Simoa Assay Advantage kit using Quanterix. The measurement was performed following the manufacturer's instructions.

RNA extraction: Frozen hippocampal tissue was weighed and homogenized in RNAase-free beaded tubes (REDE, Next Advance) in chloroform with TRIzol™. Samples were centrifuged for 15 minutes at 12,000×g at 4 °C and the aqueous upper supernatant transferred for RNA isolation with the RNeasy Mini Kit (Qiagen) following manufacturer's instructions. RNA quality was controlled using Bioanalyzer prior to Next Generation Sequencing by Clontech SMARTer.

RNA sequencing and analyses: Samples were prepared according to library kit manufacturer's protocol, indexed, pooled, and sequenced on an Illumina NovaSeq 6000. Basecalls and demultiplexing were performed with Illumina's bcl2fastq software and a custom python demultiplexing program with a maximum of one mismatch in the indexing read. RNA-seq reads were then aligned to the Ensembl release 76 primary assembly with STAR version 2.7.9a (Doblin et al). Gene counts were derived from the number of uniquely aligned unambiguous reads by Subread:featureCount version 2.0.3. Isoform expression of known Ensembl transcripts were quantified with Salmon version 1.5.2. Sequencing performance was assessed for the total number of aligned reads, total number of uniquely aligned reads, and features detected. The ribosomal fraction, known junction saturation, and read distribution over known gene models were quantified with RSeQC version 4.0. All gene counts were then imported into the R/Bioconductor package EdgeR and TMM normalization size factors were calculated to adjust for samples for differences in library size. Ribosomal genes and genes not expressed in the smallest group size minus one sample greater than one count-per-million were excluded from further analysis. The TMM size factors and the matrix of counts were then imported into the R/Bioconductor package Limma. Weighted likelihoods based on the observed mean-variance relationship of every gene and sample were then calculated for all samples with the voomWithQualityWeights function and were fitted using a Limma generalized linear model with additional unknown latent effects as determined by surrogate variable analysis (SVA). The performance of all genes was assessed with plots of the residual standard deviation of every gene to their average log-count with a robustly fitted trend line of the residuals. Differential expression analysis was then performed to analyze for differences between conditions and the results were filtered for only those genes with Benjamini-Hochberg false-discovery rate adjusted p-values less than or equal to 0.05.

For each contrast extracted with Limma, global perturbations in known Gene Ontology (GO) terms, MSigDb, and KEGG pathways were detected using the R/Bioconductor package GAGE9 to test for changes in expression of the reported log 2 fold-changes reported by Limma in each term versus the background log 2 fold-changes of all genes found outside the respective term. The R/Bioconductor package heatmap3 was used to display heatmaps across groups of samples for each GO or MSigDb term with a Benjamini-Hochberg false-discovery rate adjusted p-value less than or equal to 0.05. Perturbed KEGG pathways where the observed log 2 fold-changes of genes within the term were significantly perturbed in a single-direction versus background or in any direction compared to other genes within a given term with p-values less than or equal to 0.05 were rendered as annotated KEGG graphs with the R/Bioconductor package Pathview.

To find the most critical genes, the Limma voomWithQualityWeights transformed log 2 counts-per-million expression data was then analyzed via weighted gene correlation network analysis with the R/Bioconductor package WGCNA. Briefly, all genes were correlated across each other by Pearson correlations and clustered by expression similarity into unsigned modules using a power threshold empirically determined from the data. An eigengene was then created for each de novo cluster and its expression profile was then correlated across all coefficients of the model matrix. Because these clusters of genes were created by expression profile rather than known functional similarity, the clustered modules were given the names of random colors where grey is the only module that has any pre-existing definition of containing genes that do not cluster well with others. These de-novo clustered genes were then tested for functional enrichment of known GO terms with hypergeometric tests available in the R/Bioconductor package clusterProfiler. Significant terms with Benjamini-Hochberg adjusted p-values less than 0.05 were then collapsed by similarity into clusterProfiler category network plots to display the most significant terms for each module of hub genes to interpolate the function of each significant module. The information for all clustered genes for each module were then combined with their respective statistical significance results from Limma to determine whether those features were also found to be significantly differentially expressed.

Statistical analysis: Graphpad Prism 8.0 was used to perform all statistical analyses. Data are presented as mean±SEM unless otherwise stated. Data were checked for normality using the Shapiro-Wilk method, D'Agostino and Pearson normality test as well as KS normality test. Statistical significance between groups with normally distributed data was calculated by unpaired T-test or two-way ANOVA followed by Tukey's post hoc test for group-wise comparisons unless stated otherwise. P less than 0.05 was considered significant: *p<0.05, **p<0.01, and ***p<0.001.

### Example 3: Effects of Lemborexant and Doxepin in a Model of Aβ Burden

### A. Sleep-Wake Behavior

Changes in sleep were assessed in APPswe/PS1deltaE9 (also called “PSAPP”) mice after administration of either doxepin or lemborexant. Briefly, 4-5 mo PSAPP mice (mixed sexes) were single housed and the cages were placed on the SignalSolutions piezoelectric sleep monitoring base (Adapt-A-Base). Sleep-wake behavior was monitored for 7 days while mice were treated with vehicle, doxepin 35 mg/kg, or lemborexant (10 or 30 mg/kg) via oral gavage every day at ZT 0 (light on).

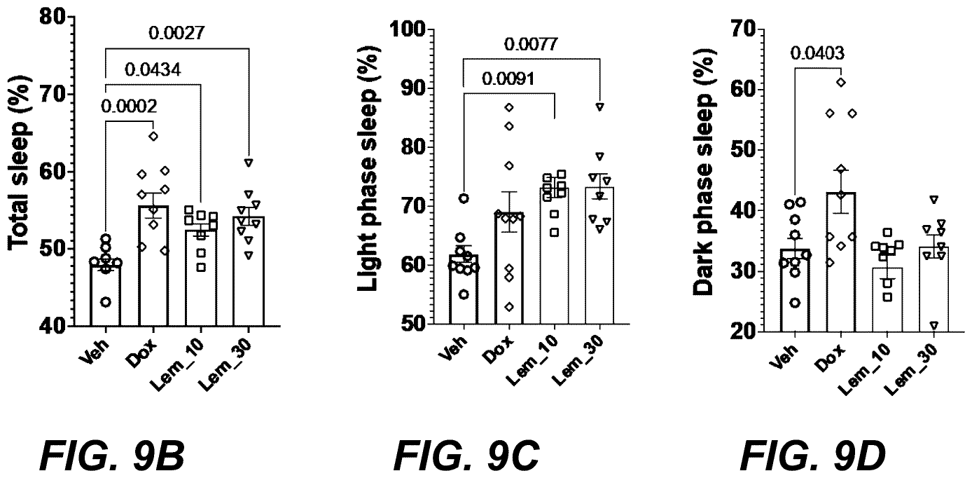

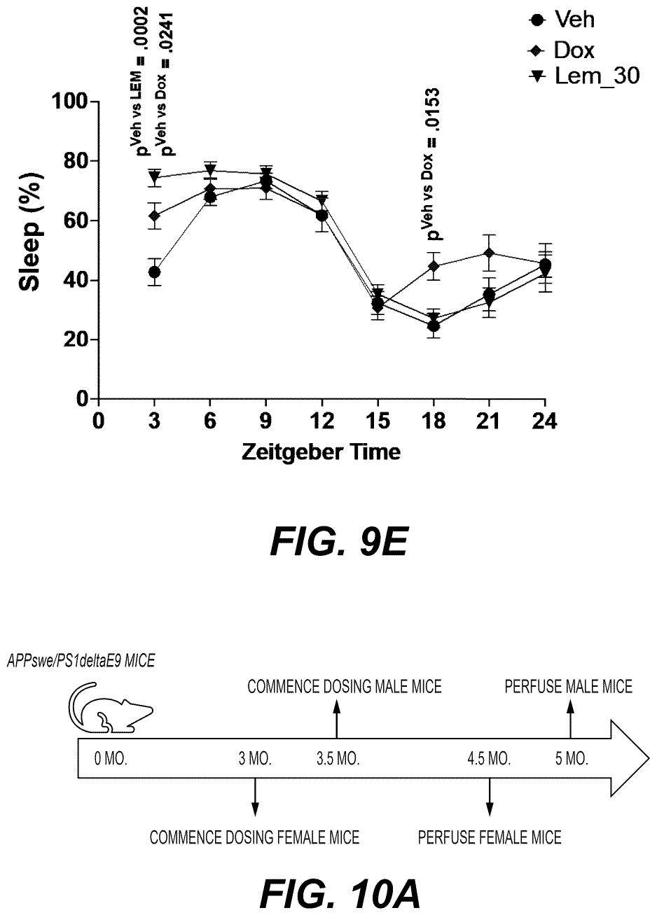

FIG. 9A shows a schematic of the experimental design and graphs of the effects of doxepin and lemborexant on total sleep (FIG. 9B), light phase sleep (FIG. 9C), and dark phase sleep (FIG. 9D). P values from 1-way ANOVA are shown. Each dot represents one mouse. FIG. 9E shows the sleep percentage across time-of-day timepoints after drug injection. P values are from 2-way repeated measures ANOVA.

The data indicate that lemborexant and doxepin each increase total sleep time in PSAPP mice, and the effects are comparable after treatment with each drug. Notably, DOX-induced sleep is spread throughout the day, whereas LEM induced sleep is limited to light phase (natural rest phase for mice).

### B. Amyloid Plaque Deposition

Either lemborexant or doxepin were chronically administered to PSAPP mice in order to determine the long-term effects of each drug effects on amyloid plaque deposition. PSAPP mice were treated with drug by oral gavage 6 days/week at ZT0 (lights on) for 1.5 mo.

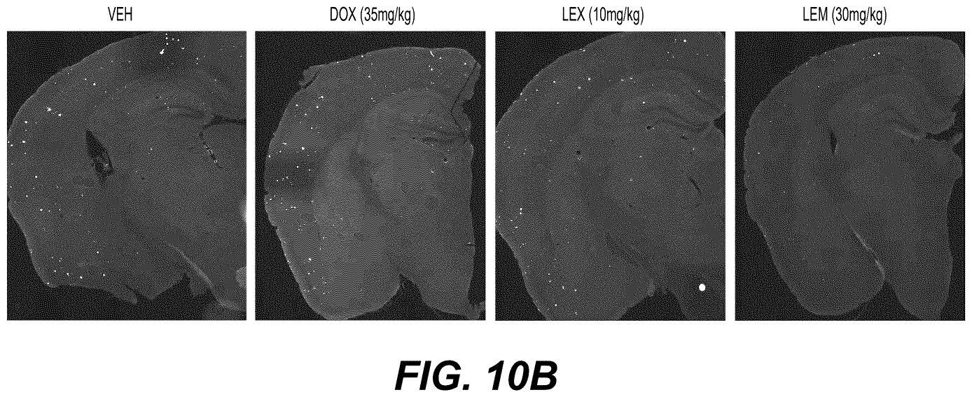

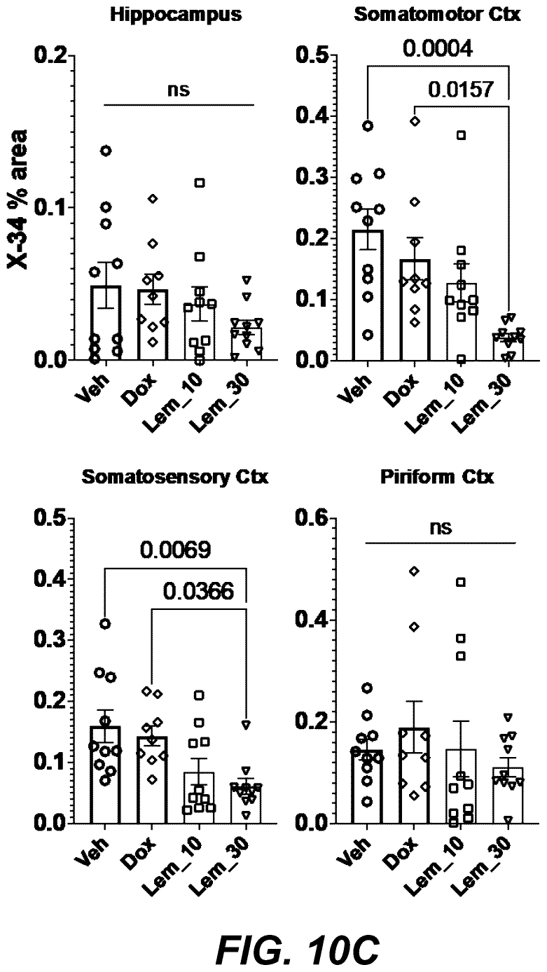

FIG. 10A shows a schematic for the timing of treatment of PSAPP mice. Females develop plaques faster/younger, so males and females were staggered to allow combination of data. FIG. 10B shows representative images of brain sections stained with X34, which labels fibrillar amyloid plaques. FIG. 10C shows the quantification of plaque burden (% area X34 staining) in different brain regions (hippocampus, somatomotor cortex, somatosensory cortex, and piriform cortex). Error bars indicate mean±SEM, each dot is a mouse. P values from 1-way ANOVA are shown. The data indicate that chronic administration of lemborexant reduces fibrillar amyloid plaque burden in PSAPP mice, while doxepin did not. A dose response of the effects was observed for lemborexant.

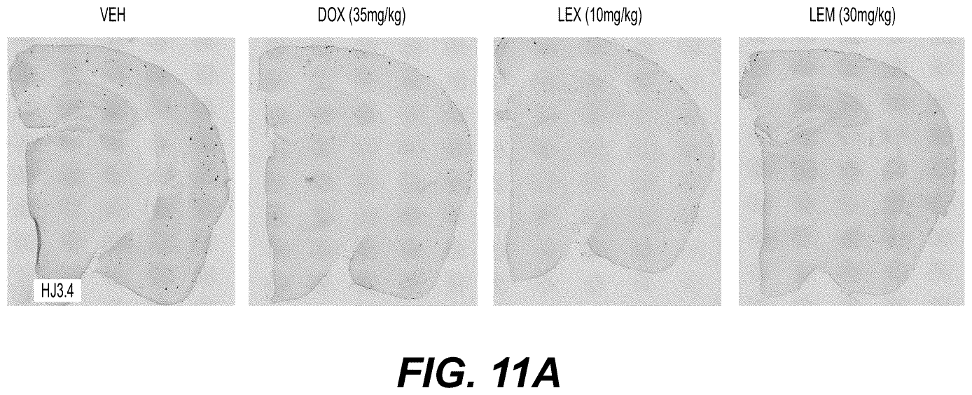

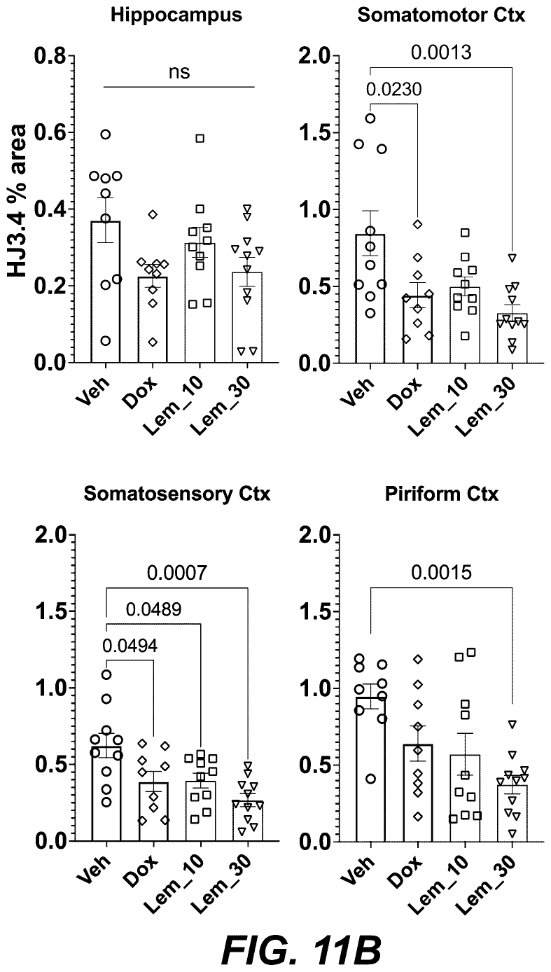

FIG. 11 shows that chronic lemborexant reduces total amyloid plaque burden in PSAPP mice more effectively than doxepin. FIG. 11A shows brain sections stained for total amyloid plaques burden using the anti-Ap antibody HJ3.4. FIG. 11B shows the quantification of plaque burden (measured as the % area showing HJ34 staining) in different brain regions (hippocampus, somatomotor cortex, somatosensory cortex, and piriform cortex). Error bars indicate mean±SEM, each dot is a mouse. P values from 1-way ANOVA are shown.

Taken together, the data show that lemborexant reduces total amyloid plaque burden (including both diffuse and fibrillar plaques) in PSAPP mice. Doxepin also significantly lowers total amyloid burden, but not fibrillar plaque burden, and not to the same degree as lemborexant (at a 30 mg/kg dose), as only lemborexant (30 mg/kg dose) shows a significant effect in the piriform cortex.

The differential response of lemborexant as compared to doxepin suggests that the effects on plaque development are separable from the similar sleep effects induced by these two drugs.

### C. Amyloid Processing

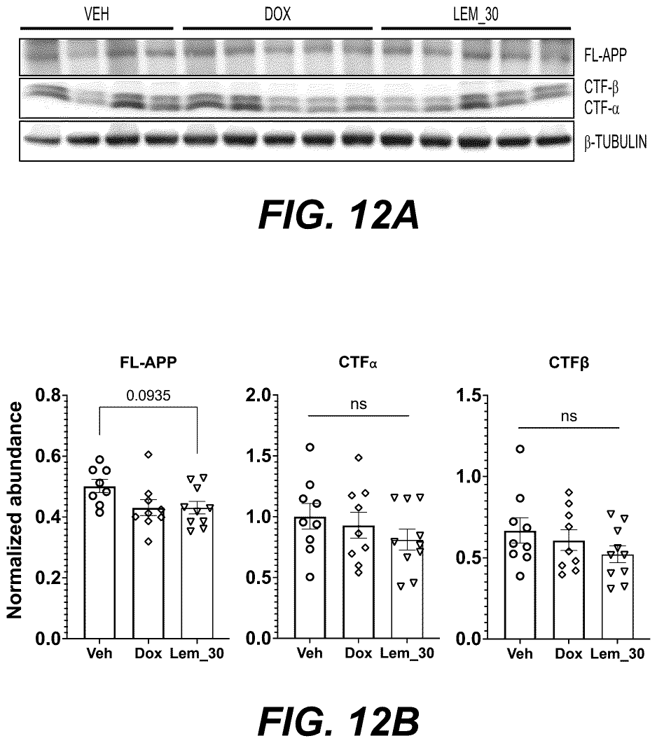

To determine whether lemborexant and doxepin affect amyloid processing, cortical lysates were obtained from PSAPP mice treated with either drug and levels of full-length APP and APP C-terminal fragments (CTF) were measured by Western blot. FIG. 12A shows representative Western blots, with beta-tubulin as a loading control. FIG. 12B shows the quantification of band intensity. Error bars indicate mean±SEM, each dot is a mouse. P values from 1-way ANOVA are shown.

The results show that neither lemborexant nor doxepin alter APP processing/cleavage in PSAPP mice.

### D. Size of Amyloid Plaques and Number of Microglia Surrounding Plaques

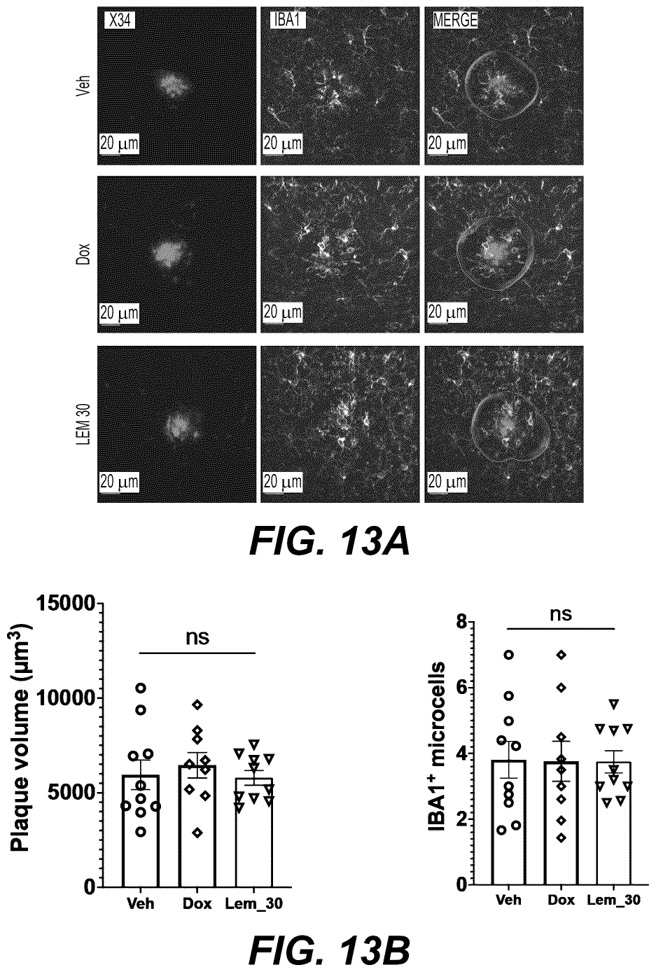

The number of microglia surrounding amyloid plaques were determined in PSAPP mice after administration of either lemborexant or doxepin. Brain sections from PSAPP mice were stained for plaques (using X34) and microglia (using Iba1). Plaques were selected that were approximately the same size across all mice. The volume of microglia around each plaque was calculated from Z-stacks of confocal images using Imaris software. FIG. 13A shows representative images from brain section obtained from PSAPP mice after administration of either lemborexant or doxepin. FIG. 13B shows the quantification of plaque volume, indicating that similar sized plaques were quantified across conditions, and peri-plaque Iba1 volume. Error bars indicate mean±SEM, each dot is a mouse. P values from 1-way ANOVA are shown.

The results indicate that the number of microglia surrounding plaques are unchanged after administration of either lemborexant or doxepin.

### E. Phagocytic Subtypes of Microglia Surrounding Fibrillar Amyloid Plaques

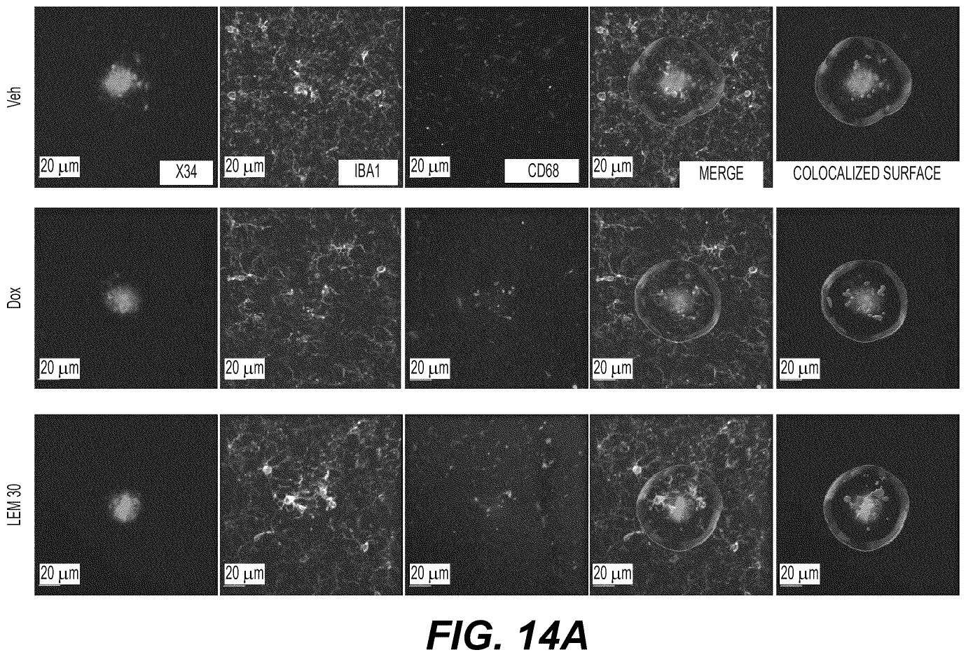

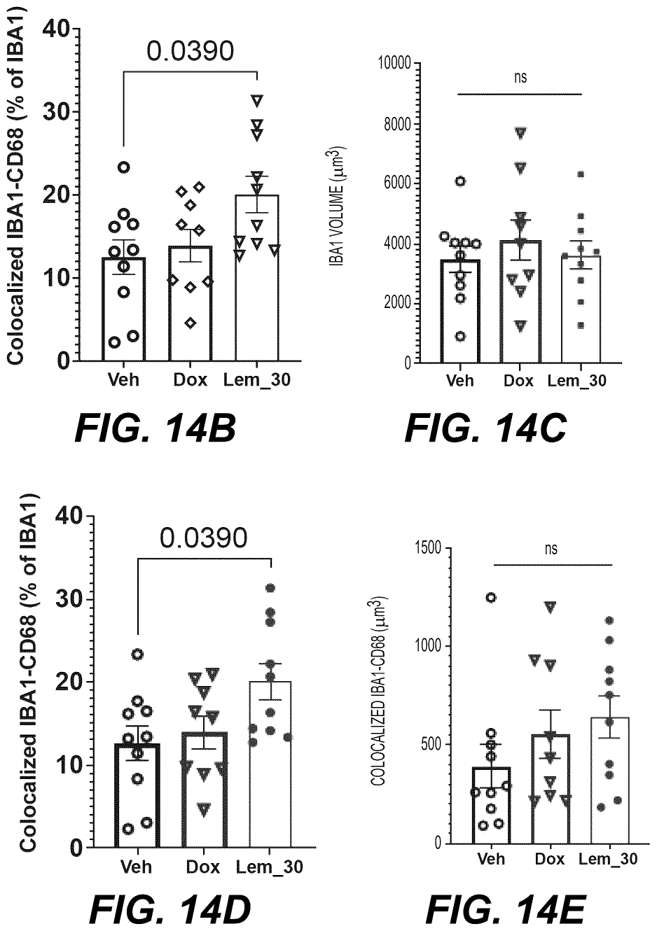

To determine the subtypes of microglia surrounding fibrillar amyloid plaques, CD68 expression was determined in microglia of PSAPP mice after administration of either lemborexant or doxepin. Brain sections from PSAPP mice were stained with a marker for fibrillar plaques (X34), microglia (Iba1), and phagosomes (CD68). FIG. 14A shows representative images of brain sections. FIG. 14B shows the quantification of Iba1-colocalized CD68 around each plaque, obtained using Imaris software. Error bars indicate mean±SEM, each dot is the average of 8-10 plaques from a single mouse. P values from 1-way ANOVA are shown.

The results indicate that the administration of lemborexant, but not doxepin, increases phagocytic subtypes of microglia surrounding fibrillar amyloid plaques. Total microglia counts are unchanged with regards to area density and the total number of microglia co-localizing to plaques is unchanged. FIG. 14C shows Iba1+ volume, FIG. 14D shows the co-localized Iba1+ and CD68+ (as a percentage of Iba1+ staining), and FIG. 14E shows the co-localized Iba1+ and CD68+. Increased CD68 in microglia around plaques indicates increased phagocytic activation.

### F. Gene Expression

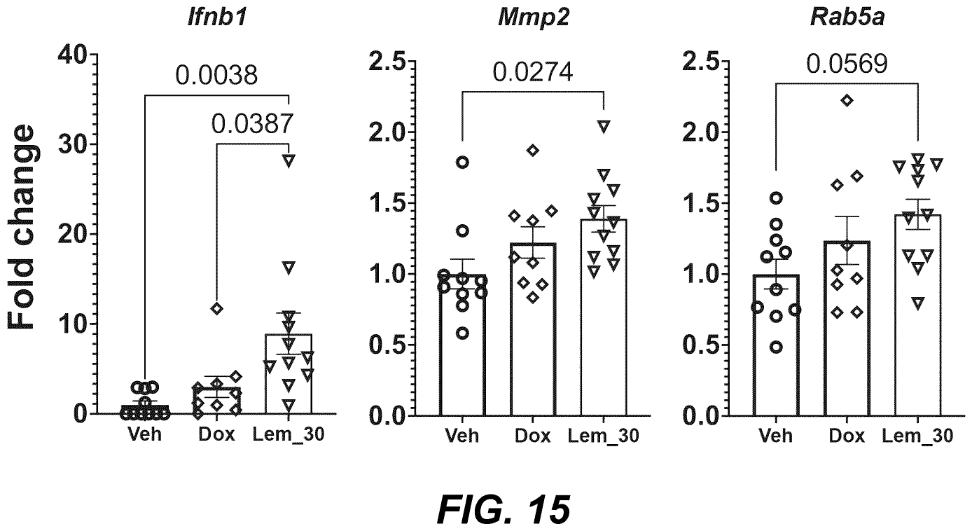

To identify changes in gene expression following administration of lemborexant or doxepin to PSAPP mice, qPCR array was performed on cortical tissue from the mice. FIG. 15 is a quantitative plot of three transcripts which showed significant differences in expression. Ifnb1 encodes the inflammatory mediator IFN-beta, which is implicated in microglia regulation in AD. Rab5a encodes a lysosomal protein, and Mmp-2 encodes a metalloprotease which has been shown to degrade Aβ. Data is displayed as fold change (relative to the mean for VEH). Error bars indicate mean±SEM, each dot is a single mouse. P values from 1-way ANOVA are shown.

The results show that genes associated with Aβ degradation are upregulated in PSAPP mice after administration of lemborexant. The gene expression changes were not significant in the doxepin-treated group.

### G. Number of Microglia Actively Phagocytosing Amyloid Plaques

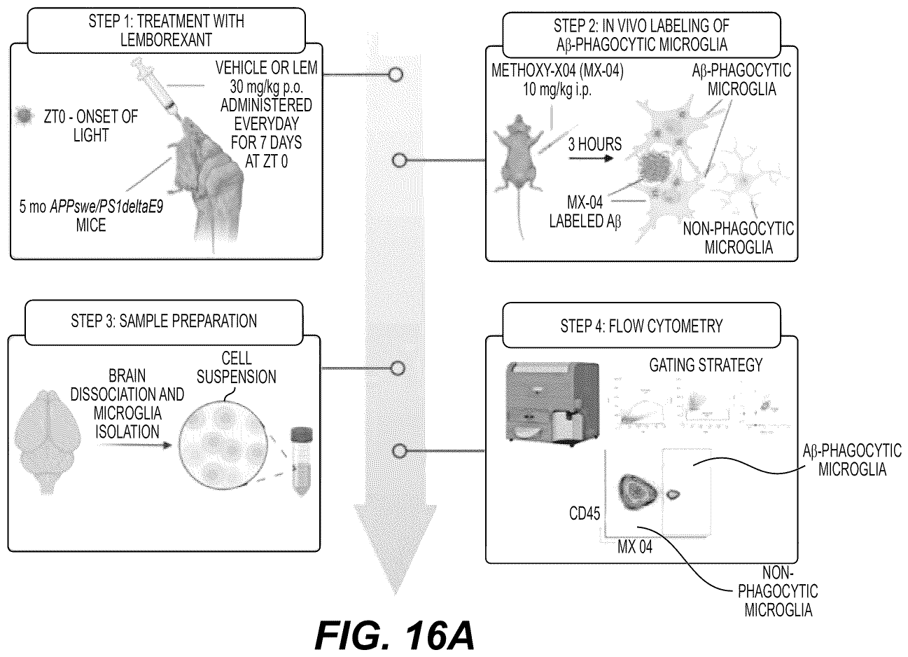

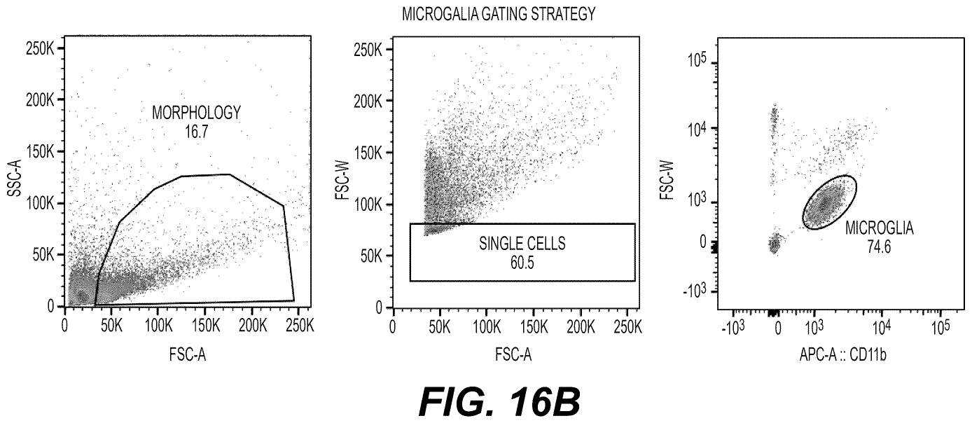

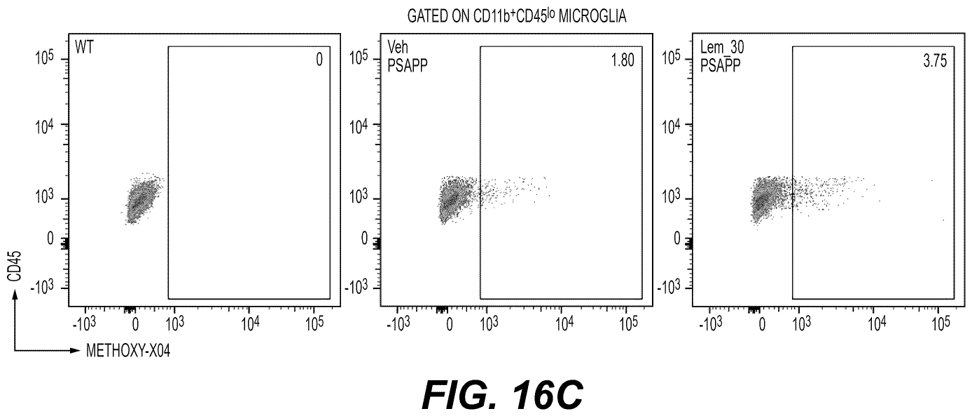

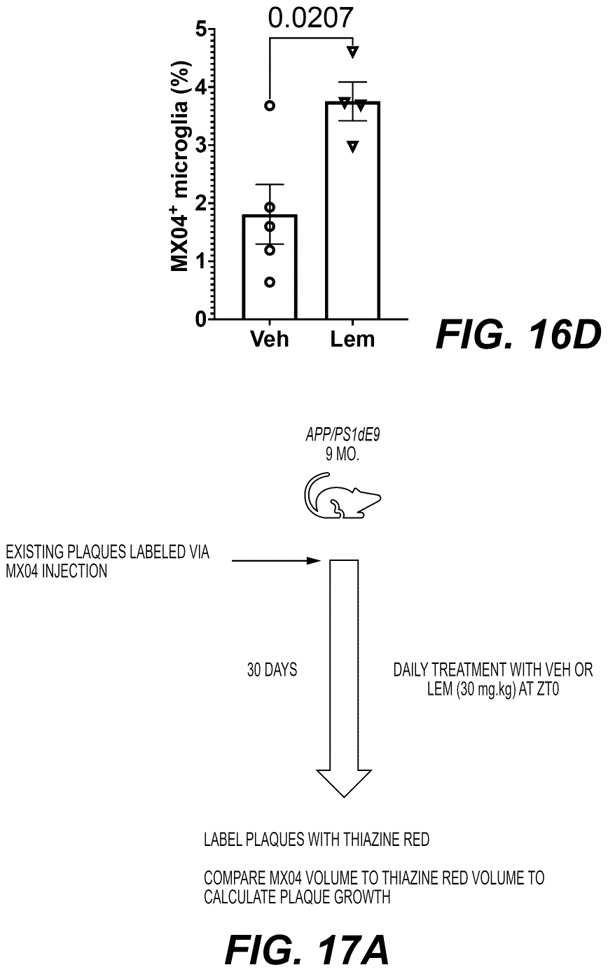

The effect of lemborexant on microglial amyloid plaque phagocytosis was assessed in PSAPP mice. FIG. 16A shows a schematic of the experimental design (Lau et al., STAR Protoc. 2021). Briefly, 5 month old PSAPP mice were treated daily with vehicle (Veh) or Lemborexant (LEM) 30 mg/kg by oral gavage for 7 days. After treatment the 7th day, amyloid plaques were labeled in vivo via intraperitoneal (i.p.) injection of methoxy-X04 (MX04). Three hours later, mice were sacrificed and microglial were isolated from the brain and subjected to flow cytometry. FIG. 16B show a flow cytometry gating strategy to isolate likely microglia. Following side and forward scatter gating to identify viable single cells, the CD45-low, CD11b+ population was isolated as likely microglia. FIG. 16C shows the analysis of this cell population for methoxy-X04 positivity. FIG. 16D shows the quantification of the percentage of MX04+ microglia, indicating microglia which have phagocytosed labelled amyloid. P=0.0207 by 2-tailed T-test.

The results show that LEM treatment acutely increases microglial amyloid plaque phagocytosis in vivo. MX04+ cells were not observed in wild-type mice that lack amyloid plaques. In contrast, 1.8% of microglia had ingested MX04 in the vehicle-treated PSAPP mice, and 3.75% of microglia had ingested MX04 in the LEM-treated group.

### H. Amyloid Plaque Deposition in Aged Subjects

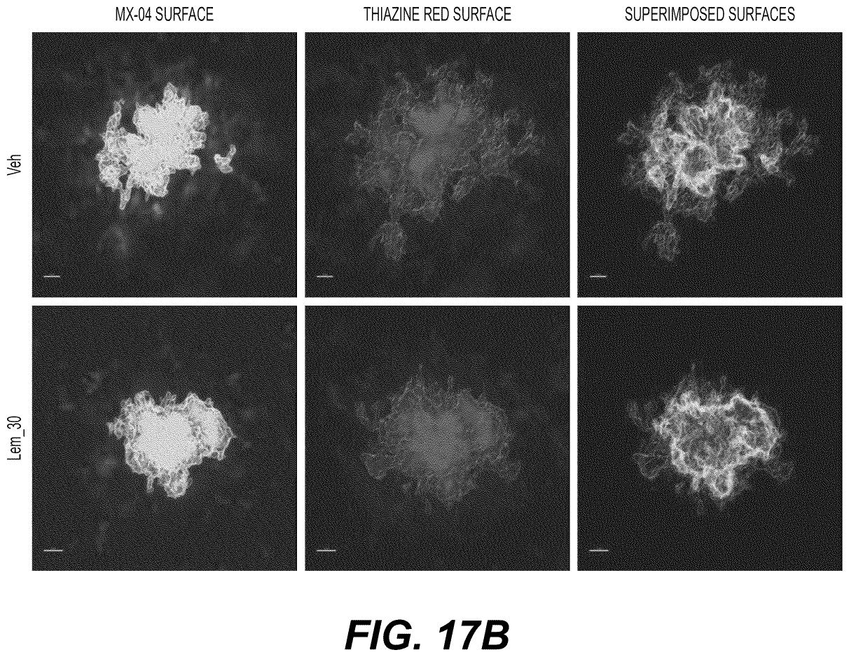

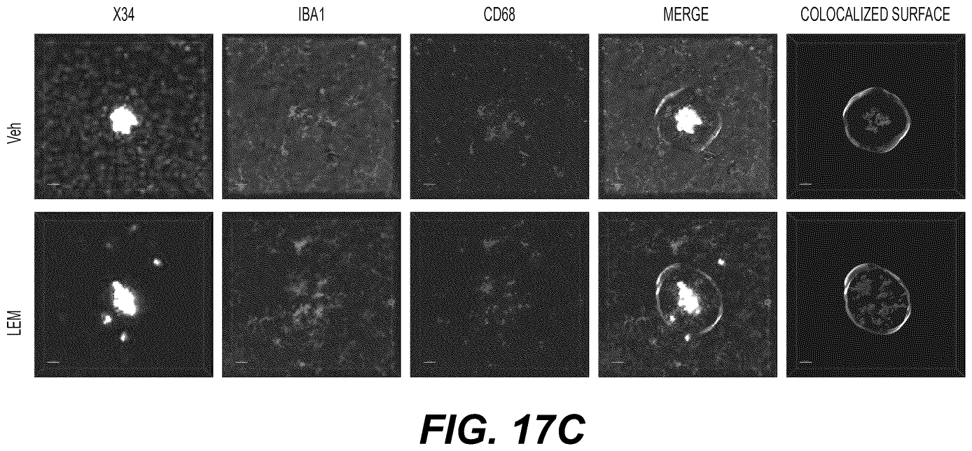

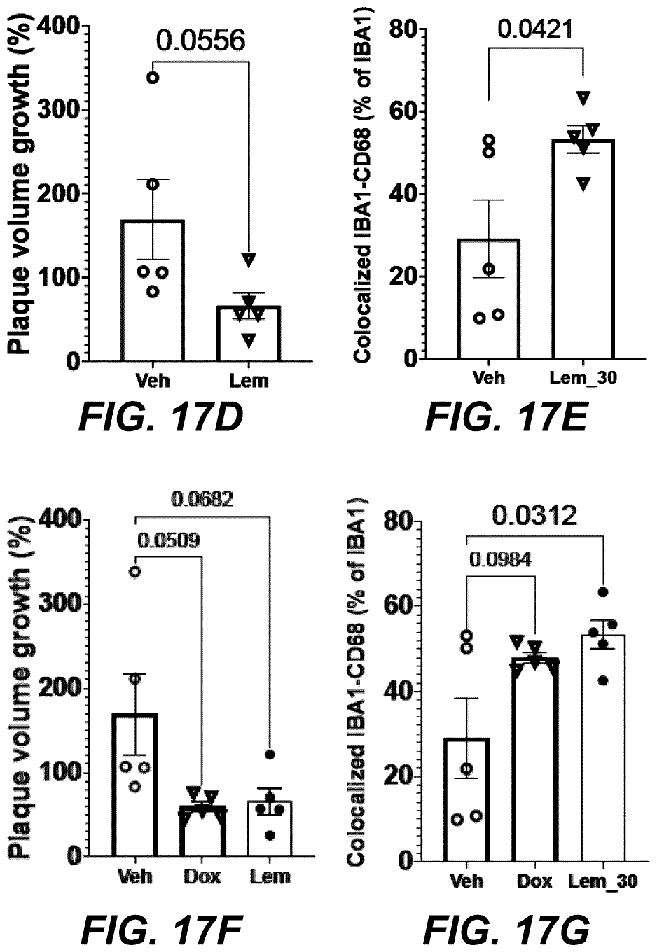

The effects of administering lemborexant were assessed in aged PSAPP mice. FIG. 17A shows a schematic of the experimental design. Briefly, 9-month-old PSAPP mice with amyloid plaques were treated daily with vehicle (Veh), or Lemborexant (LEM, 30 mg/kg)) for 30 days. One day 1 of treatment, existing plaques were labeled in vivo via intraperitoneal (i.p.) injection of methoxy-X04 (MX04). After 30 days of treatment, mice were sacrificed and all plaques were labeled with Thiazine red, which is similar to X34 and bind fibrillar amyloid. Plaque growth during the 30 day treatment period was calculated by comparing MX04 volume to thiazine red volume on a plaque-by-plaque basis. FIG. 17B shows representative images of MX04, thiazine Red, and overlay images. FIG. 17D shows a graph where at least 10 plaques were analyzed per mouse and the average per mouse is shown with each circle on the graph. The LEM-treated group showed a trend toward reducing the amount of plaque growth during the 30 days of treatment. P values are from Mann-Whitney U test, due to non-Gaussian distribution of data. FIG. 17C shows representative images of amyloid plaques labeled with X34, microglia labeled with IBA1, and microglial phagosome labeled with CD68. The IBA1-CD68 colocalized volume was calculated within a 20 μm sphere from each amyloid plaque to determine peri-plaque microglial CD68 expression. FIG. 17E shows quantification of colocalized IBA1-CD68, displayed as a percent of total IBA1 (total microglial) area. Each circle represents the average for a single mouse, with 10 plaques quantified per mouse. In FIG. 17D and FIG. 17E, the comparison between VEH and LEM treated groups was analyzed by two tailed t-test.

The effects of administering doxepin were also assessed in aged PSAPP mice. Neither lemborexant nor doxepin administration led to a significant change in total plaque number in aged mice. A similar lack of significant change was observed in plaque volume, IBA1+ cells, IBA1 volume, and co-localization of IBA1-CD68.

Finally, dystrophic neurite volume surrounding the amyloid plaques was quantified by measuring BACE1 elevation in pre-synaptic termini. Neither lemborexant nor doxepin administration led to a significant change in the dystrophic neurite volume.

The effects of doxepin administration on plaque growth and phagocytic microglia surrounding amyloid plaques are shown together with data from lemborexant administration in FIG. 17F and FIG. 17G. In FIG. 17F and FIG. 17G the comparison of VEH, DOX, and lemborexant-treated groups was analyzed by one way ANOVA.

Taken together, the results show that lemborexant and doxepin each show a trend towards slowing amyloid plaque growth in aged mice with pre-existing plaques. Each drug increases phagocytic microglia surrounding amyloid plaques in aged mice. Lemborexant, but not doxepin, also shows this effect on phagocytic microglia in younger mice. Both lemborexant and doxepin showed less impact on removing established plaques in older mice than on preventing plaque accumulation in younger mice.

### I. Methods

### J. Animals

Male and female APPswe/PS1deltaE9 (PSAPP) mice were used for all experiments.

APP/PS1 are double transgenic mice expressing a chimeric mouse/human amyloid precursor protein (Mo/HuAPP695swe) and a mutant human presenilin 1 (PS1-dE9), both directed to CNS neurons.

### K. Methods for Measuring Sleep-Wake Behavior

5-month-old APPswe/PS1deltaE9 (PSAPP) mice were dosed per oral with VEH, LEM (10 mg/kg/day or 30 mg/kg/day), or DOX (35 mg/kg/day) at ZT0 each day for 6 days. Sleep and wake states were determined using a noninvasive piezoelectric system, Adapt-A-Base (Signal Solutions). Mice were individually housed in cages placed over the piezo sensor base in sound- and light-proof cabinets (Circadian cabinets, ClockLab) and recorded without disturbance over 7 days. At the beginning of recordings, each cage was furnished with 160 g corncob bedding, 220 g food pellets, and 380 mL water, so that each cage weighed the same except for a small variability between mouse body weight. The sleep-wake states were analyzed by SleepStats software (Signal Solutions). 30-second epochs were used for scoring sleep bout lengths following the manufacturer's default setting in the current version of SleepStats software.

### L. Methods for Assessing Effects of Long-Term Administration of Lemborexant or Doxepin on Amyloid Plaque Deposition

Young PSAPP mice (3-month old females and 3.5-month old males) were dosed p.o. with VEH, LEM (10 mg/kg/day or 30 mg/kg/day), or DOX (35 mg/kg/day) at ZT0 each day for 6 weeks. Subsequently, female and male mice were perfused at 4.5-mo and 5-mo, respectively. Brains were extracted and processed for IHC/IF, transcriptomics, or proteomics analyses.

### M. Methods to Assess the Effects of Lemborexant and Doxepin on Microglial Aβ-Phagocytic Activity

5-mo PSAPP mice were dosed with VEH, or LEM (30 mg/kg/day) at ZT0 each day for 7 days. On the 7th day, mice were injected i.p. with methoxyX-04 (MX-04, 10 mg/kg) to label plaques in vivo. Mice were perfused 3 hr. post-MX04 injections, and brains were processed for flow cytometry assay for estimation of MX-04 positive microglial cells.

### N. Method for Assessment of Effects of LEM on Amyloid Plaque Deposition in Aged PSAPP Mice

9-mo PSAPP mice were injected intraperitoneally (i.p.) with methoxyX-04 (MX-04, 10 mg/kg) to label plaques in vivo and dosed per oral with VEH or LEM (30 mg/kg/day) at ZT0 each day for 4 weeks. Mice were perfused at 10-mo and brains were extracted and processed for IHC/IF analyses. Fixed brain sections were stained with thiazine red and compared to MX-04 staining to estimate plaque growth.

### O. Drugs

Mice were dosed per oral with either vehicle (VEH), Doxepin (DOX, Cayman Chemical #15888, solubilized in PBS), or Lemborexant (LEM, suspended in 0.5% methylcellulose) at ZT0 (lights on). VEH mice were given similar volumes of vehicle each day. The tip of the 22-gauge oral gavage needle was dipped in 100% sucrose solution immediately prior to gavaging. MethoxyX-04 (Tocris #4920, 10 mg/kg) was injected i.p. for the timestamp experiment and for analyses of microglial phagocytic activity.

### P. IHC/IF:

All mice were perfused between ZT5 and ZT7 (1100 and 1300 hours). Mice were deeply anesthetized with i.p. pentobarbital (150 mg/kg), then perfused transcardially with ice-cold Dulbecco's modified PBS (DPBS) containing 3 g/l heparin. The brains were carefully extracted and the left hemisphere was post-fixed in 4% paraformaldehyde for 48 hours (4 °C), then cryoprotected with 30% sucrose in PBS (4 °C) for 24 hours. Brains were then sectioned on a freezing sliding microtome (SM1020R; Leica) in 40 μm serial coronal sections and stored in cryoprotectant solution (30% ethylene glycol, 15% sucrose, 15% phosphate buffer in ddH20). For biochemical analysis, the right hemispheres were dissected to isolate the cortex and the hippocampus, flash frozen, and kept at −80 °C until analyzed.

Fibrillar Aβ was stained by X-34 dye (SML-1954, 1:5,000; MilliporeSigma) or Thiazine Red. For X-34 staining, free-floating sections were washed 3 times in PBS for 5 minutes each and then permeabilized in 0.25% Triton X-100 PBS (PBS-X) for 30 minutes. Tissue sections were then incubated in X34-CA M NaOH for 20 minutes, washed in X34 buffer (40% EtOH in PBS), and then washed twice in PBS. To stain for total Aβ (HJ3.4 biotinylated, anti-Aβ1-13, mouse monoclonal, 1:1,000, 2.81 μg/mL; generated in-house), sections were washed in TBS×3, then incubated in 0.3% hydrogen peroxide for 10 minutes. Sections were washed again in TBS×3, then blocked in 3% milk diluted in TBS+0.25% Triton X-100 for 30 minutes. Sections were incubated overnight in biotinylated HJ3.4 in TBS+0.25% Triton X-100+1% milk at 4 °C. The next day, sections were washed and then developed using ABC Elite (Vector PK-6100) for 60 minutes. Sections were then incubated in 3,3-diaminobenzidine (DAB, Sigma-Aldrich) as chromogen and 0.05% hydrogen peroxide as substrate, and dehydrated before coverslipping using Cytoseal 60 (8310: Thermo Fisher Scientific).

For immunofluorescence (IF) staining with IBA1 (goat, Abcam ab5076, 1:500) or CD68 (rat, BioRad MCA1957, 1:500), sections were washed in TBS×3, blocked for 60 minutes in TBSX (TBS+ 0.4% Triton X-100) containing 3% donkey serum, and incubated overnight at 4 °C in primary antibodies diluted in TBSX containing 1% donkey serum. Sections were then incubated for 1 hour at room temperature in PBSX (or TBSX) with 1:1000 donkey fluorescent secondary antibody. Sections were mounted, sealed in Fluoromount-G (0100-01; SouthernBiotech), and stored in the dark at 4 °C until imaging.

### Q. Imaging

Epifluorescence imaging: All fluorescent imaging was done on a Keyence BZ-X810 microscope. In general, laser intensity and exposure times were selected for each cohort of samples after a survey of the tissue, in order to select appropriate parameters that could then be held constant for all slides in that imaging session. These values varied by antibody, but all sections in a given cohort were imaged under identical conditions at the same magnification. Images were processed using BZ-X800 Analyzer program (Keyence Corp.) and quantified with the use of Fiji version 2.1.0 (NIH). All areas were quantified in 2-3 sections per mouse.

Confocal imaging and analyses: Plaques located in the cortical grey matter and fully contained within the slice thickness were selected for imaging. 16-bit image stacks were acquired in a sequential mode at 0.26 μm z-steps using a Zeiss LSM-980 Airyscan 2 confocal microscope and the ZEN software (v 3.7, blue edition). Uniform pinhole, laser power, and PMT detector gain settings were used in all experiments. For all analyses, at least 2 images were taken per brain region and slide using a 40× oil immersion objective lens at a minimum 1,024×1,024 resolution. Quantification of confocal images for IBA1 and CD68 volume around X34+ plaques was performed on a semiautomated platform using MATLAB and Imaris 10.0.1 software (Bitplane). To create surfaces of each stain based on a threshold applied to all images, X34+ surfaces were dilated by 20 μm and colocalized with various immunostained surfaces. IBA1+ and CD68+ surface areas were then colocalized within the 20 μm extended shell around the plaques. For quantification of the number of plaque-associated IBA1+ microglia, a threshold was applied across all images to assign spots to each cell body or punctum. X34 surfaces were dilated to 20 μm, and spots were counted within the X34+ dilated surface. Any spots fully within or partially touching the extended surface were included in the analysis.

### R. Western Blotting

Tissue samples were homogenized by sonication on ice in radioimmunoprecipitation (RIPA) buffer (Pierce, Thermo Scientific) containing complete protease inhibitors and PhosSTOP phosphatase inhibitors (Roche). PAGE and Western blotting were performed using Invitrogen Novex gels and reagents. Bands were visualized using Lumigen TMA-6 chemiluminescence reagents on an iBright CL1500 Imaging System (Thermo). Band intensity was quantified using Fiji software (NIH) and was normalized to the β-tubulin loading control.

### S. Statistics

Statistical tests were performed and graphs were plotted using GraphPad Prizm software, version 9.0.1. Power analysis was used to determine the variability of outcome measures and to estimate the number of observations necessary to test the null hypothesis. All graphical representations display the mean with bar graphs, individual data points within the bar, and error bars depicting SEM. For experiments 1 and 2, one-way ANOVA was performed. If the main effect was significant, then following post-hoc multiple comparison tests were performed: Tukey (for equal group sizes) or Tukey-Kramer (for unequal group sizes). For experiments 3 and 4, an F test was first performed for datasets with a single dependent variable and 2 groups, to determine if variances were significantly different. If not, a 2-tailed unpaired t-test was performed. If variances were different, a non-parametric Mann-Whitney U test was performed. Outliers were identified using the Grubbs test and were excluded. All P values are noted in the figures.

### Example 4: Effects on Rhythmic Activity Patterns

The effects of lemborexant administration on rhythmic activity patterns was evaluated in arrhythmic Bmal1 knockout (KO) mice in order to assess the restorative effect of lemborexant on mice lacking a functioning clock. Bmal1 KO mice lack a functioning clock and consequently lose their 24-hour rhythms in constant darkness (DD condition). The arhythmic behavior of Bmal1 KO mice is masked in the normal 12-hour light:12-hour dark cycle (L:D condition).

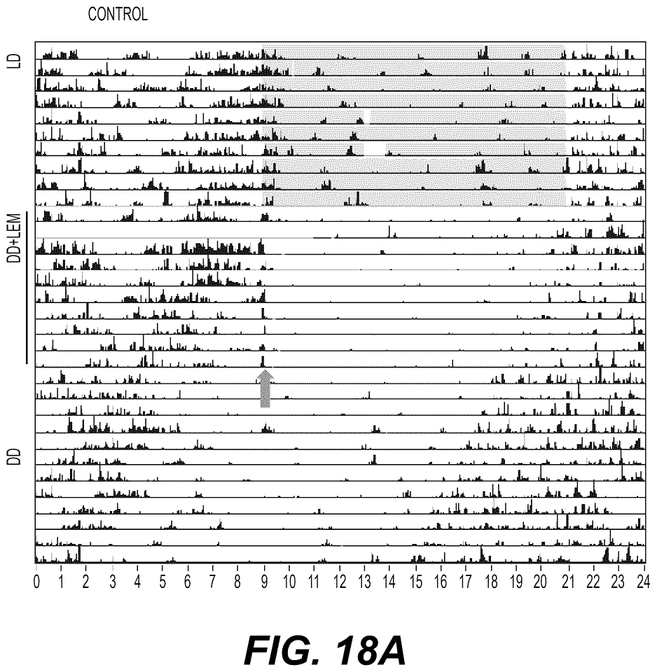

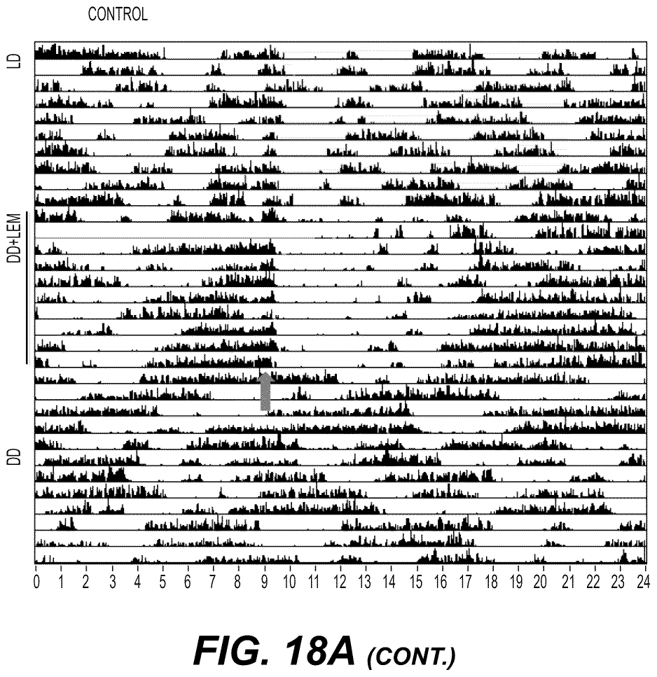

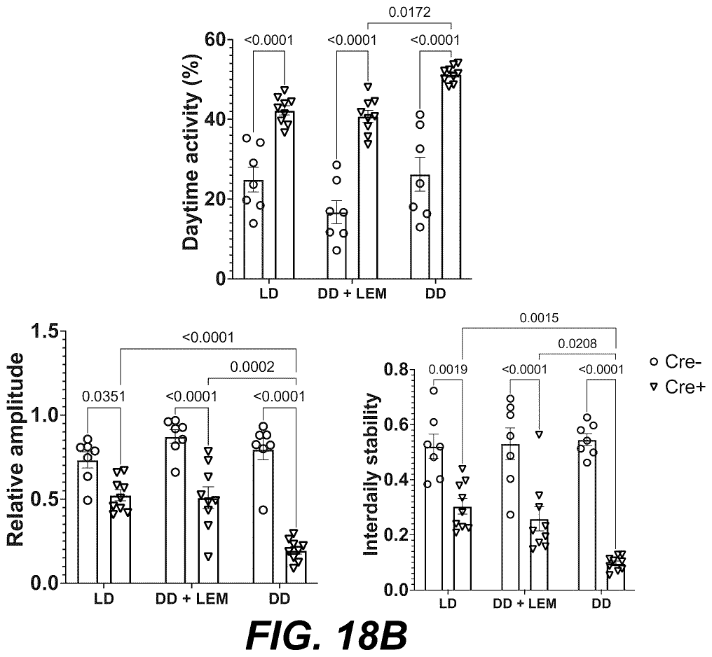

Control (Cre-) or Bmal1 KO (CAG-CreERT2; Bmal1(f/f)) mice were treated with tamoxifen to delete Bmal1. One month later infra-red actigraphy was recorded under 12 h:12 h L:D condition (yellow area) or constant darkness (DD condition). Mice were administered lemborexant (LEM) at a dose of 30 mg/kg by oral gavage at 6 am (previous ZT 0) each day for 9 days (indicated by red bar and red arrow). Actigraphy was collected for 2 more weeks after dosing ceased. FIG. 18A shows representative actograms for control and Bmal1 knockout mice. FIG. 18B is a quantification of actigraphic endpoints during different portions of the experiment (in FIG. 18A, LD indicates during yellow area, DD+LEM is area with the red bar, and DD is the rest of the recording). Data analyzed by 2-way ANOVA with Tukey post-hoc test.

The results indicate that Bmal1 knockout mice showed greater daytime activity than wild-type mice under all conditions (LD, DD+LEM, and DD). Bmal1 KO mice are able to maintain their locomotor activity in DD conditions similar to LD when they are given lemborexant daily at CTO (CTO is similar to ZT0 in DD conditions). Relative amplitudes, which indicate the ratio of average activity in the most active 10 hours as compared to average activity in the least active 5 hours, were lower in Bmal1 knockout mice under all conditions. In general, higher relative amplitudes correlate with stable rhythms. Interdaily stability (IS), which measures the synchronization between daily 24-hour rhythms, was lower in Bmal1 knockout mice under all conditions. High IS indicates good synchronization of rhythms.

### EQUIVALENTS AND SCOPE

Those skilled in the art will recognize, or be able to ascertain using no more than routine experimentation, many equivalents to the specific embodiments in accordance with the Detailed Description provided herein. The scope of the present disclosure is not intended to be limited to the above Detailed Description, but rather is as set forth in the appended claims.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any, composition, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method for treating Alzheimer's disease (AD) in a subject who has AD or is at risk of developing AD, comprising administering to the subject a therapeutically effective amount of lemborexant, a pharmaceutically acceptable salt thereof, or a solvate thereof, thereby treating AD.

2. The method of claim 1, wherein treating AD comprises reducing and/or slowing cognitive decline.

3. The method of claim 1, wherein treating AD comprises affecting a change (e.g., slowing, delaying, or reducing) in at least one marker of AD pathology.

4. The method of claim 3, wherein the marker is a level of phosphorylation of tau, neurodegeneration, a change in microglial response, and/or presence of Aβ plaques.

5. The method of claim 4, wherein the marker is present in a brain region in the subject.

6. The method of claim 5, wherein the brain region is the hippocampus, somatomotor cortex, somatosensory cortex, piriform cortex, and/or entorhinal cortex.

7. The method of claim 4, wherein the marker is detected in a body fluid of the subject.

8. The method of claim 7, wherein the body fluid is blood or cerebrospinal fluid (CSF).

9. The method of claim 1, wherein the subject does not show signs of dementia and/or cognitive impairment.

10. The method of claim 1, wherein the subject has mild cognitive impairment or mild dementia.

11. The method of claim 1, wherein the subject is amyloid positive.

12. The method of claim 11, wherein the subject is at risk for further Aβ accumulation.

13. The method of claim 12, wherein the subject is an ApoE4 carrier.

14. The method of claim 12, wherein the subject has intermediate levels of amyloid PET (e.g., 20-40 centiloids).

15. The method of claim 12, wherein the subject has elevated levels of amyloid PET (e.g., >40 centiloids).

16. The method of claim 1, wherein the subject has been diagnosed with AD, based on brain imaging, cognitive function, and/or biomarker criteria.

17. The method of claim 16, wherein the subject has early AD.

18. The method of claim 16, wherein the subject has pre-AD.

19. A method of reducing or maintaining tau (e.g., reducing or maintaining tau, or delaying tau accumulation, tau phosphorylation, and/or tau spreading, or slowing a rate of any of these) in a subject having AD or at risk of developing AD, comprising administering to the subject a therapeutically effective amount of lemborexant, wherein the therapeutically effective amount is sufficient to tau in the subject.

20. The method of claim 19, wherein the subject is amyloid negative.

21. The method of claim 19, wherein the tau levels are reduced or maintained relative to a reference.

22. The method of claim 19, wherein the method comprises reducing and/or delaying tau accumulation and/or tau spreading, and/or slowing a rate thereof, as compared to a reference.

23. The method of claim 22, wherein the reference is a baseline measurement from the subject prior to treatment.

24. The method of claim 22, wherein the reference is a baseline measurement from a control subject.

25. The method of claim 22, wherein the reference is a measurement from a control subject administered a placebo.

26. The method of claim 19, wherein the method comprises altering tau in a brain region of the subject.

27. The method of claim 19, wherein the method comprises altering the tau PET signal in a brain region of the subject.

28. The method of claim 27, wherein the brain region is the hippocampus, entorhinal cortex, and/or the piriform cortex.

29. The method of claim 19, wherein the method comprises reducing tau in a body fluid of the subject.

30. The method of claim 29, wherein the body fluid is blood or CSF.

31. The method of claim 19, wherein the tau is total tau.

32. The method of claim 19, wherein the tau is an insoluble tau.

33. The method of claim 19, wherein the tau is aggregated tau.

34. The method of claim 19, wherein the tau is a phosphorylated form of tau (phospho-tau).

35. The method of claim 34, wherein the phopho-tau is phosphorylated on one or more of T181, T217, S202, S205, or T231.

36. The method of claim 34, wherein the method comprises altering a ratio of phopho-tau to total tau.

37. The method of claim 36, wherein the ratio of phospho-tau to total tau is reduced compared to the ratio of CSF phospho-tau to total tau of the subject prior to administration of lemborexant.

38. The method of claim 36, wherein the ratio of phospho-tau to total tau is maintained within 10% of the ratio of phospho-tau to total tau of the subject prior to the administration of lemborexant.

39. The method of claim 34, comprising increasing a rate of dephosphorylation of phospho-tau.

40. The method of claim 34, comprising reducing a rate of phosphorylation of tau.

41. The method of claim 19, comprising reducing or maintaining tau within 48 hours of administration of a first dose of lemborexant.

42. The method of claim 34, wherein the method comprises reducing phospho-tau in the hippocampus, entorhinal cortex, and/or piriform cortex.

43. A method of altering neurodegeneration (e.g., reducing and/or delaying neurodegeneration, and/or slowing a rate thereof) in a subject having AD or at risk of developing AD, comprising administering to the subject a therapeutically effective amount of lemborexant, a pharmaceutically acceptable salt thereof, or a solvate thereof, wherein the therapeutically effective amount is sufficient to alter neurodegeneration in the subject.

44. The method of claim 43, wherein the subject is amyloid negative.

45. The method of claim 43, wherein altering neurodegeneration comprises reducing and/or delaying neurodegeneration, and/or slowing a rate thereof, as compared to a reference.

46. The method of claim 43, wherein the neurodegeneration is altered relative to a reference.

47. The method of claim 46, wherein the reference is a baseline measurement from the subject prior to treatment.

48. The method of claim 46, wherein the reference is a baseline measurement from a control subject.

49. The method of claim 46, wherein the reference is measurement from a control subject administered a placebo.

50. The method of claim 43, wherein the neurodegeneration is characterized by a loss of at least one of cortical thickness and hippocampal volume.

51. The method of claim 50, wherein altering neurodegeneration comprises maintaining or slowing a loss reduction of cortical thickness and/or hippocampal volume.

52. The method of claim 43, wherein the neurodegeneration is characterized by a loss of at least one of pyramidal neurons in the cortex, pyramidal neurons in the hippocampus, or granule cells in the hippocampus.

53. The method of claim 52, wherein altering neurodegeneration comprises maintaining or reducing loss of pyramidal neurons and/or granule cells.

54. The method of claim 43, wherein altering neurodegeneration comprises reducing a rate of neurodegeneration.

55. The method of claim 43, wherein altering neurodegeneration comprises altering a neurofilament light chain (NfL) level.

56. The method of claim 55, comprising altering the NfL levels in the blood and/or CSF of the subject.

57. A method of altering Aβ plaques (e.g., reducing or delaying formation of Aβ plaques, or slowing a rate of growth thereof) in a subject having AD or at risk of developing AD, comprising administering to the subject a therapeutically effective amount of lemborexant, a pharmaceutically acceptable salt thereof, or a solvate thereof, wherein the therapeutically effective amount is sufficient to alter Aβ plaques in the subject.

58. The method of claim 57, wherein the Aβ plaques are altered relative to a reference.

59. The method of claim 58, wherein altering Aβ plaques comprises reducing and/or delaying formation of Aβ plaques, and/or slowing a rate thereof, as compared to a reference.

60. The method of claim 58, wherein the reference is a baseline measurement from the subject prior to treatment.

61. The method of claim 58, wherein the reference is a baseline measurement from a control subject.

62. The method of claim 58, wherein the reference is a measurement from a control subject administered a placebo.

63. The method of claim 57, wherein the Aβ plaques are fibrillar plaques.

64. The method of claim 57, wherein the Aβ plaques all plaques.

65. The method of claim 57, wherein altering Aβ plaques comprises reducing the growth of Aβ plaques.

66. The method of claim 65, comprising reducing growth of Aβ plaques in the hippocampus of the subject, somatomotor cortex, the somatosensory cortex, and/or the piriform cortex of the subject.

67. The method of claim 57, wherein altering Aβ plaques comprises altering an amyloid PET signal obtained from a brain region of the subject.

68. The method of claim 57, wherein altering Aβ plaques corresponds to a reduction in the concentration of Aβ in the subject's CSF.

69. The method of claim 68, wherein the Aβ is Aβ38, Aβ40, and/or Aβ42.

70. The method of claim 57, comprising altering Aβ plaques within 48 hours of administration of a first dose of lemborexant.

71. The method of claim 57, wherein the subject does not show signs of dementia and/or cognitive impairment.

72. The method of claim 57, wherein the subject has mild cognitive impairment or mild dementia.

73. The method of claim 57, wherein the subject is at risk for further Aβ accumulation.

74. The method of claim 73, wherein the subject is an ApoE4 carrier.

75. The method of claim 73, wherein the subject has intermediate levels of amyloid PET (e.g., 20-40 centiloids).

76. The method of claim 73, wherein the subject has elevated levels of amyloid PET (e.g., >40 centiloids).

77. The method of claim 57, wherein the subject has early-stage AD.

78. The method of claim 57, wherein the subject has pre-AD.

79. A method of modulating a microglial response in a subject having Alzheimer's disease (AD) or at risk of developing AD, comprising administering to the subject a therapeutically effective amount of lemborexant, a pharmaceutically acceptable salt thereof, or a solvate thereof, wherein the therapeutically effective amount is sufficient to modulate the microglial response in the subject.

80. The method of claim 79, wherein modulating the microglial response comprises modulating expression of at least one microglial marker.

81. The method of claim 80, wherein the microglial marker is a general microglial marker.

82. The method of claim 81, wherein the general microglial marker is Iba1, Clec7a, or CD68.

83. The method of claim 80, wherein the microglial marker is a homeostatic microglial marker.

84. The method of claim 83, wherein the homeostatic microglial marker is TMEM119 or P2RY12.

85. The method of claim 79, wherein modulating the microglial response comprises modulating activity of phagocytic microglia.

86. The method of claim 79, wherein the subject has mild cognitive impairment or mild dementia.

87. The method of claim 79, wherein the subject does not show signs of dementia and/or cognitive impairment.

88. The method of claim 79, wherein the subject is amyloid negative.

89. The method of claim 79, wherein the subject has tau pathology.

90. The method of claim 79, wherein the subject has neurodegeneration in a brain region.

91. The method of claim 90, wherein the brain region is the hippocampus, the entorhinal cortex, and/or the piriform cortex.

92. The method of claim 90, wherein the brain region is the CA1 region, the CA2 region, the CA3 region, or the dentate gyrus in the hippocampus.

93. The method of claim 88, wherein modulating the microglial response comprises modulating a response in microglia associated with degenerating neurons.

94. The method of claim 93, wherein modulating the microglial response comprises reducing expression of at least one general microglial marker.

95. The method of claim 94, wherein the general microglial marker is Iba1, CD68, or Clec7a.

96. The method of any claim 93, wherein modulating the microglial response comprises increasing expression of at least one homeostatic microglial marker.

97. The method of claim 96, wherein the homeostatic microglial marker is TMEM119 or P2RY12.

98. The method of claim 79, wherein the subject has Aβ plaques.

99. The method of claim 98, wherein the Aβ plaques are fibrillar Aβ plaques.

100. The method of claim 98, wherein the subject is at risk for further Aβ accumulation.

101. The method of claim 100, wherein the subject is an ApoE4 carrier.

102. The method of claim 100, wherein the subject has intermediate levels of amyloid PET (e.g., 20-40 centiloids).

103. The method of claim 100, wherein the subject has elevated levels of amyloid PET (e.g., >40 centiloids).

104. The method of claim 98, wherein the subject has early-stage AD.

105. The method of claim 98, wherein the subject has pre-AD.

106. The method of claim 98, wherein the Aβ plaques are present in the hippocampus, the somatomotor cortex, the somatosensory cortex, and/or the piriform cortex.

107. The method of claim 98, wherein modulating the microglial response comprises modulating a response in microglia associated with Aβ plaques.

108. The method of claim 107, wherein modulating the microglial response comprises increasing expression of a general microglial marker.

109. The method of claim 108, wherein the general microglial marker is Iba1, Clec7a, or CD68.

110. The method of claim 107, wherein modulating microglial response comprises increasing phagocytosis of Aβ plaques by phagocytic microglia.

111. The method of claim 107, wherein modulating the microglial response comprises reducing expression of a homeostatic microglial marker.

112. The method of claim 111, wherein the homeostatic microglial marker is TMEM119 or P2RY12.

113. A method of any one of claims 1-112, wherein the therapeutically effective amount of lemborexant administered to the subject is in a range of 5 mg to 50 mg per day.

114. The method of claim 113, wherein the therapeutically effective amount of lemborexant administered to the subject is in a range of 10 mg to 30 mg per day.

115. The method of claim 113, wherein the therapeutically effective amount of lemborexant administered to the subject is selected from 5 mg, 7.5 mg, 10 mg, 12.5 mg, 15 mg, 17.5 mg, 20 mg, 22.5 mg, 25 mg, 27.5 mg and 30 mg per day.

116. The method of claim 113, wherein the therapeutically effective amount of lemborexant administered to the subject is 20-25 mg per day.

117. The method of any one of claims 1-112, wherein one dose of 25 mg of lemborexant is administered to the subject once per day.

118. The method of claim 113, wherein lemborexant is administered at a first dose for a first period, a second dose for a second period, and optionally, at a third dose for a third period.

119. The method of claim 118, wherein each of the first period, the second period, and the third period are 1 week.

120. The method of claim 118, wherein the first dose is lower than the second dose, and optionally, the second dose is lower than the third dose.

121. The method of claim 120, wherein the first dose is 5 mg of lemborexant once per day, the second dose is 10 mg of lemborexant once per day, and, optionally, the third dose is 20-25 mg of lemborexant once per day.

122. The method of claim 120, wherein the first dose is 5 mg or 7.5 mg lemborexant once per day, the second dose is 10 mg, 12.5 mg, 15 mg, or 17.5 mg of lemborexant once per day, and the third dose is 20 mg, 22.5 mg, 25 mg, 27.5 mg or 30 mg of lemborexant once per day.

123. The method of claim 118, wherein the first dose is higher than the second dose, and optionally, the second dose is higher than the third dose.

124. The method of claim 123, wherein the first dose is 20-25 mg of lemborexant once per day, the second dose is 10 mg of lemborexant once per day, and, optionally, the third dose is 5 mg of lemborexant once per day.

125. The method of claim 123, wherein the first dose is 20 mg, 22.5 mg, 25 mg, 27.5 mg or 30 mg of lemborexant once per day, the second dose is 10 mg, 12.5 mg, 15 mg, or 17.5 mg of lemborexant once per day, and, optionally, the third dose is 5 mg or 7.5 mg of lemborexant once per day.

126. The method of any one of claims 1-125, comprising administering lemborexant to the subject for at least 6 months.

127. The method of claim 126, comprising lemborexant to the subject for at least 9 months, at least 12 months, or at least 15 months.

128. The method of claim 126, comprising administering lemborexant to the subject for at least 18 months.

129. The method of any one of claims 126-128, comprising administering lemborexant to the subject for at least 24 months, 30 months, or 36 months.

130. A method of selecting a subject having Alzheimer's disease (AD) or at risk of developing AD for treatment with lemborexant, a pharmaceutically acceptable salt thereof, or a solvate thereof, comprising:
(a) obtaining from the subject a measurement of at least one of tau phosphorylation, tau aggregation, neurodegeneration, Aβ plaque burden, microglial response, and biomarker expression;
(b) comparing the measurement from the subject to a measurement from a reference; and
(c) selecting the subject for treatment with lemborexant if the measurement from the subject differs from the measurement from the reference.

131. The method of claim 130, wherein the subject has mild cognitive impairment or mild dementia.

132. The method of claim 130, wherein the subject does not show signs of dementia and/or cognitive impairment.

133. The method of claim 130, wherein the subject is at risk for Aβ accumulation.

134. The method of claim 133, wherein the subject is an ApoE4 carrier.

135. The method of claim 133, wherein the subject has intermediate levels of amyloid PET (e.g., 20-40 centiloids).

136. The method of claim 133, wherein the subject has elevated levels of amyloid PET (e.g., >40 centiloids).

137. The method of claim 130, wherein the subject has early-stage AD.

138. The method of claim 130, wherein the subject has pre-AD.

139. The method of claim 130, wherein the subject has been diagnosed with AD, based on brain imaging, cognitive function, and/or biomarker criteria.

140. The method of claim 130, wherein obtaining at least one measurement comprises obtaining data from a brain scan of the subject and/or obtaining data from a biological sample from the subject.

141. The method of claim 140, wherein the data from the brain scan indicates a level of tau phosphorylation, tau aggregation, Aβ plaque burden, and/or microglial response.

142. The method of claim 140, wherein the biological sample is a body fluid.

143. The method of claim 140, wherein the body fluid is cerebrospinal fluid (CSF), blood, or saliva.

144. The method of claim 130, wherein the reference is a control.

145. The method of claim 130, wherein the reference is a measurement from a control subject administered a placebo.

146. The method of claim 144, wherein the control does not have AD.

147. The method of claim 146, wherein the measurement from the subject is higher than the measurement from the control who does not have AD.

148. The method of claim 146, wherein the measurement from the subject is lower than the measurement from the control who does not have AD.

149. The method of claim 144, wherein the control has AD.

150. The method of claim 149, wherein the measurement from the subject is comparable to or higher than the measurement from the control who has AD.

151. The method of claim 149, wherein the measurement from the subject is comparable to or lower than the measurement from the control who has AD.

152. The method of claim 130, wherein the measurement of tau phosphorylation comprises a measurement of phosphorylation on one more of T181, T217, S202, S205, or T231.

153. The method of claim 130, wherein the measurement of tau aggregation comprises a measurement of insoluble tau aggregates (e.g., neurofibrillary tangles (NFTs)).

154. The method of claim 130, wherein the measurement of neurodegeneration comprises a measurement of cortical thickness and/or hippocampal volume or a measurement of loss of pyramidal neurons or granule neurons.

155. The method of claim 130, wherein the measurement of Aβ plaque burden comprises a measurement of Aβ plaque volume and/or growth of Aβ plaque volume.

156. The method of claim 130, wherein the measurement of Aβ plaque burden comprises a measurement of amyloid PET signal in a brain region of the subject or a measurement of Aβ in the CSF of the subject.

157. The method of claim 130, wherein the measurement of microglial response is a change in the expression of at least one microglial marker.

158. The method of claim 157, wherein the microglial marker is Iba1, Clec7a, CD68, TMEM119, or P2RY12.

159. The method of claim 157, wherein the measurement of microglial response is a measurement of phagocytosis by microglia.

160. A method of monitoring treatment efficacy in a subject having Alzheimer's disease (AD) or at risk of developing AD, comprising:
(a) obtaining from the subject a first measurement of at least one of tau phosphorylation, tau aggregation, neurodegeneration, Aβ plaque burden, microglia function, and biomarker expression;
(b) administering to the subject a dose of lemborexant, a pharmaceutically acceptable salt thereof, or a solvate thereof;
(c) obtaining from the subject a second measurement of at least one of tau phosphorylation, tau aggregation, neurodegeneration, Aβ plaque burden, microglia function, and biomarker expression; and
(d) comparing the second measurement from the subject to the first measurement from the subject,
wherein a difference between the first measurement and the second measurement indicates effective treatment with lemborexant.

161. A method of treating a subject having Alzheimer's disease (AD) or at risk of developing AD, comprising:
(a) obtaining from the subject a first measurement of at least one of tau phosphorylation, tau aggregation, neurodegeneration, Aβ plaque burden, microglial response, and biomarker expression;
(b) administering to the subject a first dose of lemborexant, a pharmaceutically acceptable salt thereof, or a solvate thereof;
(c) obtaining from the subject a second measurement of at least one of tau phosphorylation, tau aggregation, neurodegeneration, Aβ plaque burden, microglia function, and biomarker expression;
(d) comparing the second measurement from the subject to the first measurement from the subject, and
(e) administering a second dose of lemborexant if the first measurement differs from the second measurement.

162. The method of claim 160 or claim 161, wherein obtaining at least one measurement comprises obtaining data from a brain scan of the subject and/or obtaining data from a biological sample from the subject.

163. The method of claim 162, wherein the data from the brain scan indicates a level of tau phosphorylation, tau aggregation, Aβ plaque burden, and/or microglial response.

164. The method of claim 162, wherein the biological sample is a body fluid.

165. The method of claim 164, wherein the body fluid is cerebrospinal fluid (CSF), blood, or saliva.

166. The method of claim 160 or claim 161, wherein the first measurement from the subject is higher than the second measurement from the subject.

167. The method of claim 160 or claim 161, wherein the first measurement from the subject is lower than the second measurement from the subject.

168. The method of claim 160 or claim 161, wherein the measurement of tau phosphorylation comprises a measurement of phosphorylation of one or more of T181, T217, S202, S205, or T231.

169. The method of claim 160 or claim 161, wherein the measurement of tau aggregation comprises a measurement of insoluble tau aggregates (e.g., neurofibrillary tangles (NFTs)).

170. The method of claim 160 or claim 161, wherein the measurement of neurodegeneration comprises a measurement of cortical thickness and/or hippocampal volume or a measurement of loss of pyramidal neurons or granule neurons.

171. The method of claim 160 or claim 161, wherein the measurement of Aβ plaque burden comprises a measurement of Aβ plaque volume and/or growth of Aβ plaque volume.

172. The method of claim 160 or claim 161, wherein the measurement of Aβ plaque burden comprises a measurement of amyloid PET signal in a brain region of the subject or a measurement of Aβ in the CSF of the subject.

173. The method of claim 160 or claim 161, wherein the measurement of microglial response is a measure of expression of at least one microglial marker.

174. The method of claim 173, wherein the microglial marker is Iba1, Clec71, P2RY12 or TMEM 119.

175. The method of claim 173, wherein the measurement of microglial response is a measurement of phagocytosis by microglia.

176. The method of any claim 160 or claim 161, wherein the measurement of a biomarker expression is a measurement of Ifnb1, MMP2, and/or Bace1 expression.

177. The method of claim 160 or claim 161, wherein the subject is amyloid-negative.

178. The method of claim 160 or claim 160, wherein the subject has Aβ plaques.

179. The method of claim 160 or claim 161, wherein the subject has mild cognitive impairment and/or mild dementia.

180. The method of claim 160 or claim 161, wherein the subject does not show signs of dementia and/or cognitive impairment.

181. The method of claim 160 or claim 161, wherein the subject is at risk for further Aβ accumulation.

182. The method of claim 181, wherein the subject is an ApoE4 carrier.

183. The method of claim 181, wherein the subject has intermediate levels of amyloid PET (e.g., 20-40 centiloids).

184. The method of claim 181, wherein the subject has elevated levels of amyloid PET (e.g., >40 centiloids).

185. The method of claim 160 or claim 161, wherein the subject has early-AD.

186. The method of claim 160 or claim 161, wherein the subject has pre-AD.
